(19) United States
(12) Patent Application Publication
(10) Pub. No.: US 2026/0146294 A1
(43) Pub. Date: May 28, 2026

(54) METHODS TO ENRICH NUCLEOTIDE VARIANTS BY NEGATIVE SELECTION

(71) Applicant: GUARDANT HEALTH, INC., Palo Alto, CA (US)

(72) Inventor: Andrew KENNEDY, San Diego, CA (US)

(21) Appl. No.: 19/461,369

(22) Filed: Jan. 27, 2026

Related U.S. Application Data
(63) Continuation of application No. PCT/US2024/038192, filed on Jul. 16, 2024.
(60) Provisional application No. 63/516,422, filed on Jul. 28, 2023.
(60) Provisional application No. 63/561,589, filed on Mar. 5, 2024.

(51) Int. Cl. C12Q 1/6886; C12Q 1/6806

(52) U.S. Cl. CPC C12Q 1/6886; C12Q 1/6806; C12Q 2600/154; C12Q 2600/156

(57) ABSTRACT

Provided herein are methods for selectively depleting a target nucleic acid comprising a sequence that does not comprise a nucleotide variant of interest. In some embodiments, a concentration of the target nucleic acid comprising the variant sequence is increased relative to a concentration of the target nucleic acid comprising the wild-type sequence, a concentration of the target nucleic acid comprising a converted nucleotide is increased relative to a concentration of the target nucleic acid that does not comprise the converted nucleotide, or a concentration of the target nucleic acid that does not comprise the converted nucleotide is increased relative to a concentration of the target nucleic acid comprising the converted nucleotide. In some embodiments, the target nucleic acids are sequenced.

### CROSS-REFERENCE TO RELATED APPLICATIONS

The present application is a Continuation of International Patent Application No. PCT/US2024/038192, filed Jul. 16, 2024, which claims the benefit of priority of U.S. Provisional Patent Application No. 63/516,422, filed Jul. 28, 2023, and U.S. Provisional Patent Application No. 63/561,589, filed Mar. 5, 2024, each of which is incorporated by reference herein in its entirety for all purposes.

### SEQUENCE LISTING

The present application contains a sequence listing that has been submitted electronically in XML format. Said XML copy, created on Jul. 9, 2024, is named “01228-0035-00PCT-ST26.xml” and is 19,441 bytes in size. The information in the electronic format of the sequence listing is incorporated herein by reference in its entirety.

### FIELD OF THE INVENTION

The present disclosure provides methods related to selectively depleting a target nucleic acid comprising a sequence that does not comprise a nucleotide variant of interest. In some embodiments, the method selectively depletes a target nucleic acid comprising a wild-type sequence, a target nucleic acid comprising a converted nucleotide, or a target nucleic acid that does not comprise a converted nucleotide. In some embodiments, the target nucleic acid is from a subject having or suspected of having a disease or disorder, such as cancer.

### INTRODUCTION AND SUMMARY

Cancer is responsible for millions of deaths per year worldwide. Early detection of cancer may result in improved outcomes because early-stage cancer tends to be more susceptible to treatment.

Improperly controlled cell growth is a hallmark of cancer that generally results from an accumulation of genetic and epigenetic changes, such as copy number variations (CNVs), single nucleotide variations (SNVs), gene fusions, insertions and/or deletions (indels), epigenetic variations including modification of cytosine (e.g., 5-methylcytosine, 5-hydroxymethylcytosine, and other more oxidized forms) and association of DNA with chromatin proteins and transcription factors. Thus, cancer can be indicated by sequence modifications and/or non-sequence modifications, such as methylation. Examples of methylation changes in cancer include local gains of DNA methylation in the CpG islands at the TSS of genes involved in normal growth control, DNA repair, cell cycle regulation, and/or cell differentiation. Hypermethylation can be associated with an aberrant loss of transcriptional capacity of involved genes and occurs at least as frequently as point mutations and deletions as a cause of altered gene expression. Furthermore, without wishing to be bound by any particular theory, cells in or around a cancer or neoplasm may shed more DNA than cells of the same tissue type in a healthy subject. The DNA from such cells may differ epigenetically from shed DNA in a healthy subject. As such, the distribution of epigenetically modified (e.g., methylated) DNA in certain DNA samples, such as cell-free DNA (cfDNA), may change upon carcinogenesis. Thus, sufficiently sensitive epigenetic (e.g., DNA methylation) profiling can be used to detect aberrant methylation in DNA of a sample.

Current methods of cancer diagnostic assays of nucleic acids (e.g., cell-free DNA or cell-free RNA) may focus on the detection of tumor-related somatic variants, including SNVs, copy CNVs, fusions, and indels. There is also growing evidence that non-sequence modifications like methylation status and fragmentomic signal can provide information on the source of nucleic acids and disease level. In addition, different types of modifications such as 5-methylation and 5-hydroxymethylation can have different implications as to the presence or absence of disease. Detailed knowledge of the sequence and non-sequence modifications of nucleic acids can improve assessments of tumor status. Targeted next-generation sequencing assay workflows that use molecular barcodes enable high performance detection of many nucleotide variants. However, nucleotide variants (including both sequence and non-sequence related variants) may be very rare compared the wild-type DNA, making it costly to sequence at enough depth to achieve useful sensitivity in detecting variants. This may particularly be an issue in liquid biopsy samples (such as blood and/or cfDNA samples), in which variants may constitute 0.01% or less of total nucleic acids. Attempts have been made to enrich nucleotide variant molecules in diagnostic workflows, but most such attempts have been limited in target-plex and are specific to a particular individual variant sequence at a given locus. The latter is problematic as more than one type of nucleotide variant at a particular locus may be relevant, e.g., to detecting or diagnosing a disease (such as a cancer) in a subject; for example, in the case of the proto-oncogene KRAS, multiple different mutations of glycine 12 are associated with oncogenesis.

Accordingly, there is a continued need for improved sensitivity in methods and compositions for analyzing variants present in nucleic acids, such as in RNA and cell-free DNA, e.g., in nucleic acids from liquid biopsies.

The present disclosure aims to meet the need for improved sensitivity in nucleic acid analysis, such as in analysis of RNA and cell-free DNA, and/or provide other benefits. In some embodiments, the present disclosure provides a method of selectively depleting a target nucleic acid comprising a wild-type sequence, a target nucleic acid comprising a converted nucleotide, or a target nucleic acid that does not comprise a converted nucleotide. Such methods allow for selective depletion of wild-type sequences (including sequences comprising wild-type epigenetic modifications) in target nucleic acids, such as in cell-free DNA or cell-free RNA. The disclosed methods thereby increase the relative proportions of nucleic acids comprising one or more variants (i.e., enrich for nucleic acids comprising the one or more variants) at a given locus as compared to nucleic acids comprising a wild-type (or other non-desired) sequence at the same locus. The enriching occurs independently of the identity of the nucleotide variant at the given locus, and the disclosed methods may reduce a sequencing depth required to detect and analyze the one or more variants.

The following exemplary embodiments are provided.

Embodiment 1 is a method of selectively depleting a target nucleic acid comprising a wild-type sequence, the method comprising:
(a) contacting a population of target nucleic acids with an oligonucleotide probe, wherein:
   (i) the population of target nucleic acids comprises a target nucleic acid comprising a wild-type sequence and comprises or is suspected of comprising a target nucleic acid comprising a variant sequence; and
   (ii) the oligonucleotide probe preferentially forms a substrate for extension with the wild-type sequence relative to the variant sequence;
(b) extending the oligonucleotide probe, wherein the extending produces a partially double-stranded target nucleic acid comprising the wild-type sequence; and
(c) selectively digesting the partially double-stranded target nucleic acid comprising the wild-type sequence;
thereby selectively depleting the target nucleic acid comprising the wild-type sequence.

Embodiment 2 is a method of selectively depleting a target nucleic acid that comprises a converted nucleotide, the method comprising:
(a) subjecting a population of target nucleic acids from a subject to a conversion procedure that affects a first nucleobase of the target nucleic acids differently from a second nucleobase of the target nucleic acids, wherein the first nucleobase is a modified or unmodified nucleobase, the second nucleobase is a modified or unmodified nucleobase different from the first nucleobase, and the first nucleobase and the second nucleobase have the same base pairing specificity, thereby providing a population of converted target nucleic acids;
(b) contacting the population of converted target nucleic acids with an oligonucleotide probe, wherein the oligonucleotide probe preferentially forms a substrate for extension with a target nucleic acid that comprises a converted nucleotide relative to a target nucleic acid that does not comprise the converted nucleotide;
(c) extending the oligonucleotide probe, wherein the extending produces a partially double-stranded target nucleic acid that comprises the converted nucleotide; and
(d) selectively digesting the partially double-stranded target nucleic acid that comprises the converted nucleotide;
thereby selectively depleting the target nucleic acid that comprises the converted nucleotide.

Embodiment 3 is a method of selectively depleting a target nucleic acid that does not comprise a converted nucleotide, the method comprising:
(a) subjecting a population of target nucleic acids to a conversion procedure that affects a first nucleobase of the target nucleic acids differently from a second nucleobase of the target nucleic acids, wherein the first nucleobase is a modified or unmodified nucleobase, the second nucleobase is a modified or unmodified nucleobase different from the first nucleobase, and the first nucleobase and the second nucleobase have the same base pairing specificity, thereby providing a population of converted target nucleic acids;
(b) contacting the population of converted target nucleic acids with an oligonucleotide probe, wherein the oligonucleotide probe preferentially forms a substrate for extension with a target nucleic acid that does not comprise a converted nucleotide relative to a target nucleic acid that comprises the converted nucleotide;
(c) extending the oligonucleotide probe, wherein the extending produces a partially double-stranded target nucleic acid that does not comprise the converted nucleotide; and
(d) selectively digesting the partially double-stranded target nucleic acid that does not comprise the converted nucleotide;
thereby selectively depleting the target nucleic acid that does not comprise the converted nucleotide.

Embodiment 4 is the method of any one of embodiments 1 to 3, further comprising sequencing the population of target nucleic acids after selectively digesting the partially double-stranded target nucleic acid comprising the wild-type sequence, the partially double-stranded target nucleic acid that comprises the converted nucleotide, or the partially double-stranded target nucleic acid that does not comprise the converted nucleotide.

Embodiment 5 is a method of selectively depleting a target nucleic acid comprising a wild-type sequence, the method comprising:
(a) contacting a population of target nucleic acids with an oligonucleotide probe, wherein:
   (i) substantially each target nucleic acid of the population of target nucleic acids is single-stranded and comprises an adapter comprising a restriction enzyme cleavage site and a universal primer binding site;
   (ii) the population of target nucleic acids comprises a target nucleic acid comprising a wild-type sequence and comprises or is suspected of comprising a target nucleic acid comprising a variant sequence; and
   (iii) the oligonucleotide probe preferentially forms a substrate for extension with the wild-type sequence relative to the variant sequence;
(b) extending the oligonucleotide probe, wherein the extending produces a partially double-stranded target nucleic acid comprising the wild-type sequence;
(c) selectively digesting the partially double-stranded target nucleic acid comprising the wild-type sequence, thereby producing a selectively digested population of target nucleic acids;
(d) amplifying the selectively digested population of target nucleic acids exponentially, thereby producing a population of amplified target nucleic acids; and
(e) sequencing the population of amplified target nucleic acids.

Embodiment 6 is a method of selectively depleting a target nucleic acid that comprises a converted nucleotide, the method comprising:
(a) subjecting a population of target nucleic acids to a conversion procedure that affects a first nucleobase of the target nucleic acids differently from a second nucleobase of the target nucleic acids, wherein
   (i) substantially each target nucleic acid of the population of target nucleic acids is single-stranded and comprises an adapter comprising a restriction enzyme cleavage site and a universal primer binding site; and
   (ii) the first nucleobase is a modified or unmodified nucleobase, the second nucleobase is a modified or unmodified nucleobase different from the first nucleobase, and the first nucleobase and the second nucleobase have the same base pairing specificity;
thereby providing a population of converted target nucleic acids;
(b) contacting the population of converted target nucleic acids with an oligonucleotide probe, wherein the oligonucleotide probe preferentially forms a substrate for extension with a target nucleic acid that comprises a converted nucleotide relative to a target nucleic acid that does not comprise the converted nucleotide;
(c) extending the oligonucleotide probe, wherein the extending produces a partially double-stranded target nucleic acid that comprises the converted nucleotide; and
(d) selectively digesting the partially double-stranded target nucleic acid that comprises the converted nucleotide, thereby producing a selectively digested population of target nucleic acids;
(e) amplifying the selectively digested population of target nucleic acids exponentially, thereby producing a population of amplified target nucleic acids; and
(f) sequencing the population of amplified target nucleic acids.

Embodiment 7 is a method of selectively depleting a target nucleic acid that does not comprise a converted nucleotide, the method comprising:
(a) subjecting a population of target nucleic acids to a conversion procedure that affects a first nucleobase of the target nucleic acids differently from a second nucleobase of the target nucleic acids, wherein
   (i) substantially each target nucleic acid of the population of target nucleic acids is single-stranded and comprises an adapter comprising a restriction enzyme cleavage site and a universal primer binding site; and
   (ii) the first nucleobase is a modified or unmodified nucleobase, the second nucleobase is a modified or unmodified nucleobase different from the first nucleobase, and the first nucleobase and the second nucleobase have the same base pairing specificity;
thereby providing a population of converted target nucleic acids;
(b) contacting the population of converted target nucleic acids with an oligonucleotide probe, wherein the oligonucleotide probe preferentially forms a substrate for extension with a target nucleic acid that does not comprise a converted nucleotide relative to a target nucleic acid that comprises the converted nucleotide;
(c) extending the oligonucleotide probe, wherein the extending produces a partially double-stranded target nucleic acid that does not comprise the converted nucleotide; and
(d) selectively digesting the partially double-stranded target nucleic acid that does not comprise the converted nucleotide, thereby producing a selectively digested population of target nucleic acids;
(e) amplifying the selectively digested population of target nucleic acids exponentially, thereby producing a population of amplified target nucleic acids; and
(f) sequencing the population of amplified target nucleic acids.

Embodiment 8 is the method of any one of the embodiments 1, 4, or 5, wherein the population of target nucleic acids comprises the target nucleic acid comprising the variant sequence.

Embodiment 9 is the method of any one of the embodiments 1 to 8, wherein
(a) the extending does not produce a partially double-stranded target nucleic acid comprising the variant sequence;
(b) the method selectively depletes a target nucleic acid that comprises the converted nucleotide and the extending does not produce a partially double-stranded target nucleic acid that does not comprise the converted nucleotide; or
(c) the method selectively depletes the target nucleic acid that does not comprise the converted nucleotide and the extending does not produce a partially double-stranded target nucleic acid that comprises the converted nucleotide.

Embodiment 10 is the method of any one of the embodiments 1 to 9, wherein a first portion of the oligonucleotide probe
(a) is complementary to a portion of the target nucleic acid comprising the wild-type sequence, and is not complementary to a portion of the target nucleic acid comprising the variant sequence;
(b) is complementary to a portion of the target nucleic acid that does not comprise the converted nucleotide, and is not complementary to a portion of the target nucleic acid that comprises the converted nucleotide; or
(c) is complementary to a portion of the target nucleic acid that comprises the converted nucleotide, and is not complementary to a portion of the target nucleic acid that does not comprise the converted nucleotide.

Embodiment 11 is the method of any one of the embodiments 1 to 10, wherein a second portion of the oligonucleotide probe
(a) is complementary to both a portion of the target nucleic acid comprising the wild-type sequence and a portion of the target nucleic acid comprising the variant sequence; or
(b) is complementary to both a portion of the target nucleic acid that does not comprise the converted nucleotide and a portion of the target nucleic acid that comprises the converted nucleotide.

Embodiment 12 is the method of embodiment 10 or 11, wherein the first portion of the oligonucleotide probe comprises a 3′ end of the oligonucleotide probe.

Embodiment 13 is the method of any one of embodiments 10 to 12, wherein the second portion of the oligonucleotide probe comprises a 5′ end of the oligonucleotide probe.

Embodiment 14 is the method of any one of embodiments 2, 3, 6, 7, or 6 to 13, wherein the method selectively depletes a target nucleic acid that comprises the converted nucleotide and the converted nucleotide is bound by the 3′ nucleotide of the oligonucleotide probe, or the method selectively depletes the target nucleic acid that does not comprise the converted nucleotide and the converted nucleotide is not bound by the 3′ nucleotide of the oligonucleotide probe.

Embodiment 15 is the method of any one of the embodiments 1 to 14, further comprising attaching one or more adapters to the target nucleic acid, optionally wherein the attaching comprises ligating.

Embodiment 16 is the method of any one of the embodiments 1 to 15, wherein the target nucleic acid comprises a 5′ adapter, a 3′ adapter, or both a 5′ adapter and a 3′ adapter.

Embodiment 17 is the method of the embodiment 16, wherein the 5′ adapter, or both the 5′ adapter and the 3′ adapter comprise at least one sequence that is recognized by at least one restriction enzyme.

Embodiment 18 is the method of embodiment 16 or 17, wherein the 5′ adapter is downstream of an oligonucleotide probe binding site within the target nucleic acid.

Embodiment 19 is the method of any one of the embodiments 16 to 18, wherein the 5′ adapter, or both the 5′ adapter and the 3′ adapter, comprises a universal primer binding site.

Embodiment 20 is the method of any one of embodiments 16 to 19, wherein the 5′ adapter, or both the 5′ adapter and the 3′ adapter, comprises a barcode.

Embodiment 21 is the method of any one of the embodiments 1 to 20, further comprising contacting the population of nucleic acids with a second oligonucleotide probe complementary to the strand of the target nucleic acid opposite the strand to which the first oligonucleotide probe is complementary.

Embodiment 22 is the method of any one of the embodiments 1 to 21, wherein extending the oligonucleotide probe comprises contacting the substrate for extension with a DNA polymerase that lacks 3′->5′ exonuclease activity.

Embodiment 23 is the method of any one of the embodiments 1 to 22, wherein selectively digesting the partially double-stranded target nucleic acid comprising the wild-type sequence, the partially double-stranded target nucleic acid that comprises the converted nucleotide, or the partially double-stranded target nucleic acid that does not comprise the converted nucleotide results in substantially linear amplification of the selectively digested target nucleic acid.

Embodiment 24 is the method of any one of the embodiments 1 to 23, wherein a concentration of the target nucleic acid comprising the variant sequence is increased relative to a concentration of the target nucleic acid comprising the wild-type sequence, a concentration of the target nucleic acid comprising the converted nucleotide is increased relative to a concentration of the target nucleic acid that does not comprise the converted nucleotide, or a concentration of the target nucleic acid that does not comprise the converted nucleotide is increased relative to a concentration of the target nucleic acid comprising the converted nucleotide.

Embodiment 25 is the method of embodiment 24, wherein
(a) a post-selective digestion ratio of the target nucleic acid comprising the variant sequence to the target nucleic acid comprising the wild-type sequence is at least 1500:1, 1000:1, 500:1, 100:1, 10:1, 5:1, 1:1, 2:1, 3:1, 4:1, 5:1, 10:1, 100:1, or 1000:1;
(b) a post-selective digestion ratio of the target nucleic acid comprising the unconverted nucleotide to the target nucleic acid comprising converted nucleotide is at least 1500:1, 1000:1, 500:1, 100:1, 10:1, 5:1, 1:1, 2:1, 3:1, 4:1, 5:1, 10:1, 100:1, or 1000:1; or
(c) a post-selective digestion ratio of the target nucleic acid comprising the converted nucleotide to the target nucleic acid comprising the unconverted nucleotide is at least 1500:1, 1000:1, 500:1, 100:1, 10:1, 5:1, 1:1, 2:1, 3:1, 4:1, 5:1, 10:1, 100:1, or 1000:1.

Embodiment 26 is the method of any one of the embodiments 1 to 25, wherein selectively digesting the partially double-stranded target nucleic acid comprising the wild-type sequence, selectively digesting the partially double-stranded target nucleic acid that comprises the converted nucleotide, or selectively digesting the partially double-stranded target nucleic acid that does not comprise the converted nucleotide comprises contacting the population of target nucleic acids with at least one restriction endonuclease.

Embodiment 27 is the method of embodiment 26, wherein the at least one restriction endonuclease cleaves a double-stranded nucleic acid and/or a partially double stranded nucleic acid, and does not cleave a single-stranded nucleic acid.

Embodiment 28 is the method of any one of embodiments 17-27, wherein the at least one restriction endonuclease cleaves the partially double-stranded target nucleic acid at the at least one sequence within the at least one adapter.

Embodiment 29 is the method of any one of embodiments 17-28, wherein the at least one restriction endonuclease is BspQI, AflIII, BsiHKAI, BtrI, MaeII, SduI, HpaII, BstUI, Hin6I, SsiI, or HpyCH4IV.

Embodiment 30 is the method of any one of embodiments 17-29, wherein the at least one restriction endonuclease cleaves a double-stranded nucleic acid at a 5′-GCTCTTCN-3′ cleavage site in an adapter.

Embodiment 31 is the method of any one of embodiments 17-30, wherein the at least one restriction endonuclease is BspQI.

Embodiment 32 is the method of any one of embodiments 17-31, wherein the oligonucleotide probe, or the first and the second oligonucleotide probes, do not comprise the cleavage site.

Embodiment 33 is the method of any one of the embodiments 1 to 32, wherein the population of target nucleic acids comprises DNA or cDNA.

Embodiment 34 is the method of any one of embodiments 1, 4, 5, 8-13, or 15-33, wherein the population of target nucleic acids comprises RNA and the method further comprises a cDNA synthesis step.

Embodiment 35 is the method of any one of embodiments 1, 4, 5, 8-13, or 15-34, wherein the variant sequence is a single nucleotide variant (SNV), an insertion or deletion (indel), a translocation, a gene fusion, or an epigenetic modification.

Embodiment 36 is the method of any one of embodiments 1, 4, 5, 8-13, or 15-35, wherein the variant sequence is an SNV, and wherein the oligonucleotide probe comprises a 3′ portion that is complementary to the target nucleic acid comprising the wild-type sequence and is not complementary to the target nucleic acid comprising the SNV.

Embodiment 37 is the method of any one of embodiments 1, 4, 5, 8-13, or 15-35, wherein the variant sequence is an RNA fusion, and wherein the oligonucleotide probe comprises a 3′ portion that is complementary to the target nucleic acid comprising the wild-type sequence and is not complementary to the target nucleic acid comprising the gene fusion.

Embodiment 38 is the method of any one of embodiments 1, 4, 5, 8-13, or 15-35, wherein the variant sequence is a translocation, and wherein the oligonucleotide probe comprises a 3′ portion that is complementary to the target nucleic acid comprising the wild-type sequence and is not complementary to the target nucleic acid comprising the translocation.

Embodiment 39 is the method of any one of embodiments 2-4, 6, 7, or 14-33, wherein the variant sequence is an epigenetic modification, and wherein the oligonucleotide probe comprises a 3′ portion that is complementary to the target nucleic acid comprising the converted nucleotide and is not complementary to the target nucleic acid that does not comprise the converted nucleotide.

Embodiment 40 is the method of any one of embodiments 2-4, 6, 7, or 14-33, wherein the variant sequence is an epigenetic modification, and wherein the oligonucleotide probe comprises a 3′ portion that is complementary to the target nucleic acid that does not comprise the converted nucleotide and is not complementary to the target nucleic acid comprising the converted nucleotide.

Embodiment 41 is the method of any one of embodiments 36-40, wherein the 3′ portion of the oligonucleotide probe comprises one, two, three, four, or more than four of the 3′-most nucleotides of the oligonucleotide probe.

Embodiment 42 is the method of any one of the embodiments 1 to 41, further comprising partitioning the population of target nucleic acids into a plurality of subsamples, the plurality comprising a first subsample and a second subsample, wherein the partitioning is performed prior to the sequencing and/or
(a) prior to the selectively depleting the target nucleic acid comprising the wild-type sequence, the target nucleic acid comprising the converted nucleotide, or the target nucleic acid that does not comprise the converted nucleotide;
(b) prior to the amplifying the selectively digested population of target nucleic acids;
(c) prior to the subjecting the population of target nucleic acids to a procedure that affects a first nucleobase of the DNA differently from a second nucleobase of the DNA; and/or
(d) prior to a step of enriching for one or more sets of target regions of DNA.

Embodiment 43 is the method of embodiment 42, wherein the partitioning comprises partitioning on the basis of methylation level.

Embodiment 44 is the method of embodiment 42 or embodiment 43, wherein the partitioning comprises contacting the population of target nucleic acids with an agent that recognizes a modified nucleobase in the DNA, wherein the first subsample comprises DNA with the modified nucleobase in a greater proportion than the second subsample.

Embodiment 45 is the method of embodiment 44, wherein the agent that recognizes a modified nucleobase in the DNA is a methyl binding reagent.

Embodiment 46 is the method of embodiment 45, wherein the methyl binding reagent is a methyl binding domain (MBD) protein or an antibody.

Embodiment 47 is the method of embodiment 44 or embodiment 45, wherein the methyl binding reagent is specific to one or more methylated nucleotide bases, optionally wherein the one or more methylated nucleotide bases is 5-methylcytosine.

Embodiment 48 is the method of any one of embodiments 45-47, wherein the methyl binding reagent is immobilized on a solid support.

Embodiment 49 is the method of any one of embodiments 42-48, wherein the partitioning comprises immunoprecipitation of methylated DNA.

Embodiment 50 is the method of any one of embodiments 42-49, wherein the partitioning comprises partitioning on the basis of binding to a protein, optionally wherein the protein is a methylated protein, an acetylated protein, an unmethylated protein, an unacetylated protein; and/or optionally wherein the protein is a histone.

Embodiment 51 is the method of embodiment 50, wherein the partitioning comprises contacting the DNA with a binding reagent which is specific for the protein and is immobilized on a solid support.

Embodiment 52 method of any one of embodiments 42-51, wherein a first partitioned subsample of the plurality of partitioned subsamples is differentially tagged from a second partitioned subsample of the plurality of partitioned subsamples.

Embodiment 53 is the method of any one of embodiments 2-4, 6, 7, 14-33, or 39-52, wherein the first nucleobase is an unmodified cytosine and the second nucleobase is a modified cytosine, optionally wherein the modified cytosine is 5-methylcytosine or 5-hydroxymethylcytosine.

Embodiment 54 is the method of any one of embodiments 1, 4, 5, 8-13, 15-38, or 41-53, wherein the method further comprises subjecting the DNA or a subsample thereof to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity, thereby providing a population of converted target nucleic acids.

Embodiment 55 is the method of any one of embodiments 2-4, 6, 7, 14-33, or 39-54, wherein the procedure that affects a first nucleobase of the DNA differently from a second nucleobase of the DNA is performed prior to the sequencing and/or
(a) prior to the selectively depleting the target nucleic acid comprising the wild-type sequence, the target nucleic acid comprising the converted nucleotide, or the target nucleic acid that does not comprise the converted nucleotide;
(b) prior to the amplifying the selectively digested population of target nucleic acids;
(c) prior to the partitioning the population of target nucleic acids into a plurality of subsamples; and/or
(d) prior to a step of enriching for one or more sets of target regions of DNA.

Embodiment 56 is the method of any one of embodiments 2-4, 6, 7, 14-33, or 39-55, wherein the first nucleobase is an unmodified cytosine and the second nucleobase is a modified cytosine, optionally wherein the modified cytosine is 5-methylcytosine or 5-hydroxymethylcytosine.

Embodiment 57 is the method of any one of embodiments 2-4, 6, 7, 14-33, or 39-56, wherein the procedure that affects a first nucleobase of the DNA differently from a second nucleobase of the DNA chemically converts the first or second nucleobase such that the base pairing specificity of the converted nucleobase is altered.

Embodiment 58 is the method of any one of embodiments 2-4, 6, 7, 14-33, or 39-57, wherein the procedure that affects a first nucleobase of the DNA differently from a second nucleobase of the DNA is methylation-sensitive conversion.

Embodiment 59 is the method of embodiment 58, wherein the methylation-sensitive conversion is bisulfite conversion, oxidative bisulfite (Ox-BS) conversion, Tet-assisted bisulfite (TAB) conversion, APOBEC-coupled epigenetic (ACE) conversion, or enzymatic conversion.

Embodiment 60 is the method of embodiment 59, wherein the Tet-assisted conversion further comprises a substituted borane reducing agent, optionally wherein the substituted borane reducing agent is 2-picoline borane, borane pyridine, tert-butylamine borane, or ammonia borane.

Embodiment 61 is the method of any one of embodiments 1-60, further comprising enriching for one or more sets of target regions of DNA, RNA, or cDNA prepared from the RNA, wherein the one or more sets of target regions comprises one or more of a sequence-variable target region set and an epigenetic target region set, thereby providing enriched DNA, RNA, or cDNA.

Embodiment 62 is the method of embodiment 61, wherein the enriching is performed prior to the sequencing and/or
(a) prior to the selectively depleting the target nucleic acid comprising the wild-type sequence, the target nucleic acid comprising the converted nucleotide, or the target nucleic acid that does not comprise the converted nucleotide;
(b) prior to the amplifying the selectively digested population of target nucleic acids;
(c) prior to the partitioning the population of target nucleic acids into a plurality of subsamples; and/or
(d) prior to the subjecting the population of target nucleic acids to the conversion procedure that affects a first nucleobase of the DNA differently from a second nucleobase of the DNA.

Embodiment 63 is the method of embodiment 61 or embodiment 62, wherein the enriching comprises contacting the DNA, RNA, or cDNA prepared from the RNA, with target-specific probes specific for the one or more sets of epigenetic target regions and/or for the one or more sets of sequence-variable target regions.

Embodiment 64 is the method of any one of embodiments 61-63 wherein the epigenetic target region set comprises a hypermethylation variable target region set and/or a hypomethylation variable target region set.

Embodiment 65 is the method of any one of embodiments 61-64, wherein the epigenetic target region set comprises a fragmentation variable target region set.

Embodiment 66 is the method of embodiment 65, wherein the fragmentation variable target region set comprises transcription start site regions.

Embodiment 67 is the method of embodiment 65 or 66, wherein the fragmentation variable target region set comprises CTCF binding regions.

Embodiment 68 is the method of any one of embodiments 61-67, wherein the epigenetic target region set comprises one or more type-specific epigenetic target regions.

Embodiment 69 is the method of embodiment 68, wherein the one or more type-specific epigenetic target regions comprises type-specific differentially methylated regions and/or type specific fragments.

Embodiment 70 is the method of embodiment 68, wherein the one or more type-specific epigenetic target regions comprises type-specific hypomethylated regions and/or type-specific hypermethylated regions.

Embodiment 71 is the method of any one of embodiments 68-70, wherein the one or more type-specific epigenetic target regions comprises cell-type specific, cell cluster-type specific, tissue-type specific, and/or cancer-type specific epigenetic target regions.

Embodiment 72 is the method of any one of embodiments 68-71, wherein the one or more type-specific epigenetic target regions comprise target regions that are:
hypermethylated in immune cells relative to non-immune cell types present in a blood sample;
differentially methylated in colon relative to other tissue types;
differentially methylated in breast relative to other tissue types;
differentially methylated in liver relative to other tissue types;
differentially methylated in kidney relative to other tissue types;
differentially methylated in pancreas relative to other tissue types;
differentially methylated in prostate relative to other tissue types;
differentially methylated in skin relative to other tissue types; or
differentially methylated in bladder relative to other tissue types.

Embodiment 73 is the method of any one of embodiments 70-72, wherein the hypermethylated target regions are methylated to an extent that is at least 10%, 20%, 30%, or at least 40% greater than the average methylation of the target regions in the sample or relative to other cell or tissue types.

Embodiment 74 is the method of any one of embodiments 68-73, wherein the one or more type-specific epigenetic target regions comprises
target regions that are hypomethylated in non-immune cell types present in the sample relative to the methylation level of the target regions in a different cell or tissue type in the sample;
fragments specific to immune cells relative to non-immune cell types present in the sample; or
fragments specific to colon, lung, breast, liver, kidney, pancreas, prostate, skin, or bladder relative to other tissue types.

Embodiment 75 is the method of any one of embodiments 68-74, wherein the level of the one or more type-specific epigenetic target regions that originated from a cell type or a tissue type is determined.

Embodiment 76 is the method of any one of embodiments 68-75, wherein the levels of the one or more type-specific epigenetic target regions that originated from one or more immune cells, non-immune cell types present in a blood sample, and/or colon, lung, breast, liver, kidney, prostate, skin, bladder, or pancreas cells are determined.

Embodiment 77 is the method of any one of embodiments 68-76, further comprising identifying at least one cell type, cell cluster type, tissue type, and/or cancer type from which the one or more type-specific epigenetic target regions originated.

Embodiment 78 is the method of any one of embodiments 68-77, comprising determining the methylation levels of the type-specific epigenetic target regions.

Embodiment 79 is the method of any one of embodiments 1-78, wherein the DNA molecules are amplified.

Embodiment 80 is the method of any one of embodiments 1-79, wherein the DNA molecules are amplified prior to the sequencing and/or
(a) prior to the selectively depleting the target nucleic acid comprising the wild-type sequence, the target nucleic acid comprising the converted nucleotide, or the target nucleic acid that does not comprise the converted nucleotide;
(b) prior to the enriching for one or more sets of target regions of DNA;
(c) prior to the partitioning the population of target nucleic acids into a plurality of subsamples; and/or
(d) prior to the subjecting the population of target nucleic acids to the conversion procedure that affects a first nucleobase of the DNA differently from a second nucleobase of the DNA.

Embodiment 81 is the method of any one embodiments 1-80, wherein the sequencing comprises sequencing the DNA in a modification-sensitive manner.

Embodiment 82 is the method of embodiment 81, wherein the sequencing in a modification-sensitive manner comprises long-read sequencing.

Embodiment 83 is the method of embodiment 81 or embodiment 82, wherein the sequencing in a modification-sensitive manner comprises nanopore sequencing.

Embodiment 84 is the method of embodiment 81, wherein the sequencing in a modification-sensitive manner comprises 5-letter or 6-letter sequencing.

Embodiment 85 is the method of any one embodiments 1-84, wherein the comprises next generation sequencing.

Embodiment 86 is the method of any one of embodiments 1-85, wherein the sequencing comprises generating a plurality of sequencing reads and mapping the plurality of sequencing reads to one or more reference sequences to generate mapped sequence reads.

Embodiment 87 is the method of embodiment 86, further comprising processing mapped sequence reads corresponding to the sequence-variable target region set and to the epigenetic target region set.

Embodiment 88 is the method of any one of embodiments 42-87, wherein the sequencing comprises sequencing at least a portion of the DNA, RNA, or cDNA generated from the RNA of at least the first and second subsamples in the same sequencing cell.

Embodiment 89 is the method of any one embodiments 1-88, wherein the population of nucleic acids comprises DNA.

Embodiment 90 is the method of embodiment 89, wherein the DNA is cell-free DNA.

Embodiment 91 is the method of any embodiments 1-90, wherein the population of target nucleic acids is from a blood sample and/or a tissue sample.

Embodiment 92 is the method of embodiment 91, wherein the blood sample is a whole blood sample, a plasma sample, a buffy coat sample, a leukapheresis sample, or a PBMC sample.

Embodiment 93 is the method of any one embodiments 1-92, wherein the population of target nucleotides is from a subject.

Embodiment 94 is the method of embodiment 93, wherein the subject is an animal.

Embodiment 95 is the method of embodiment 93 or embodiment 94, wherein the subject is a human.

Embodiment 96 is the method of any one of embodiments 93-95, wherein the subject has or is at risk of having a cancer.

Embodiment 97 is the method of any one embodiments 93-96, further comprising determining the presence or status of a cancer in the subject.

Embodiment 98 is the method of any one of embodiments 93-97, further comprising determining the likelihood that the subject has an infection.

Embodiment 99 is the method of any one of embodiments 93-98, further comprising determining the likelihood that the subject has a transplant rejection.

In some embodiments, the results of the methods disclosed herein are used as an input to generate a report. The report may be in a paper or electronic format. For example, the true methylation status of cytosines or variants, as obtained by the methods disclosed herein, or information derived therefrom, can be displayed directly in such a report. Alternatively or additionally, diagnostic information or therapeutic recommendations which are at least in part based on the methods disclosed herein can be included in the report.

The various steps of the methods disclosed herein may be carried out at the same or different times, in the same or different geographical locations, e.g. countries, and/or by the same or different people.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

FIG. 1 illustrates an exemplary workflow according to certain embodiments of the disclosure. The figure illustrates a method according to the disclosure, in which Y-shaped adapters comprising a restriction enzyme cleavage site (e.g., a BpsQI recognition site) and a universal primer binding site are ligated to a population of target DNA molecules, followed by contacting the population of target nucleic acids with an oligonucleotide probe. The oligonucleotide probe preferentially forms a substrate for extension with the wild-type sequence relative to the variant sequence, and the oligonucleotide probe is extended, producing a partially double-stranded target nucleic acid comprising the wild-type sequence. The partially double-stranded target nucleic acid comprising the wild-type sequence is selectively digested using a restriction enzyme that recognizes the cleavage site, and the population of target nucleic acids is then amplified and sequenced.

FIG. 2 illustrates an exemplary workflow according to certain embodiments of the disclosure. The figure illustrates a method according to the disclosure, in which cDNA is produced from a population of target RNA molecules, and Y-shaped adapters comprising a restriction enzyme cleavage site (e.g., a BpsQI recognition site) and a universal primer binding site are ligated to the cDNAs, followed by contacting the cDNAs with an oligonucleotide probe. The oligonucleotide probe preferentially forms a substrate for extension with the wild-type sequence present in the cDNAs relative to the variant sequence, and the oligonucleotide probe is extended, producing partially double-stranded cDNAs comprising the wild-type sequence. The partially double-stranded cDNAs comprising the wild-type sequence are selectively digested using a restriction enzyme that recognizes the cleavage site, and the cDNAs are then amplified and sequenced.

FIG. 3 illustrates various points in an exemplary workflow according to certain embodiments of the disclosure, at which a step of selectively depleting a target nucleic acid comprising a wild-type sequence (or selectively depleting a target nucleic acid comprising a converted nucleotide, or selectively depleting a target nucleic acid that does not comprise a converted nucleotide) may occur.

FIG. 4 is a schematic diagram of an example of a system suitable for use with some embodiments of the disclosure.

### DETAILED DESCRIPTION OF CERTAIN EMBODIMENTS

Reference will now be made in detail to certain embodiments of the invention. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids, reference to “a cell” includes a plurality of cells, and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes and are not to be construed as limiting the disclosed subject matter in any way. In the event that any document or other material incorporated by reference contradicts any explicit content of this specification, including definitions, this specification controls.

### I. Definitions

As used herein, “Buffy coat” refers to the portion of a blood (such as whole blood) or bone marrow sample that contains all or most of the white blood cells and platelets of the sample. The buffy coat fraction of a sample can be prepared from the sample using centrifugation, which separates sample components by density. For example, following centrifugation of a whole blood sample, the buffy coat fraction is situated between the plasma and erythrocyte (red blood cell) layers. The buffy coat can contain both mononuclear (e.g., T cells, B cells, NK cells, dendritic cells, and monocytes) and polymorphonuclear (e.g., granulocytes such as neutrophils and eosinophils) white blood cells.

“Cell-free DNA,” “cfDNA molecules,” or simply “cfDNA” include DNA molecules that naturally occur in a subject in extracellular form (e.g., in blood, serum, plasma, or other bodily fluids such as lymph, cerebrospinal fluid, urine, or sputum). While the cfDNA previously existed in a cell or cells in a large complex biological organism, e.g., a mammal, it has undergone release from the cell(s) into a fluid found in the organism, and may be obtained from a sample of the fluid without the need to perform an in vitro cell lysis step. cfDNA molecules may occur as DNA fragments.

As used herein, a modification or other feature is present in “a greater proportion” in a first sample or population of nucleic acid than in a second sample or population when the fraction of nucleotides with the modification or other feature is higher in the first sample or population than in the second population. For example, if in a first sample, one tenth of the nucleotides are mC, and in a second sample, one twentieth of the nucleotides are mC, then the first sample comprises the cytosine modification of 5-methylation in a greater proportion than the second sample.

As used herein, “leukapheresis” refers to a procedure in which white blood cells (leukocytes) are isolated from a sample of blood collected from a subject. Leukapheresis may be performed, e.g., obtain cells for research, diagnostic, prognostic, or monitoring purposes, such as those described herein. Thus, as used herein, a “leukapheresis sample” refers to a sample comprising leukocytes collected from a subject using leukapheresis.

As used herein, “peripheral blood mononuclear cells” or “PBMCs” refers to immune cells having a single, round nucleus that originate in bone marrow and are found in the peripheral circulation. Such cells include, e.g., lymphocytes (T cells, B cells, and NK cells) as well as monocytes, and are isolated from blood samples (such as from a whole blood sample collected from a subject) using density gradient centrifugation.

As used herein, “base pairing specificity” refers to the standard DNA base (A, C, G, or T) for which a given base most preferentially pairs. For example, unmodified cytosine and 5-methylcytosine have the same base pairing specificity (i.e., specificity for G) whereas uracil and cytosine have different base pairing specificity because uracil has base pairing specificity for A while cytosine has base pairing specificity for G. The ability of uracil to form a wobble pair with G is irrelevant because uracil nonetheless most preferentially pairs with A among the four standard DNA bases.

“Enriching” or “capturing” one or more target nucleic acids or one or more nucleic acids comprising at least one target region refers to preferentially isolating or separating the one or more target nucleic acids or one or more nucleic acids comprising at least one target region from non-target nucleic acids or from nucleic acids that do not comprise at least one target region.

An “enriched set” or “captured set” of nucleic acids or “enriched” or “captured” nucleic acids refers to nucleic acids that have undergone capture.

As used herein, a “capture moiety” is a molecule that allows affinity separation of molecules, such as nucleic acids, linked to the capture moiety from molecules lacking the capture moiety. Exemplary capture moieties include biotin, which allows affinity separation by binding to streptavidin linked or linkable to a solid phase or an oligonucleotide, which allows affinity separation through binding to a complementary oligonucleotide linked or linkable to a solid phase.

As used herein, a “cell type” is a set of cells having a shared characteristic. For example, cell types can include cells of different origins, differentiation types, different activation types, or any combination of different origins, different differentiation types, and different activation types. Indeed, differentiation status and activation status can overlap and often change together in a given cell, such as an immune cell or a cancer cell. For example, activation of an immune cell may induce differentiation of the cell. In some embodiments, cell types may be distinguished based on characteristics such as one or more cell surface markers, a genetic signature (such as expression (or expression level) of a particular gene or set of genes), and/or an epigenetic signature, such as regions of DNA hypermethylation or hypomethylation.

As used herein, a “cell cluster” or “cluster” is a plurality of related cell types, e.g., immune cell types, tissue-specific cell types, and/or cancer cell types. In some embodiments, the cell types within a cluster have similar DNA methylation profiles, e.g., in a plurality of hypermethylation variable target regions and/or hypomethylation variable target regions.

A “converted nucleobase” is a nucleobase having an altered base pairing specificity, wherein the original base pairing specificity of the nucleobase was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. As used herein, a “converted sample” is a sample comprising DNA comprising at least one converted nucleobase.

As used herein, an “oligonucleotide probe” refers to a single-stranded nucleic acid molecule (such as a DNA molecule or an RNA molecule) that can hybridize to a complementary nucleic acid sequence (e.g., a target sequence) within a target nucleic acid. An oligonucleotide probe may be 10 to 50 bases in length and may be synthesized as a specified base sequence.

“Specifically binds” in the context of a primer, a probe, or other oligonucleotide and a target sequence (e.g., a nucleic acid comprising a sequence that is partially or completely complementary to the primer, probe, or other oligonucleotide) means that under appropriate hybridization conditions, the primer, probe, or other oligonucleotide hybridizes to its target sequence, or replicates thereof, to form a stable hybrid, while at the same time formation of stable non-target hybrids is minimized. Thus, a primer, probe, or other oligonucleotide hybridizes to a target sequence or replicate thereof to a sufficiently greater extent than to a non-target sequence, to ultimately enable enrichment or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2^{nd} ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein). An oligonucleotide probe can “preferentially form a substrate for extension with,” e.g., a target nucleic acid comprising a wild-type sequence relative to a target nucleic acid comprising a variant sequence, a target nucleic acid that comprises a converted nucleotide relative to a target nucleic acid that does not comprise the converted nucleotide, or a target nucleic acid that does not comprise a converted nucleotide relative to a target nucleic acid that comprises the converted nucleotide. For example, an oligonucleotide probe that preferentially forms a substrate for extension with a target nucleic acid comprising a wild-type sequence relative to a target nucleic acid comprising a variant sequence may be at least 10 times, at least 15 times, at least 20 times, at least 25 times, at least 50 times, at least 100 times, at least 500 times, or at least 1000 times more likely to bind the target nucleic acid comprising the wild-type sequence relative to the target nucleic acid comprising the variant sequence.

A “target region” refers to a genomic locus targeted for identification and/or capture, for example, by using probes (e.g., through sequence complementarity). A “target region set” or “set of target regions” refers to a plurality of genomic loci targeted for identification and/or capture, for example, by using a set of probes (e.g., through sequence complementarity). A “target region set” can comprise regions that share at least one common feature. In some embodiments, a target region set is identified by the at least one common feature. For example, a hypermethylation variable target region set comprises regions of DNA that are hypermethylated.

“Sequence-variable target regions” refer to target regions that may exhibit changes in sequence such as nucleotide substitutions (i.e., single nucleotide variations), insertions, deletions, or gene fusions, or transpositions in neoplastic cells (e.g., tumor cells and cancer cells) relative to normal cells. A “sequence-variable target region set” refers to a set of sequence-variable target regions. In some embodiments, the sequence-variable target regions are target regions that may exhibit changes that affect less than or equal to 50 contiguous nucleotides, e.g., less than or equal to 40, 30, 20, 10, 5, 4, 3, 2, or 1 nucleotides.

“Epigenetic target regions” refers to target regions that may show sequence-independent differences in different cell or tissue types (e.g., different types of immune cells) or in abnormal cells, such as neoplastic cells (e.g., tumor cells and cancer cells), relative to normal cells; or that may show sequence-independent differences (i.e., in which there is no change to the nucleotide sequence, e.g., differences in methylation, nucleosome distribution, or other epigenetic features) in DNA, e.g., from different cell types or from subjects having cancer relative to DNA from healthy subjects. Examples of sequence-independent changes include, but are not limited to, changes in methylation (increases or decreases), nucleosome distribution, fragmentation patterns, CCCTC-binding factor (“CTCF”) binding, transcription start sites (e.g., with respect to any one of more of binding of RNA polymerase components, binding of regulatory proteins, fragmentation characteristics, and nucleosomal distribution), and regulatory protein binding regions. Epigenetic target region sets thus include, but are not limited to, hypermethylation variable target region sets, hypomethylation variable target region sets, and fragmentation variable target region sets, such as CTCF binding sites and transcription start sites.

For present purposes, loci susceptible to neoplasia-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed above than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions. An “epigenetic target region set” is a set of epigenetic target regions.

As used herein, a “differentially methylated region” refers to a region of DNA having a detectably different degree of methylation in at least one cell or tissue type relative to the degree of methylation in the same region of DNA from at least one other cell or tissue type; or having a detectably different degree of methylation in at least one cell or tissue type obtained from a subject having a disease or disorder relative to the degree of methylation in the same region of DNA in the same cell or tissue type obtained from a healthy subject. In some embodiments, a differentially methylated region has a detectably higher degree of methylation (e.g., a hypermethylated region) in at least one cell or tissue type, such as at least one immune cell type, relative to the degree of methylation in the same region of DNA from at least one other cell or tissue type, such as other immune cell types, or from the same cell or tissue type from a healthy subject. In some embodiments, a differentially methylated region has a detectably lower degree of methylation (e.g., a hypomethylated region) in at least one cell or tissue type, such as at least one immune cell type, relative to the degree of methylation in the same region of DNA from at least one other cell or tissue type, such as other immune cell types, or from the same cell or tissue type from a healthy subject.

A nucleic acid is “produced by a tumor” if it originated from a tumor cell. Tumor cells are neoplastic cells that originated from a tumor, regardless of whether they remain in the tumor or become separated from the tumor (as in the cases, e.g., of metastatic cancer cells and circulating tumor cells). As used herein, “precancer” or a “precancerous condition” is an abnormality that has the potential to become cancer, wherein the potential to become cancer is greater than the potential if the abnormality was not present, i.e., was normal. Examples of precancer include but are not limited to adenomas, hyperplasias, metaplasias, dysplasias, benign neoplasias (benign tumors), premalignant carcinoma in situ, and polyps. It should be noted that certain types of carcinoma in situ are recognized in the field as cancerous, e.g., Stage 0 cancer, as opposed to premalignant.

The term “methylation” or “DNA methylation” refers to addition of a methyl group to a nucleobase in a nucleic acid molecule. In some embodiments, methylation refers to addition of a methyl group to a cytosine at a CpG site (cytosine-phosphate-guanine site (i.e., a cytosine followed by a guanine in a 5′→3′ direction of the nucleic acid sequence)). In some embodiments, DNA methylation refers to addition of a methyl group to adenine, such as in N6-methyladenine. In some embodiments, DNA methylation is 5-methylation (modification of the 5^{th} carbon of the 6-carbon ring of cytosine). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3^{rd} carbon of the 6-carbon ring of cytosine). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

The term “hypermethylation” refers to an increased level or degree of methylation of nucleic acid molecule(s) relative to the other nucleic acid molecules comprising the same genetic information within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues.

The term “hypomethylation” refers to a decreased level or degree of methylation of nucleic acid molecule(s) relative to the other nucleic acid molecules comprising the same genetic information within a population (e.g., sample) of nucleic acid molecules. In some embodiments, hypomethylated DNA includes unmethylated DNA molecules. In some embodiments, hypomethylated DNA can include DNA molecules comprising 0 methylated residues, at most 1 methylated residue, at most 2 methylated residues, at most 3 methylated residues, at most 4 methylated residues, or at most 5 methylated residues.

The terms “agent that recognizes a modified nucleobase in DNA,” such as an “agent that recognizes a modified cytosine in DNA” refers to a molecule or reagent that binds to or detects one or more modified nucleobases in DNA, such as methyl cytosine. A “modified nucleobase” is a nucleobase that comprises a difference in chemical structure from an unmodified nucleobase. In the case of DNA, an unmodified nucleobase is adenine, cytosine, guanine, or thymine. In some embodiments, a modified nucleobase is a modified cytosine. In some embodiments, a modified nucleobase is a methylated nucleobase. In some embodiments, a modified cytosine is a methyl cytosine, e.g., a 5-methyl cytosine. In such embodiments, the cytosine modification is a methyl. Agents that recognize a methyl cytosine in DNA include but are not limited to “methyl binding reagents,” which refer herein to reagents that bind to a methyl cytosine. Methyl binding reagents include but are not limited to methyl binding domains (MBDs) and methyl binding proteins (MBPs) and antibodies specific for methyl cytosine. In some embodiments, such antibodies bind to 5-methyl cytosine in DNA. In some such embodiments, the DNA may be single-stranded or double-stranded. Suitable agents include agents that recognize modified nucleotides in double-stranded DNA, single-stranded DNA, and both double-stranded and single-stranded DNA.

The term “epigenetic status” refers to a certain level or extent of a sequence-independent variable that may be present in a DNA sequence. In some embodiments, the epigenetic status of a DNA sequence refers to the extent or level of methylation, nucleosome distribution, cfDNA fragmentation pattern, CCCTC-binding factor (“CTCF”) binding, transcription start site, or regulatory protein binding region of the sequence. Epigenetic statuses thus include, but are not limited to, hypermethylation, hypomethylation, and the presence of absence of CTCF binding sites or transcription start sites. The epigenetic status of a sequence may be a “reference epigenetic status” that can be used for comparison to the epigenetic status of the corresponding sequence in other DNA molecules. An example of a reference epigenetic status is a status that is prevalent in samples obtained from healthy subjects and is not associated with cancer.

As used herein, “methylation status” refers to the presence or absence of a methyl group on a DNA nucleobase (e.g. cytosine) at a particular genomic position in a nucleic acid, the degree of methylation of a nucleic acid (e.g., high, low, intermediate, or unmethylated), or the number of nucleotides methylated in a particular nucleic acid molecule. A nucleic acid “in methylated form” means that it comprises a sequence containing a methylated DNA nucleobase, e.g., a methylated cytosine in a CpG dinucleotide.

As used herein, the terms “neoplasm” and “tumor” are used interchangeably. They refer to abnormal growth of cells in a subject. A neoplasm or tumor can be benign, potentially malignant, or malignant. A malignant tumor is a referred to as a cancer or a cancerous tumor.

As used herein, “nucleic acid tag” refers to a short nucleic acid (e.g., less than about 500 nucleotides, about 100 nucleotides, about 50 nucleotides, or about 10 nucleotides in length), used to distinguish nucleic acids from different samples (e.g., representing a sample index), distinguish nucleic acids from different partitions (e.g., representing a partition tag) or different nucleic acid molecules in the same sample (e.g., representing a molecular barcode), of different types, or which have undergone different processing. The nucleic acid tag comprises a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples comprising nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g. molecular identifier, sample identifier). Additionally, or alternatively, nucleic acid tags can be used as molecular identifiers (e.g., to distinguish between different molecules or amplicons of different parent molecules in the same sample or sub-sample). This includes, for example, uniquely tagging different nucleic acid molecules in a given sample, or non-uniquely tagging such molecules. In the case of non-unique tagging applications, a limited number of tags (i.e., molecular barcodes) may be used to tag each nucleic acid molecule such that different molecules can be distinguished based on their endogenous sequence information (for example, start and/or stop positions where they map to a selected reference genome, a sub-sequence of one or both ends of a sequence, and/or length of a sequence) in combination with at least one molecular barcode. Typically, a sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode.

As used herein, “partitioning” refers to physically separating, sorting, and/or fractionating a mixture of nucleic acid molecules in a sample into a plurality of subsamples or subpopulations of nucleic acids based on a characteristic of the nucleic acid molecules. A sample or population may be partitioned into one or more partitioned subsamples or subpopulations based on a characteristic that is indicative of a genetic or epigenetic change or a disease state. The partitioning can be physical partitioning of molecules. Partitioning can involve separating the nucleic acid molecules into groups or sets based on the level of epigenetic feature (for e.g., methylation). For example, the nucleic acid molecules can be partitioned based on the level of methylation of the nucleic acid molecules. Stated differently, partitioning may include physically partitioning nucleic acid molecules based on the presence or absence of one or more methylated nucleobases. In some embodiments, the methods and systems used for partitioning may be found in PCT Patent Application No. PCT/US2017/068329, which is hereby incorporated by reference in its entirety.

As used herein, “partitioned set” or “partition” refers to a set of nucleic acid molecules partitioned into a set or group based on the differential binding affinity of the nucleic acid molecules or proteins associated with the nucleic acid molecules to a binding agent. A partitioned set may also be referred to as a subsample. The binding agent binds preferentially to the nucleic acid molecules comprising nucleotides with epigenetic modification. For example, if the epigenetic modification is methylation, the binding agent can be a methyl binding domain (MBD) protein. In some embodiments, a partitioned set can comprise nucleic acid molecules belonging to a particular level or degree of epigenetic feature (for e.g., methylation). For example, the nucleic acid molecules can be partitioned into three sets—one set for highly methylated nucleic acid molecules (first subsample, hyper partition, hyper partitioned set or hypermethylated partitioned set), a second set for low methylated nucleic acid molecules (second subsample, hypo partition, hypo partitioned set or hypomethylated partitioned set), and a third set for intermediate methylated nucleic acid molecules (third subsample, intermediate partitioned set, intermediately methylated partitioned set, residual partition, or residual partitioned set). In another example, the nucleic acid molecules can be partitioned based on the number of methylated nucleotides—one partitioned set can have nucleic acid molecules with nine methylated nucleotides, and another partitioned set can have unmethylated nucleic acid molecules (zero methylated nucleotides).

As used herein, a “restriction endonuclease” refers to an enzyme that can cleave a double stranded nucleic acid molecule (such as a double stranded DNA or cDNA) at a specific recognition site (a particular nucleotide sequence recognized by the restriction endonuclease, also referred to herein as a “cleavage site”) in the double stranded DNA or cDNA molecule. Exemplary restriction endonucleases of use herein include, but are not limited to, BspQI, AflIII, BsiHKAI, BtrI, MaeII, and SduI. In some embodiments, the restriction endonuclease is BspQI and claves a double-stranded nucleic acid at a 5′-GCTCTTCN-3′ recognition site.

As used herein, “sample” means anything capable of being analyzed by the methods and/or systems disclosed herein.

As used herein, “sequencing” refers to any of a number of technologies used to determine the sequence (e.g., the identity and order of monomer units) of a biomolecule, e.g., a nucleic acid such as DNA or RNA. Examples of sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, long-read sequencing, nanopore sequencing, 454 sequencing, Solexa Genome Analyzer sequencing, SOLiD™ sequencing, MS-PET sequencing, and a combination thereof. In some embodiments, sequencing can be performed by a gene analyzer such as, for example, gene analyzers commercially available from Illumina, Inc., Pacific Biosciences, Inc., or Applied Biosystems/Thermo Fisher Scientific, among many others.

As used herein, “next-generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next-generation sequencing techniques include, but are not limited to, sequencing by synthesis, sequencing by ligation, and sequencing by hybridization. In some embodiments, next-generation sequencing includes the use of instruments capable of sequencing single molecules. Example of commercially available instruments for performing next-generation sequencing include, but are not limited to, NextSeq, HiSeq, NovaSeq, MiSeq, Ion PGM and Ion GeneStudio S5.

As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual in need of therapy or suspected of needing therapy. The terms “individual” or “patient” are intended to be interchangeable with “subject”. For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

As used herein, the phrase “non-complementary restriction endonuclease cleavage site” refers to a restriction endonuclease cleavage site in a first strand of a double-stranded nucleic acid in which the second strand comprises at least one nucleotide that is not complementary to the corresponding nucleotide of the first strand. Non-complementary restriction endonuclease cleavage sites can comprise 1, 2, 3, 4, 5, 6, or more than 6 nucleotides that are not complementary to one another in this fashion. For example, in some embodiments of a double-stranded nucleic acid comprising 5′ and 3′ adapters on both strands, a 5′ adapter and a 3′ adapter comprise “non-complementary restriction endonuclease cleavage sites” when the cleavage site in the 5′ adapter comprises at least one nucleotide that is not complementary to the paired nucleotide of the 3′ adapter when the nucleic acids are aligned antiparallel to one another. Restriction endonuclease cleavage sites of use herein include cleavage sites that are recognized by a restriction endonuclease that cleaves a double-stranded nucleic acid comprising the cleavage site. Thus, in some embodiments, non-complementary restriction endonuclease cleavage sites, such as non-complementary restriction cleavage sites in otherwise complementary sense and antisense strands of a nucleic acid, may comprise cleavage sites that are recognized by different restriction endonucleases. Non-complementary restriction endonuclease cleavage sites can thus comprise a sense strand cleavage site that is longer or shorter than the cleavage site in the antisense strand.

As used herein, “substantially” refers to a range of numerical values (e.g., values within 5-10% of the specified vale) that one of ordinary skill in the art would consider equivalent to the recited value (e.g., having the same function or result). For example, “substantially” can include values greater than or equal to 90% of a specified value. Substantially does not require 100% (such as exactly 100% of the target nucleic acids of a population of target nucleic acids), but can include an amount greater than or equal to 90%, such as an amount greater than or equal to any one of 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% (such as 90%-100% of the target nucleic acids of a population of target nucleic acids).

“Mutation” refers to a variation from a known reference sequence and includes mutations such as, for example, single nucleotide variants (SNVs) and insertions or deletions (indels). A mutation can be a germline or somatic mutation. In some embodiments, a reference sequence for purposes of comparison is a wildtype genomic sequence of the species of the subject providing a test sample, typically the human genome.

A “X₁nnnX₂ mutation” in a specified polypeptide as used herein, where X₁ and X₂ are amino acids and nnn is a position in an amino acid sequence, refers to a substitution in the polypeptide of amino acid X₁ present at position nnn of the full-length wild-type polypeptide with amino acid X₂. The polypeptide is the human polypeptide unless indicated otherwise. The polypeptide comprising the X₁nnnX₂ mutation may, but does not necessarily, comprise additional differences from the wild-type sequence, including but not limited to truncations and deletions as well as other substitutions. For example, a “T1372S mutation” in TET2 refers to a substitution in a TET2 enzyme of the threonine present at position 1372 of the full-length wild-type human TET2 enzyme with a serine. Position 1372 of wild-type human TET2 aligns to position 258 and 248, respectively, of the truncated TET2 sequences disclosed as SEQ ID NOs: 23 and 24 of U.S. Pat. No. 10,961,525. The immediate wild-type sequence context of position 1372 of human TET2 is FSGVTACLD (SEQ ID NO: 13) where the T is at position 1372. Thus, a TET2 enzyme comprising a T1372S mutation may comprise the sequence FSGVSACLD (SEQ ID NO: 14) or optionally a variant of SEQ ID NO: 14 in which at least 5, 6, 7, or 8 positions match SEQ ID NO: 14 including position 5. Similarly, a “V1900X₂ mutation” where X₂ is A, C, G, I, or P in TET2 refers to a substitution in a TET2 enzyme of the valine present at position 1900 of the full-length wild-type human TET2 enzyme with an alanine, cysteine, glycine, isoleucine, or proline.

“Or” is used in the inclusive sense, i.e., equivalent to “and/or,” unless the context requires otherwise.

### II. Exemplary Methods

### A. Overview

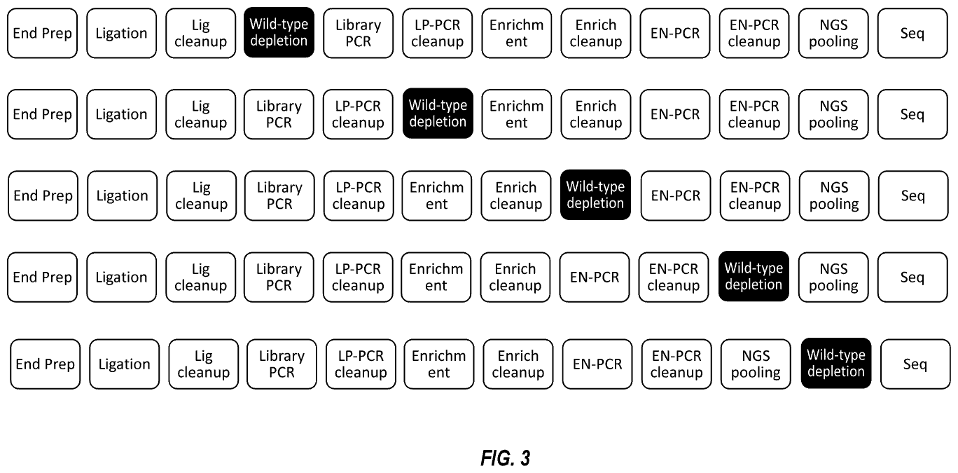

Cancer formation and progression may arise from both genetic modification and epigenetic features of DNA. However, assaying specific changes related to cancer that occur at very low frequencies, such as in early-stage cancers and pre-cancers, requires ultra-deep sequencing and/or enrichment, which can substantially increase assay costs. Some methods are available that allow for non-desired sequence depletion in RNA-seq workflows (such as for depletion of ribosomal RNA in a sample, See, e.g., US Patent Application No. 2015/0299767 A1). However, such methods are not designed to deplete targeted abundant wildtype sequences to enrich for sequence variants in detection assays (such as assays that can detect sequence variants in a liquid biopsy sample from a subject). Accordingly, the present disclosure provides methods and systems for selectively depleting a target nucleic acid comprising a wild-type sequence, a target nucleic acid comprising a converted nucleotide, or a target nucleic acid that does not comprise a converted nucleotide. Such methods allow for selective depletion of more abundant wild-type sequences (including sequences comprising wild-type epigenetic modifications) in target nucleic acids, such as in DNA or RNA (such as in cell-free DNA or cell-free RNA from a blood sample from a subject), thereby increasing the relative proportions of low-frequency nucleic acids comprising one or more variants (i.e., enriching for nucleic acids comprising the one or more variants) at a given locus as compared to nucleic acids comprising the wild-type sequence at the same locus. The disclosed methods may therefore reduce the sequencing depth required to detect and analyze the one or more variants, which can reduce sequencing and analysis costs. FIG. 3 illustrates various exemplary workflows according to certain embodiments of the disclosure.

In some embodiments of the disclosed methods, the population of target nucleic acids comprises DNA or cDNA. In some embodiments, a method of selectively depleting a target nucleic acid comprising a wild-type sequence comprises contacting the population of target nucleic acids with an oligonucleotide probe, wherein the population of target nucleic acids comprises a target nucleic acid comprising a wild-type sequence and comprises or is suspected of comprising a target nucleic acid comprising a variant sequence, and the oligonucleotide probe preferentially forms a substrate for extension with the wild-type sequence relative to the variant sequence. In such embodiments, extending the oligonucleotide probe produces a partially double-stranded target nucleic acid comprising the wild-type sequence. The partially double-stranded target nucleic acid comprising the wild-type sequence is selectively digested, thereby selectively depleting the target nucleic acid comprising the wild-type sequence.

In other embodiments, a method of selectively depleting a target nucleic acid that comprises a converted nucleotide comprises subjecting a population of target nucleic acids from a subject to a conversion procedure that affects a first nucleobase of the target nucleic acids differently from a second nucleobase of the target nucleic acids, wherein the first nucleobase is a modified or unmodified nucleobase, the second nucleobase is a modified or unmodified nucleobase different from the first nucleobase, and the first nucleobase and the second nucleobase have the same base pairing specificity, thereby providing a population of converted target nucleic acids. In such embodiments, the population of converted target nucleic acids is contacted with an oligonucleotide probe, wherein the oligonucleotide probe preferentially forms a substrate for extension with a target nucleic acid that comprises a converted nucleotide relative to a target nucleic acid that does not comprise the converted nucleotide. Extending the oligonucleotide probe produces a partially double-stranded target nucleic acid that comprises the converted nucleotide. The partially double-stranded target nucleic acid that comprises the converted nucleotide is then selectively digested, thereby selectively depleting the target nucleic acid that comprises the converted nucleotide.

In still other embodiments, a method of selectively depleting a target nucleic acid that does not comprise a converted nucleotide comprises subjecting a population of target nucleic acids to a conversion procedure that affects a first nucleobase of the target nucleic acids differently from a second nucleobase of the target nucleic acids, wherein the first nucleobase is a modified or unmodified nucleobase, the second nucleobase is a modified or unmodified nucleobase different from the first nucleobase, and the first nucleobase and the second nucleobase have the same base pairing specificity, thereby providing a population of converted target nucleic acids. In such embodiments, the population of converted target nucleic acids is contacted with an oligonucleotide probe, wherein the oligonucleotide probe preferentially forms a substrate for extension with a target nucleic acid that does not comprise a converted nucleotide relative to a target nucleic acid that comprises the converted nucleotide. Extending the oligonucleotide probe produces a partially double-stranded target nucleic acid that does not comprise the converted nucleotide. The partially double-stranded target nucleic acid that does not comprise the converted nucleotide is then selectively digested, thereby selectively depleting the target nucleic acid that does not comprise the converted nucleotide.

Some embodiments of the disclosed method further comprise a step of sequencing the population of target nucleic acids (such as using a sequencing method as disclosed herein) after selectively digesting the partially double-stranded target nucleic acid comprising the wild-type sequence, the partially double-stranded target nucleic acid that comprises the converted nucleotide, or the partially double-stranded target nucleic acid that does not comprise the converted nucleotide.

In particular embodiments, a method of selectively depleting a target nucleic acid comprising a wild-type sequence comprises contacting a population of target nucleic acids with an oligonucleotide probe, wherein substantially each target nucleic acid of the population of target nucleic acids is single-stranded and comprises an adapter comprising a restriction enzyme cleavage site and a universal primer binding site. In such embodiments, the population of target nucleic acids comprises a target nucleic acid comprising a wild-type sequence and comprises or is suspected of comprising a target nucleic acid comprising a variant sequence, and the oligonucleotide probe preferentially forms a substrate for extension with the wild-type sequence relative to the variant sequence. Extending the oligonucleotide probe produces a partially double-stranded target nucleic acid comprising the wild-type sequence. The partially double-stranded target nucleic acid comprising the wild-type sequence is selectively digested, thereby producing a selectively digested population of target nucleic acids. The selectively digested population of target nucleic acids is then amplified exponentially, producing a population of amplified target nucleic acids, and sequenced.

In other particular embodiments, a method of selectively depleting a target nucleic acid that comprises a converted nucleotide comprises subjecting a population of target nucleic acids to a conversion procedure that affects a first nucleobase of the target nucleic acids differently from a second nucleobase of the target nucleic acids, wherein substantially each target nucleic acid of the population of target nucleic acids is single-stranded and comprises an adapter comprising a restriction enzyme cleavage site and a universal primer binding site. In such embodiments, the first nucleobase is a modified or unmodified nucleobase, the second nucleobase is a modified or unmodified nucleobase different from the first nucleobase, and the first nucleobase and the second nucleobase have the same base pairing specificity, thereby providing a population of converted target nucleic acids. The population of converted target nucleic acids is contacted with an oligonucleotide probe, wherein the oligonucleotide probe preferentially forms a substrate for extension with a target nucleic acid that comprises a converted nucleotide relative to a target nucleic acid that does not comprise the converted nucleotide. Extending the oligonucleotide probe produces a partially double-stranded target nucleic acid that comprises the converted nucleotide. The partially double-stranded target nucleic acid that comprises the converted nucleotide is selectively digested, thereby producing a selectively digested population of target nucleic acids. The selectively digested population of target nucleic acids is then amplified exponentially, producing a population of amplified target nucleic acids, and sequenced.

In still other particular embodiments, a method of selectively depleting a target nucleic acid that does not comprise a converted nucleotide comprises subjecting a population of target nucleic acids to a conversion procedure that affects a first nucleobase of the target nucleic acids differently from a second nucleobase of the target nucleic acids, wherein substantially each target nucleic acid of the population of target nucleic acids is single-stranded and comprises an adapter comprising a restriction enzyme cleavage site and a universal primer binding site. In such embodiments, the first nucleobase is a modified or unmodified nucleobase, the second nucleobase is a modified or unmodified nucleobase different from the first nucleobase, and the first nucleobase and the second nucleobase have the same base pairing specificity, thereby providing a population of converted target nucleic acids. The population of converted target nucleic acids is contacted with an oligonucleotide probe, wherein the oligonucleotide probe preferentially forms a substrate for extension with a target nucleic acid that does not comprise a converted nucleotide relative to a target nucleic acid that comprises the converted nucleotide. Extending the oligonucleotide probe produces a partially double-stranded target nucleic acid that does not comprise the converted nucleotide. The partially double-stranded target nucleic acid that does not comprise the converted nucleotide is selectively digested, thereby producing a selectively digested population of target nucleic acids. The selectively digested population of target nucleic acids is then amplified exponentially, producing a population of amplified target nucleic acids, and sequenced.

In some embodiments, extending the oligonucleotide probe does not produce a partially double-stranded target nucleic acid comprising the variant sequence. In other embodiments, the method selectively depletes a target nucleic acid that comprises the converted nucleotide and does not produce a partially double-stranded target nucleic acid that does not comprise the converted nucleotide. In yet other embodiments, the method selectively depletes the target nucleic acid that does not comprise the converted nucleotide and extending the oligonucleotide probe does not produce a partially double-stranded target nucleic acid that comprises the converted nucleotide.

In particular embodiments, the variant sequence is a single nucleotide variant (SNV), an insertion or deletion (indel), a translocation, a gene fusion, or an epigenetic modification, such as modification of cytosine (e.g., 5-methylcytosine, 5-hydroxymethylcytosine, and other more oxidized forms) and/or association of DNA with chromatin proteins and transcription factors. In embodiments wherein a variant sequence comprises a mutation, the mutation may be a germline or somatic mutation.

### B. Oligonucleotide Probes

Oligonucleotide probes of use herein can hybridize to a complementary nucleic acid sequence (e.g., a target sequence, also referred to herein as an oligonucleotide probe binding site) within a single-stranded target nucleic acid (such as a target nucleic acid comprising a wild-type sequence, a target nucleic acid comprising a converted nucleotide, or a target nucleic acid that does not comprise a converted nucleotide). Oligonucleotide probes are known in the art. See, e.g., Wittwer et al., Nucleic Acid Techniques, in Principles and Applications of Molecular Diagnostics, Elsevier, 2018, pages 47-86. An oligonucleotide probe may be 10 to 50 bases in length, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases in length. An oligonucleotide probe hybridized to a complementary single-stranded nucleic acid sequence (such as a target sequence within a target nucleic acid) can be extended in a 5′ to 3′ direction to produce a double stranded or a partially double-stranded nucleic acid. In some embodiments of the present disclosure, extension of a hybridized oligonucleotide probe produces a partially double stranded nucleic acid. In some embodiments, a 5′ adapter is downstream of an oligonucleotide probe binding site within the target nucleic acid.

In some embodiments of the disclosed methods, a first portion of an oligonucleotide probe is complementary to a portion of a target nucleic acid comprising a wild-type sequence, and is not complementary to a portion of the target nucleic acid comprising a variant sequence. In some embodiments, a second portion of the oligonucleotide probe is complementary to both a portion of the target nucleic acid comprising the wild-type sequence and a portion of the target nucleic acid comprising the variant sequence. The first portion of the oligonucleotide probe may be located 3′ to the second portion of the oligonucleotide probe.

In other embodiments, a first portion of an oligonucleotide probe is complementary to a portion of a target nucleic acid that does not comprise a converted nucleotide, and is not complementary to a portion of the target nucleic acid that comprises the converted nucleotide. In other embodiments, a first portion of an oligonucleotide probe is complementary to a portion of the target nucleic acid that comprises a converted nucleotide, and is not complementary to a portion of the target nucleic acid that does not comprise the converted nucleotide. In some embodiments, a second portion of the oligonucleotide probe is complementary to both a portion of the target nucleic acid that does not comprise the converted nucleotide and a portion of the target nucleic acid that comprises the converted nucleotide. In some embodiments, the converted nucleotide was generated by deamination of unmodified or modified cytosine (e.g., 5-methylcytosine or 5-hydroxymethylcytosine). In some embodiments, the converted nucleotide comprises a nucleobase that preferentially pairs with adenine, such as uracil, thymine, or a modified form of uracil or thymine (e.g., dihydrouracil or 5-hydroxymethyluracil). The first portion of the oligonucleotide probe may be located 3′ to the second portion of the oligonucleotide probe.

In certain embodiments, the first portion of the oligonucleotide probe comprises a 3′ end of the oligonucleotide probe, and/or the second portion of the oligonucleotide probe comprises a 5′ end of the oligonucleotide probe.

In some embodiments, the method selectively depletes a target nucleic acid that comprises the converted nucleotide and the converted nucleotide is bound by the 3′ nucleotide of the oligonucleotide probe. In other embodiments, the method selectively depletes the target nucleic acid that does not comprise the converted nucleotide and the converted nucleotide is not bound by the 3′ nucleotide of the oligonucleotide probe. In particular embodiments, the 3′ portion of the oligonucleotide probe comprises one, two, three, four, or more than four of the 3′-most nucleotides of the oligonucleotide probe. In other particular embodiments, the 3′ portion of the oligonucleotide probe comprises one, two, three, or four of the four 3′-most nucleotides of the probe. In other particular embodiments, the 3′ portion of the oligonucleotide probe comprises one, two, or three of the three 3′-most nucleotides of the probe. In other particular embodiments, the 3′ portion of the oligonucleotide probe comprises one or two of the two 3′-most nucleotides of the probe. In some embodiments, the 3′ portion of the oligonucleotide probe comprises the four 3′-most nucleotides of the probe. In some embodiments, the 3′ portion of the oligonucleotide probe comprises the three 3′-most nucleotides of the probe. In some embodiments, the 3′ portion of the oligonucleotide probe comprises the two 3′-most nucleotides of the probe. In some embodiments, the 3′ portion of the oligonucleotide probe comprises the 3′-most nucleotide of the probe.

In some embodiments, the variant sequence comprises a single nucleotide variant (SNV), and the oligonucleotide probe comprises a 3′ portion that is complementary to the target nucleic acid comprising the wild-type sequence and is not complementary to the target nucleic acid comprising the SNV. In other embodiments, the variant sequence comprises an RNA fusion, and the oligonucleotide probe comprises a 3′ portion that is complementary to the target nucleic acid comprising the wild-type sequence and is not complementary to the target nucleic acid comprising the gene fusion. In still other embodiments, the variant sequence comprises a translocation, and the oligonucleotide probe comprises a 3′ portion that is complementary to the target nucleic acid comprising the wild-type sequence and is not complementary to the target nucleic acid comprising the translocation. Put another way, in the cases of the gene fusion and translocation, the oligonucleotide probe has sequence complementary to nucleotides on both sides of the breakpoint in the wild-type sequence where the gene fusion or translocation occurred, such that the gene fusion or translocation renders the 3′ portion of the oligonucleotide probe non-complementary to the version of the sequence comprising the gene fusion or translocation.

In certain embodiments, the variant sequence comprises an epigenetic modification (such as a cytosine modification, such as a methylcytosine (e.g., 5-methylcytosine or 5-hydroxymethylcytosine)), and where the oligonucleotide probe comprises a 3′ portion that is complementary to the target nucleic acid comprising the converted nucleotide and is not complementary to the target nucleic acid that does not comprise the converted nucleotide. In other embodiments wherein the variant sequence is an epigenetic modification, the oligonucleotide probe comprises a 3′ portion that is complementary to the target nucleic acid that does not comprise the converted nucleotide and is not complementary to the target nucleic acid comprising the converted nucleotide.

In some embodiments, an oligonucleotide probe comprises one or more modifications that prevent or reduce cleavage of the oligonucleotide probe by a restriction endonuclease after hybridization of the probe to a target nucleic acid. For example, the probe may comprise uracil or modified cytosine residues (e.g., 5-mC, 5-hmC, 5-ghmC, 5-propynylcytosine, or the like), LNA nucleotides, ribonucleotides, and/or phosphorothioate linkages. In some embodiments, the probe comprises one or more locked nucleic acid (LNA) nucleotides, i.e., a nucleotide modified such that the 2′-hydroxyl group of the ribosyl sugar ring is linked to the 4′ carbon atom of the sugar ring, e.g., via a methylene group, thereby forming a 2′-C,4′-C-oxymethylene linkage. LNAs include, but are not limited to, α-L-LNA, and β-D-LNA.

In some embodiments, the population of nucleic acids is contacted with a second oligonucleotide probe complementary to the strand of the target nucleic acid opposite the strand to which the first oligonucleotide probe is complementary.

In some embodiments, extending the oligonucleotide probe comprises contacting the substrate for extension with a DNA polymerase that lacks proof-reading activity (e.g., Taq, Therminator, or Bst DNA Polymerase). In such embodiments, the DNA polymerase lacks 3′->5′ exonuclease activity. Use of such a DNA polymerase prevents excision of bases that are mismatched, such as one or more bases in the oligonucleotide probe (e.g., at the 3′ end thereof) that are non-complementary to a sequence in a target nucleic acid. In such embodiments, an oligonucleotide probe having a 3′ end that does not anneal to the target nucleic acid will not be extended, and the target nucleic acid will not be selectively digested.

### C. Selective Digestion of a Target Nucleic Acid

When the oligonucleotide probe forms a substrate for extension with the wild-type sequence, it can be extended to produce a partially double-stranded target nucleic acid comprising the wild-type sequence. In some embodiments of the disclosed methods, partially double-stranded target nucleic acids comprising a wild-type sequence, partially double-stranded target nucleic acids that comprise a converted nucleotide, or partially double-stranded target nucleic acids that do not comprise the converted nucleotide are selectively digested. In particular embodiments, selectively digesting the partially double-stranded target nucleic acid comprising the wild-type sequence, selectively digesting the partially double-stranded target nucleic acid that comprises the converted nucleotide, or selectively digesting the partially double-stranded target nucleic acid that does not comprise the converted nucleotide comprises contacting the population of target nucleic acids with at least one restriction endonuclease. Restriction endonucleases of use herein can cleave a double-stranded nucleic acid and/or a partially double stranded nucleic acid, and do not cleave a single-stranded nucleic acid.

In some embodiments, a 5′ adapter, or both a 5′ adapter and a 3′ adapter, of a target nucleic acid comprises at least one sequence that is targeted by the at least one restriction enzyme. As disclosed herein, the 5′ adapter is downstream of an oligonucleotide probe binding site within the target nucleic acid. Thus, in some embodiments, the at least one restriction endonuclease cleaves the partially double-stranded target nucleic acid at the at least one sequence (cleavage site) within the at least one adapter. In some embodiments, the oligonucleotide probe, or the first and the second oligonucleotide probes, do not comprise the cleavage site.

In particular embodiments, the at least one restriction endonuclease is BspQI, AflIII, BsiHKAI, BtrI, MaeII, or SduI. In some embodiments, the at least one restriction endonuclease is BspQI, and cleaves a double-stranded (or partially double stranded) nucleic acid at a 5′-GCTCTTCN-3′ cleavage site in an adapter.

In some embodiments of the disclosed methods, selectively digesting the partially double-stranded target nucleic acid comprising the wild-type sequence, the partially double-stranded target nucleic acid that comprises the converted nucleotide, or the partially double-stranded target nucleic acid that does not comprise the converted nucleotide results in substantially linear amplification of the selectively digested target nucleic acid. Meanwhile, undigested target nucleic acid comprising the variant sequence, the unconverted nucleotide, or the converted nucleotide, respectively, can be exponentially amplified. In some embodiments, a concentration of the target nucleic acid comprising the variant sequence is increased relative to a concentration of the target nucleic acid comprising the wild-type sequence. In other embodiments, a concentration of the target nucleic acid comprising the converted nucleotide is increased relative to a concentration of the target nucleic acid that does not comprise the converted nucleotide. In yet other embodiments, a concentration of the target nucleic acid that does not comprise the converted nucleotide is increased relative to a concentration of the target nucleic acid comprising the converted nucleotide. The increase in concentration can result from exponential amplification producing more amplicon than linear amplification.

In particular embodiments wherein a target nucleic acid comprising a wild-type sequence is selectively digested, a post-selective digestion ratio of the target nucleic acid comprising the variant sequence to the target nucleic acid comprising the wild-type sequence is at least 1500:1, 1000:1, 500:1, 100:1, 10:1, 5:1, 1:1, 2:1, 3:1, 4:1, 5:1, 10:1, 100:1, or 1000:1. In particular embodiments wherein a target nucleic acid comprising a converted nucleotide is selectively digested, a post-selective digestion ratio of the target nucleic acid comprising the unconverted nucleotide to the target nucleic acid comprising converted nucleotide is at least 1500:1, 1000:1, 500:1, 100:1, 10:1, 5:1, 1:1, 2:1, 3:1, 4:1, 5:1, 10:1, 100:1, or 1000:1. In particular embodiments wherein a target nucleic acid that does not comprise a converted nucleotide is selectively digested, a post-selective digestion ratio of the target nucleic acid comprising the converted nucleotide to the target nucleic acid comprising the unconverted nucleotide is at least 1500:1, 1000:1, 500:1, 100:1, 10:1, 5:1, 1:1, 2:1, 3:1, 4:1, 5:1, 10:1, 100:1, or 1000:1.

In some embodiments (e.g., wherein the selective depletion step occurs after an amplification step), a target nucleic acid may comprise a 5′ adapter and a 3′ adapter, wherein the 5′ and 3′ adapters comprise non-complementary restriction endonuclease cleavage sites. In some embodiments, the 5′ adapter comprises a first sequence that is targeted by a first restriction endonuclease and the 3′ adapter comprises a second sequence that is targeted by a second restriction endonuclease. In some embodiments, a restriction endonuclease cleavage site in a 5′ adapter comprises one, two, three, four, five, six, or more than six bases that are non-complementary to a restriction endonuclease cleavage site in a 3′ adapter. In such embodiments, use of different (mismatched) restriction endonuclease cleavage sites in the 5′ and 3′ adapters may avoid or reduce unwanted re-annealing (and subsequent digestion by a restriction endonuclease that recognizes the double-stranded cleavage site in the re-annealed adapters) of target nucleic acids that were not extended following the contacting with an oligonucleotide probe. In such embodiments, the selective digestion may comprise a first restriction enzyme that recognizes the first sequence and a second restriction enzyme that recognizes the second sequence.

In other embodiments (e.g., wherein the selective depletion step occurs after an amplification step), a restriction endonuclease cleavage site in one strand of an adapter (e.g., a 3′ adapter) attached to a target nucleic acid can comprise uracils in place of thymines and/or a modified base, such as a modified cytosine, such as a methylated or a hydroxymethylated cytosine. Most restriction endonucleases are not able to cleave nucleic acids at cleavage sites comprising uracils. See, e.g., Kisiala et al., Nucleic Acids Res. 2020, 48(12):6954-6969. Similarly, methylation-sensitive restriction enzymes (MSRE) are not able to cleave nucleic acids at cleavage sites comprising methylated cytosine residues. Thus, in some embodiments, if otherwise complementary target nucleic acids that were not extended (following the step of contacting the population of target nucleic acids with an oligonucleotide probe) re-anneal to one another, the presence of one or more uracil bases and/or one or more modified bases in the cleavage sites of the 3′ adapters may avoid or reduce unwanted digestion of the re-annealed nucleic acids by a restriction endonuclease (e.g., HpaII, BstUI, Hin6I, SsiI, and HpyCH4IV) that, in the absence of the one or more uracils and/or modified bases, would otherwise be capable of cleaving the double-stranded nucleic acids at the cleavage site in the re-annealed adapters. In some embodiments, the restriction endonuclease that, in the absence of the one or more uracils and/or modified bases, would otherwise be capable of cleaving the double-stranded nucleic acids at the cleavage site in the re-annealed adapters, is HpaII, BstUI, Hin6I, SsiI, or HpyCH4IV. In particular embodiments, the restriction endonuclease is HpaII. In other particular embodiments, the restriction endonuclease is BstUI. In other particular embodiments, the restriction endonuclease is Hin6I. In other particular embodiments, the restriction endonuclease is SsiI. In other particular embodiments, the restriction endonuclease is HpyCH4IV.

In other embodiments, a restriction endonuclease cleavage site in a 3′ adapter attached to a target nucleic acid can comprise a modified base, such as a modified cytosine, such as a methylated or a hemimethylated cytosine. In such embodiments, if complementary target nucleic acids that were not extended following the contacting with an oligonucleotide probe reanneal to one another, the presence of uracil bases and/or modified bases in the 3′ adapters may avoid or reduce unwanted digestion of the re-annealed nucleic acids by a restriction endonuclease that, without the uracils and/or modified bases, would otherwise be capable of recognizing the double-stranded cleavage site in the re-annealed adapters.

In other embodiments (e.g., wherein the selective depletion step occurs after an amplification step), the selective depletion step may further comprise contacting the population of target nucleic acids with a blocking oligonucleotide that anneals to a 5′ and/or a 3′ adapter of the target nucleic acids. In such embodiments, the blocking oligonucleotide comprises one or more modifications that prevent or reduce cleavage of the adapter by a restriction endonuclease that recognizes a double-stranded cleavage sequence in the adapter, and further may avoid or reduce unwanted re-annealing (and subsequent digestion by a restriction endonuclease that recognizes the double-stranded cleavage site in the re-annealed adapters) of target nucleic acids that were not extended following the contacting with an oligonucleotide probe. In such embodiments, extension of the annealed oligonucleotide probe can be performed using a strand-displacing polymerase that displaces the blocking oligonucleotide from the adapter and re-synthesizes the adapter to provide a double-stranded sequence that can be cleaved by the restriction endonuclease.

### D. RNA Extraction and Isolation

In some embodiments of the disclosed methods, the population of target nucleic acids comprises RNA and the method further comprises a cDNA synthesis step. RNA for use in the methods disclosed herein may be isolated from a blood sample or a sample comprising cells (such as a sample that includes immune and/or cancer-derived cells (e.g., a blood sample such as a whole blood sample, a buffy coat sample, a leukapheresis sample, or a peripheral blood PBMC sample)). General methods for RNA extraction and isolation (such as mRNA extraction and isolation) are known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). In particular, RNA isolation can be performed using a purification kit, buffer set, and protease(s) from commercial manufacturers, such as PreAnalytix GmbH or Qiagen, according to the manufacturer's instructions. For example, RNA can be extracted from whole blood samples using the PAXgene® Blood RNA Kit (PreAnalytix GmbH). Other commercially available RNA isolation kits include MasterPure™ Complete DNA and RNA Purification Kit (EPICENTRE®, Madison, WI), and Paraffin Block RNA Isolation Kit (Ambion, Inc.). Total RNA from tissue samples can be isolated using RNA Stat-60 (Tel-Test). RNA prepared from tumor tissue can be isolated, for example, by cesium chloride density gradient centrifugation.

E. cDNA Library Preparation

Following RNA extraction from a sample (such as a blood sample), a cDNA library is typically prepared in preparation for sequencing, e.g., as in RNA-Seq. In some embodiments, the cDNAs in a library, such as an RNA-Seq library, can comprise a cDNA insert flanked by adapter sequences, such as adapter sequences used for amplification and sequencing on a particular platform. Exemplary cDNA library preparation methods are discussed below; however, cDNA library preparation methods can vary depending on the RNA species under investigation, which can differ in size, sequence, structural features and abundance. One of ordinary skill in the art will be able to select cDNA library preparation methods suitable for cDNA library preparation using an RNA species of interest.

1. rRNA and/or Globin mRNA Depletion; Poly(A) Selection

Ribosomal RNAs (rRNAs) are the most abundant RNA species in most cells. Globin mRNA is also abundant in certain cell types found in the blood. Thus, some embodiments of the present disclosure comprise a step of ribosomal RNA (rRNA) depletion and/or a step of globin mRNA depletion. Such steps can be performed, e.g., following RNA extraction from a sample, and prior to a step of RNA fragmentation or cDNA fragmentation, prior to a step preparing cDNA from the RNA, prior to a step of ligating adapters to the cDNA, and prior to a sequencing step. In some embodiments, the methods include a step of rRNA depletion. In other embodiments, the methods include a step of globin mRNA depletion. In yet other embodiments, the methods disclosed herein include both a step of rRNA depletion and a step of globin mRNA depletion.

Any suitable rRNA depletion and/or globin mRNA depletion methods are of use in the present disclosure. One approach is to eliminate rRNAs uses sequence-specific probes that can hybridize to rRNAs (Hrdlickova et al., Wiley Interdiscip Rev RNA. 2017; 8(1):10.1002/wrna.1364). Unwanted rRNAs or their cDNAs are hybridized with biotinylated DNA or locked nucleic acid (LNA) probes, followed by depletion with streptavidin beads. Alternatively, rRNAs can be targeted by anti-sense DNA oligos and digested by RNase H, a method also known as probe-directed degradation (PDD). Another approach for rRNA reduction uses specific, not-so-random (NSR) primers that bind to the RNA molecules of interest during reverse transcription, thus avoiding reverse transcription of the rRNAs. For example, a method known as Ovation RNA-Seq (NuGen) uses hexamer or heptamer primers whose sequences are not present in rRNAs. In addition to sequence-based approaches, some methods take advantage of certain features of rRNAs for their elimination. The C₀T-hybridization method is based on heat denaturation, re-annealing, and selective degradation by a duplex-specific nuclease (DSN). Double-stranded cDNAs from abundant sequences are preferentially degraded because of their more rapid annealing kinetics compared to less abundant ones. Selective degradation has also been achieved using the enzyme terminator 5′-phosphate-dependent exonuclease (TEX), which recognizes RNA molecules with 5′-monophosphate, as with rRNAs and tRNAs. Further, commercial kits are available for rRNA and globin mRNA depletion, including, e.g., the Watchmaker Genomics RNA Library Prep Kit with Polaris Depletion.

Other embodiments of the present disclosure comprise a step of poly(A) selection. Such a step can be performed, e.g., following RNA extraction from a sample, and prior to a step of RNA fragmentation or cDNA fragmentation, prior to a step preparing cDNA from the RNA, prior to a step of ligating adapters to the cDNA, and prior to a sequencing step. In eukaryotic organisms, most protein coding RNAs (mRNAs) and many long noncoding RNAs (lncRNAs) (>200 nt) comprise a poly(A) tail (“polyadenylated RNAs”). The poly(A) tail may be used to enrich for polyadenylated RNAs from total cellular RNA, in which polyadenylated RNAs may account for approximately 1-5% of total cellular RNA (Hrdlickova et al., Wiley Interdiscip Rev RNA. 2017 8(1):10.1002/wrna.1364). Exemplary poly(A) selection methods include, but are not limited to, use of magnetic or cellulose beads coated with oligo-dT molecules. Alternatively, polyadenylated RNAs can be selected using oligo-dT priming for reverse transcription (RT). Poly(A) selection may be combined with globin mRNA depletion.

### 2. Fragmentation

In some embodiments, methods disclosed herein comprise fragmenting RNA isolated from a sample (such as RNA isolated from a sample comprising cells, such as a whole blood sample, a buffy coat sample, a leukapheresis sample, or a PBMC sample), such as following poly(A) selection or rRNA and/or globin mRNA depletion. RNA fragmentation methods can include physical fragmentation, chemical fragmentation, and/or enzymatic fragmentation. Physical fragmentation methods include, but are not limited to, acoustic or hydrodynamic shearing (such as sonication or point-sink shearing), needle shearing, and nebulization. Enzymatic fragmentation methods can include use of a ribonuclease (such as RNase III). RNA may also be fragmented using chemical shearing methods. Chemical fragmentation methods can include, but are not limited to, heat treatment of RNA in the presence of a divalent metal cation (such as magnesium or zinc). In some embodiments, the fragmenting provides RNA (such as mRNA) fragments of 25-400, 25-300, 25-200, 50-400, 50-300, 50-250, 50-200, 100-400, 100-300, 100-200, 125-400, 125-300, 125-200, 125-175, 150-400, 150-300, 200-400, 250-400, 300-400, 200-350, 200-300, 225-375, 250-350, or 275-325 base pairs in length.

Alternatively, non-fragmented RNAs can be reverse transcribed, and the resultant cDNA can be fragmented. cDNA fragmentation methods can include physical fragmentation, chemical fragmentation, and/or enzymatic fragmentation. Physical fragmentation methods include, but are not limited to, acoustic or hydrodynamic shearing (such as sonication or point-sink shearing), needle shearing, and nebulization. Enzymatic fragmentation methods can include use of a restriction endonuclease (such as a 4-cutter or 5-cutter restriction endonuclease, e.g., AluI, DpnI, Eco47I, HaeIII, HpaII, Mbo I, MseI, MspI, PspGI, RsaI, Sse9I, or TaqI), a non-specific nuclease (e.g., micrococcal nuclease), or a transposase (for example, when insertion of an adapter into a fragmented double-stranded cDNA molecule is desired). cDNA may also be fragmented using chemical shearing methods. Chemical fragmentation methods can include, but are not limited to, heat digestion of cDNA in the presence of a divalent metal cation (such as magnesium or zinc). In some embodiments, the fragmenting provides cDNA fragments of 25-400, 25-300, 25-200, 50-400, 50-300, 50-250, 50-200, 100-400, 100-300, 100-200, 125-400, 125-300, 125-200, 125-175, 150-400, 150-300, 200-400, 250-400, 300-400, 200-350, 200-300, 225-375, 250-350, or 275-325 base pairs in length.

3. cDNA Preparation

Some embodiments of the disclosed methods comprise preparing cDNA from RNA (such as RNA extracted from a blood sample), such as by reverse transcription of the RNA template into cDNA. Reverse transcription is generally followed by exponential amplification of the cDNA, e.g., in a PCR reaction. Two commonly used reverse transcriptases are avian myeloblastosis vims reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse transcribed using a Gene Amp RNA PCR kit (Perkin Elmer, Calif, USA), following the manufacturer's instructions. The derived cDNA can then be used as a template in the subsequent amplification (e.g., PCR) reaction. In some embodiments, RNA is converted to cDNA using random priming, followed by second strand synthesis, end repair, and optional A-tailing. Adapters comprising barcodes can then be ligated to the cDNA, which is then amplified.

Amplification is typically primed by primers that anneal or bind to primer binding sites in adapters flanking a cDNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence-based amplification, and self-sustained sequence-based replication.

Although a PCR step can use a variety of thermostable DNA-dependent DNA polymerases, it typically employs the Taq DNA polymerase. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

The primers used for the amplification are selected so as to amplify a unique segment of the gene of interest, such as RNA (such as mRNA) encoding a gene of a target gene set described herein. In some embodiments, expression of other genes is also detected, such as other known disease markers (such as known cancer markers) or housekeeping genes. Primers that can be used to amplify disease-related molecules are commercially available or can be designed and synthesized. In some examples, the primers specifically hybridize to a promoter or promoter region of a disease-related molecule. An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified cDNA from each segment is determined and compared to a standard curve to determine the amount of the target nucleic acid segment that was present in the sample prior to amplification. In some embodiments, the expression of a “housekeeping” gene or “internal control” can also be evaluated. These terms include any constitutively or globally expressed gene whose presence enables an assessment of mRNA levels provided herein. Such an assessment includes a determination of the overall constitutive level of gene transcription and a control for variations in RNA recovery. Exemplary housekeeping genes include tubulin, glyceraldehyde-3-phosphate-dehydrogenase (GAPDH), beta-actin, and 18S ribosomal RNA.

### F. Adapter Ligation or Addition; Tagging

In some embodiments, the disclosed methods comprise adding adapters to DNA (such as cDNA, cell-free DNA, or fragmented genomic DNA). In some embodiments, adapters are added to the DNA before or after subjecting the DNA to a procedure that affects a first nucleobase in the DNA differently from a second nucleobase in the DNA, such as after the subjecting. When adapters are added to the DNA before subjecting the DNA to a procedure that affects a first nucleobase in the DNA differently from a second nucleobase in the DNA, they may comprise nucleotides that are resistant to the procedure. For example, where the procedure comprises contacting the DNA with a deaminase, the adapters may comprise deaminase-resistant cytosines such as 5-ghmC or 5-propynyl cytosine. Similarly, where the procedure comprises contacting the DNA with bisulfite, the adapters may comprise bisulfite-resistant cytosines such as 5-mC or 5-hmC. In some embodiments, adapters may be added or to DNA concurrently with an amplification procedure, e.g., by providing the adapters in a 5′ portion of a primer (where PCR is used, this can be referred to as library prep-PCR or LP-PCR), before or after an amplification step. In some embodiments, adapters are added by other approaches. In some such methods, first adapters are added to the nucleic acids by ligation to the 3′ ends thereof, which may include ligation to single-stranded DNA. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified. In some embodiments, end repair of the DNA is performed prior to addition of adapters.

In some embodiments, the single-stranded DNA library preparation is performed in a one-step combined phosphorylation/ligation reaction, e.g., as described in Troll et al., BMC Genomics, 20:1023 (2019), available at https://doi.org/10.1186/s12864-019-6355-0. This method, called Single Reaction Single-stranded LibrarY (“SRSLY,”) can be performed without end-polishing. SRSLY may be useful for converting short and fragmented DNA molecules, e.g., cfDNA fragments, into sequencing libraries while retaining native lengths and ends. The SRSLY method can create sequencing libraries (e.g., Illumina sequencing libraries) from fragmented or degraded template (input) DNA. In particular embodiments, template DNA is first heat denatured and then immediately cold shocked to render the template DNA molecules single-stranded. The DNA can be maintained as single-stranded throughout the ligation reaction by the inclusion of a thermostable single-stranded binding protein (SSB). Next, the template DNA, which at this point can be single-stranded and coated with SSB, is placed in a phosphorylation/ligation dual reaction with directional dsDNA NGS adapters that contain single-stranded overhangs. Both the forward and reverse sequencing adapters can share similar structures but differ in which termini is unblocked in order to facilitate proper ligations. Both sequencing adapters can comprise a dsDNA portion and a single-stranded splint overhang of random nucleotides that occurs on the 3-prime terminus of the bottom strand of the forward adapter and the 5-prime terminus of the bottom strand of the reverse adapter. In this way, the forward adapter (e.g., (P5) Illumina adapter) can delivered to the 5-prime end of template molecules and the reverse adapter (e.g., (P7) Illumina adapter) is delivered to the 3-prime end of template molecules. Thus, the native polarity of input DNA molecules can be retained.

During the dual phosphorylation/ligation reaction, T4 Polynucleotide Kinase (PNK) can be used to prepare template DNA termini for ligation by phosphorylating 5-prime termini and dephosphorylating 3-prime termini. T4 PNK works on both ssDNA and dsDNA molecules and has no activity on the phosphorylation state of proteins. Simultaneously, the random nucleotides of the splint adapter can be annealed to the single-stranded template molecule. This creates a short, localized dsDNA molecule, enabling ligation of template to adapter with a ligase such as T4 DNA ligase, which has high ligation efficiency on dsDNA templates but low efficiency on ssDNA. After the single phosphorylation/ligation reaction is complete, the library DNA can be, e.g., purified and placed directly into standard NGS indexing PCR, compatible with both traditional single or dual index primers.

In some embodiments, following attachment of adapters, the nucleic acids are subject to amplification. The amplification can use, e.g., universal primers that recognize primer binding sites in the adapters.

In some embodiments, the DNA is linked at both ends to Y-shaped adapters including primer binding sites and tags. In some such embodiments, the DNA is amplified.

In embodiments of the disclosed methods, a target nucleic acid comprises a 5′ adapter, a 3′ adapter, or both a 5′ adapter and a 3′ adapter. In such embodiments, the 5′ adapter, or both the 5′ adapter and the 3′ adapter comprise at least one sequence that is recognized by at least one restriction enzyme, such as a restriction enzyme described elsewhere herein. In particular embodiments, the 5′ adapter is downstream of an oligonucleotide probe binding site within the target nucleic acid.

Tagging DNA molecules is a procedure in which a tag is attached to or associated with the DNA molecules. Such tags can be molecules, such as nucleic acids, containing information that indicates a feature of the molecule with which the tag is associated. Tags can allow one to differentiate molecules from which sequence reads originated. For example, molecules can bear a sample tag (which distinguishes molecules in one sample from those in a different sample) or a molecular tag/molecular barcode/barcode (which distinguishes different molecules from one another in both unique and non-unique tagging scenarios). For methods that involve a partitioning step, a partition tag (which distinguishes molecules in one partition from those in a different partition) may be included. In some embodiments, adapters added to DNA molecules comprise tags. In some such embodiments, the tag comprises one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, or molecular tags can be used such that the barcodes serve as a molecular tag through their individual sequences and also serve to identify the partition and/or sample to which they correspond based the set of which they are a member. Tags comprising barcodes can be incorporated into or otherwise joined to adapters. Tags can be incorporated by ligation, overlap extension PCR among other methods.

Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In some embodiments, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites. In some embodiments, adapters include the same primer binding site.

In certain embodiments of non-unique tagging, the number of different tags used can be sufficient that there is a very high likelihood (e.g., at least 99%, at least 99.9%, at least 99.99% or at least 99.999% that all molecules of a particular group bear a different tag. In some embodiments comprising barcode attachment, e.g., randomly, to both ends of a molecule, the combination of barcodes, together, constitutes a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene). In certain embodiments, the number of different tags used to uniquely identify a number of molecules, z, in a class can be between any of 2*z, 3*z, 4*z, 5*z 6*z, 7*z 8*z, 9*z, 10*z, 11 *z 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit).

For example, in a sample of about 5 ng to 30 ng of DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

Generally, assignment of unique or non-unique tags barcodes in reactions follows methods and systems described by US patent applications 20010053519, 20030152490, 20110160078, and U.S. Pat. Nos. 6,582,908 and 7,537,898 and 9,598,731. Tags can be linked to sample nucleic acids randomly or non-randomly.

In some embodiments, the tagged nucleic acids are sequenced after loading into a microwell plate. The microwell plate can have 96, 384, or 1536 microwells. In some cases, they are introduced at an expected ratio of unique tags to microwells. For example, the unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

In some embodiments, 20-50 different tags (e.g., barcodes) are ligated to both ends of target nucleic acids. For example 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

In some embodiments, two or more populations, samples, subsamples, or partitions are differentially tagged, such as partitioned subsamples and/or subsamples that are differentially degraded using one or more methylation-sensitive nucleases. Tags can be used to label the individual DNA populations so as to correlate the tag (or tags) with a specific population or partition. In some embodiments, a single tag can be used to label a specific population or partition. In some embodiments, multiple different tags can be used to label a specific population or partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108: 9530-9535 (2011), Kou et al., PLoS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after tagging, the molecules may be pooled for sequencing in a single run. In some embodiments, a sample tag is added to the molecules, e.g., in a step subsequent to addition of other tags and pooling. Sample tags can facilitate pooling material generated from multiple samples for sequencing in a single sequencing run.

In some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules based on one or more characteristics, the final tagged molecules in the library may no longer possess that characteristic. For example, while single stranded DNA molecules may be partitioned and/or tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a pooled DNA level. Tags are used to sort reads from different partitions. Analysis can include in silico analysis to determine genetic and epigenetic variation (one or more of methylation, chromatin structure, etc.) using sequence information, genomic coordinates length, coverage, and/or copy number.

G. Subjecting the DNA or a Subsample Thereof to a Procedure that Affects a First Nucleobase in the DNA Differently from a Second Nucleobase in the DNA

In some embodiments, methods disclosed herein comprise a step of subjecting DNA, or a subsample thereof, to a procedure that affects a first nucleobase in the DNA differently from a second nucleobase in the DNA, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, the procedure chemically converts the first or second nucleobase such that the base pairing specificity of the converted nucleobase is altered. In some embodiments, DNA is subjected to a procedure that affects a first nucleobase in the DNA differently from a second nucleobase in the DNA before library preparation using the DNA, before a first amplification of the DNA, before dividing the DNA into a plurality of subsamples, or any combination thereof. In certain embodiments, the DNA is subjected to the procedure before or after contacting the DNA with a methylation-sensitive nuclease.

In some embodiments, the procedure that affects a first nucleobase of the DNA differently from a second nucleobase of the DNA is performed prior to the sequencing and/or (a) prior to or after the selectively depleting the target nucleic acid comprising the wild-type sequence, the target nucleic acid comprising the converted nucleotide, or the target nucleic acid that does not comprise the converted nucleotide; (b) prior to the amplifying the selectively digested population of target nucleic acids; (c) prior to or after the partitioning the population of target nucleic acids into a plurality of subsamples; and/or (d) prior to or after a step of enriching for one or more sets of target regions of DNA.

In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, such as where one of the first and second nucleobases comprises mC and the other comprises hmC.

In some embodiments, the procedure that affects a first nucleobase in the DNA differently from a second nucleobase in the DNA comprises bisulfite conversion. Treatment with bisulfite converts unmodified cytosine and certain modified cytosine nucleotides (e.g. 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase comprises one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion, such as on a DNA sample as described herein, facilitates identifying positions containing mC or hmC using the sequence reads obtained from the exemplary sample. For an exemplary description of bisulfite conversion, see, e.g., Moss et al., Nat Commun. 2018; 9: 5068.

In some embodiments, the procedure that affects a first nucleobase in the DNA differently from a second nucleobase in the DNA comprises oxidative bisulfite (Ox-BS) conversion. This procedure first converts hmC to fC, which is bisulfite susceptible, followed by bisulfite conversion. Thus, when oxidative bisulfite conversion is used, the first nucleobase comprises one or more of unmodified cytosine, fC, caC, hmC, or other cytosine forms affected by bisulfite, and the second nucleobase comprises mC. Sequencing of Ox-BS converted DNA identifies positions that are read as cytosine as being mC positions. Meanwhile, positions that are read as T are identified as being T, hmC, or a bisulfite-susceptible form of C, such as unmodified cytosine, fC, or hmC. Performing Ox-BS conversion, such as on a DNA sample as described herein, thus facilitates identifying positions containing mC using the sequence reads obtained from the sample. For an exemplary description of oxidative bisulfite conversion, see, e.g., Booth et al., Science 2012; 336: 934-937.

In some embodiments, the procedure that affects a first nucleobase in the DNA differently from a second nucleobase in the DNA comprises Tet-assisted bisulfite (TAB) conversion. In TAB conversion, hmC is protected from conversion and mC is oxidized in advance of bisulfite treatment, so that positions originally occupied by mC are converted to U while positions originally occupied by hmC remain as a protected form of cytosine. For example, as described in Yu et al., Cell 2012; 149: 1368-80, β-glucosyl transferase can be used to protect hmC (forming 5-glucosylhydroxymethylcytosine (ghmC)), then a TET protein such as mTet1 can be used to convert mC to caC, and then bisulfite treatment can be used to convert C and caC to U while ghmC remains unaffected.

Alternatively, a carbamoyltransferase enzyme, such as 5-hydroxymethylcytosine carbamoyltransferase as described in Yang et al., Bio-protocol, 2023; 12 (17): e4496, can be used to protect hmC (by converting hmC to 5-carbamoyloxymethylcytosine (5cmC)), then a TET protein such as mTet1 or a TET2 comprising a T1372S mutation, can be used to convert mC to caC, and then bisulfite treatment can be used to convert C and caC to U while 5cmC remains unaffected. Thus, when TAB conversion is used, the first nucleobase comprises one or more of unmodified cytosine, fC, caC, mC, or other cytosine forms affected by bisulfite, and the second nucleobase comprises hmC. Sequencing of TAB-converted DNA identifies positions that are read as cytosine as being hmC positions. Meanwhile, positions that are read as T are identified as being T, mC, or a bisulfite-susceptible form of C, such as unmodified cytosine, fC, or caC. Performing TAB conversion, such as on a DNA sample as described herein, thus facilitates identifying positions containing hmC using the sequence reads obtained from the sample.

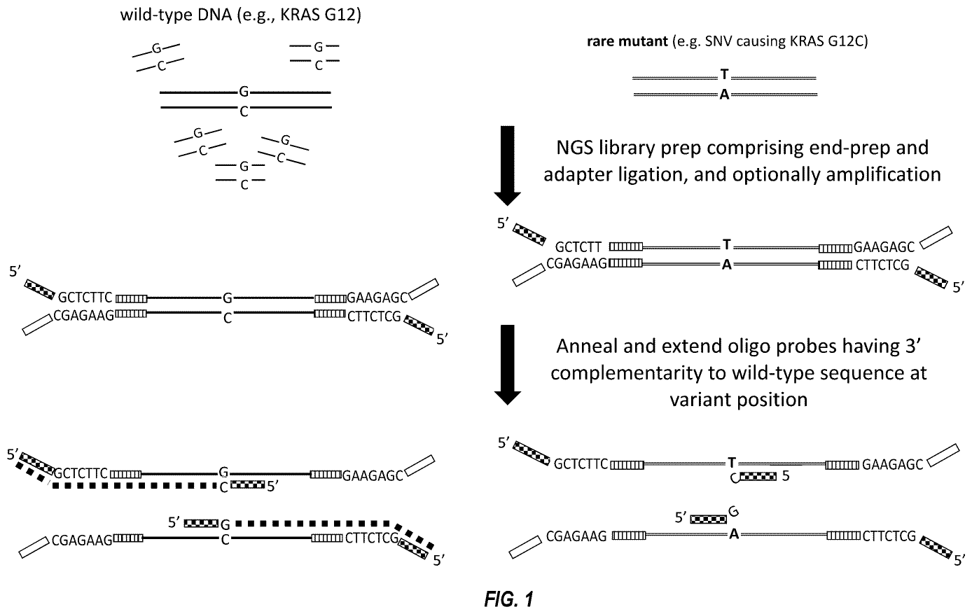

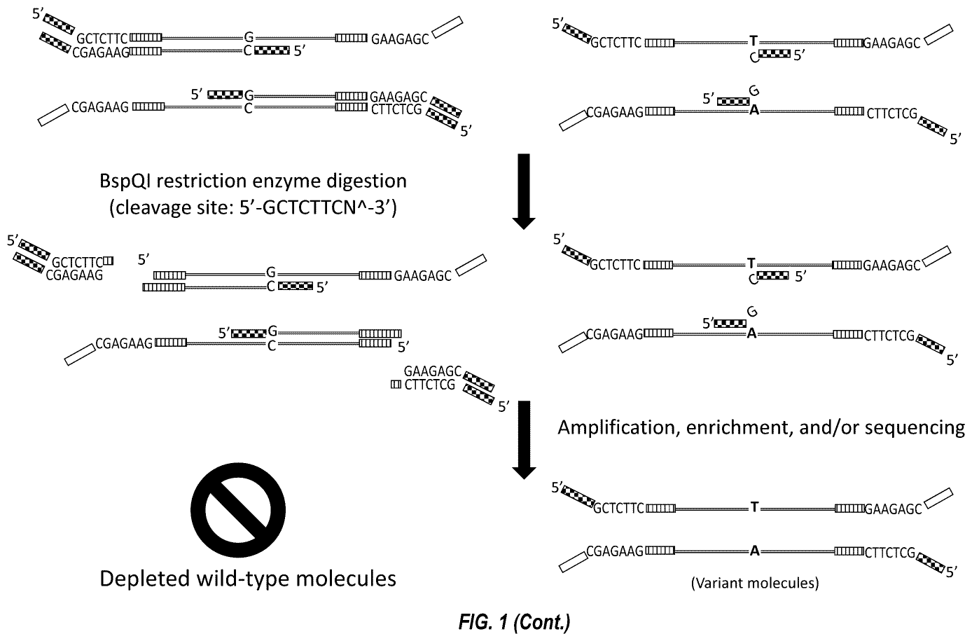

In some embodiments, the procedure that affects a first nucleobase in the DNA differently from a second nucleobase in the DNA comprises Tet-assisted conversion with a substituted borane reducing agent, optionally wherein the substituted borane reducing agent is 2-picoline borane, borane pyridine, tert-butylamine borane, or ammonia borane. In Tet-assisted pic-borane conversion with a substituted borane reducing agent conversion, a TET protein is used to convert mC and hmC to caC, without affecting unmodified C. caC, and fC if present, are then converted to dihydrouracil (DHU) by treatment with 2-picoline borane (pic-borane) or another substituted borane reducing agent such as borane pyridine, tert-butylamine borane, or ammonia borane, also without affecting unmodified C. See, e.g., Liu et al., Nature Biotechnology 2019; 37:424-429 (e.g., at Supplementary FIG. 1 and Supplementary Note 7). Thus, when this type of conversion is used, the first nucleobase comprises one or more of 5mC, 5fC, 5caC, or 5hmC, and the second nucleobase comprises unmodified cytosine. DHU is read as a T in sequencing. Thus, when this type of conversion is used, the first nucleobase comprises one or more of mC, fC, caC, or hmC, and the second nucleobase comprises unmodified cytosine. Sequencing of the converted DNA identifies positions that are read as cytosine as being unmodified C positions. Meanwhile, positions that are read as T are identified as being T, mC, fC, caC, or hmC. Performing TAP conversion, such as on a DNA sample as described herein, thus facilitates identifying positions containing unmodified C using the sequence reads obtained from the sample. This procedure encompasses Tet-assisted pyridine borane sequencing (TAPS), described in further detail in Liu et al. 2019, supra.

Alternatively, protection of hmC (e.g., using PGT or 5-hydroxymethylcytosine carbamoyltransferase) can be combined with Tet-assisted conversion with a substituted borane reducing agent, e.g. as described above. In this method (TAPS-β), 5hmC can be protected from conversion, for example through glucosylation using β-glucosyl transferase (OGT), forming 5-glucosylhydroxymethylcytosine (5ghmC), or through carbamoylation using 5-hydroxymethylcytosine carbamoyltransferase, forming 5cmC. This is described in Yu et al., Cell 2012; 149: 1368-80. Treatment with a TET protein, such as mTet1 or a TET2 comprising a T1372S mutation, then converts mC to caC but does not convert C, 5ghmC, or 5cmC. 5caC is then converted to DHU by treatment with pic-borane or another substituted borane reducing agent such as borane pyridine, tert-butylamine borane, or ammonia borane, also without affecting ghmC, 5cmC, or unmodified C. Thus, when Tet-assisted conversion with a substituted borane reducing agent is used, the first nucleobase comprises mC, and the second nucleobase comprises one or more of unmodified cytosine or hmC, such as unmodified cytosine and optionally hmC, fC, and/or caC. Sequencing of the converted DNA identifies positions that are read as cytosine as being either hmC or unmodified C positions. Meanwhile, positions that are read as T are identified as being T, fC, caC, or mC. Performing TAPSβ conversion, such as on a DNA sample as described herein, thus facilitates distinguishing positions containing unmodified C or hmC on the one hand from positions containing mC using the sequence reads obtained from the sample. For an exemplary description of this type of conversion, see, e.g., Liu et al., Nature Biotechnology 2019; 37:424-429. 5-hydroxymethylcytosine carbamoyltransferase is described in Yang et al., Bio-protocol, 2023; 12(17): e4496.

In some embodiments, the procedure that affects a first nucleobase in the DNA differently from a second nucleobase in the DNA comprises chemical-assisted conversion with a substituted borane reducing agent, optionally wherein the substituted borane reducing agent is 2-picoline borane, borane pyridine, tert-butylamine borane, or ammonia borane. In chemical-assisted conversion with a substituted borane reducing agent, an oxidizing agent such as potassium perruthenate (KRuO₄) (also suitable for use in ox-BS conversion) is used to specifically oxidize hmC to fC. Treatment with pic-borane or another substituted borane reducing agent such as borane pyridine, tert-butylamine borane, or ammonia borane converts fC and caC to DHU but does not affect mC or unmodified C. Thus, when this type of conversion is used, the first nucleobase comprises one or more of hmC, fC, and caC, and the second nucleobase comprises one or more of unmodified cytosine or mC, such as unmodified cytosine and optionally mC. Sequencing of the converted DNA identifies positions that are read as cytosine as being either mC or unmodified C positions. Meanwhile, positions that are read as T are identified as being T, fC, caC, or hmC. Performing this type of conversion, such as on a DNA sample as described herein, thus facilitates distinguishing positions containing unmodified C or mC on the one hand from positions containing hmC using the sequence reads obtained from the sample. For an exemplary description of this type of conversion, see, e.g., Liu et al., Nature Biotechnology 2019; 37:424-429.

In some embodiments, the procedure that affects a first nucleobase in the DNA differently from a second nucleobase in the DNA comprises APOBEC-coupled epigenetic (ACE) conversion. In ACE conversion, an AID/APOBEC family DNA deaminase enzyme such as APOBEC3A (A3A) is used to deaminate unmodified cytosine and mC without deaminating hmC, fC, or caC. Thus, when ACE conversion is used, the first nucleobase comprises unmodified C and/or mC (e.g., unmodified C and optionally mC), and the second nucleobase comprises hmC. Sequencing of ACE-converted DNA identifies positions that are read as cytosine as being hmC, fC, or caC positions. Meanwhile, positions that are read as T are identified as being T, unmodified C, or mC. Performing ACE conversion on a DNA sample as described herein thus facilitates distinguishing positions containing hmC from positions containing mC or unmodified C using the sequence reads obtained from the sample. For an exemplary description of ACE conversion, see, e.g., Schutsky et al., Nature Biotechnology 2018; 36: 1083-1090.

In some embodiments, the procedure that affects a first nucleobase in the DNA differently from a second nucleobase in the DNA comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-PGT or 5-hydroxymethylcytosine carbamoyltransferase (described in Yang et al., Bio-protocol, 2023; 12(17): e4496) can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA comprises enzymatic conversion of the first nucleobase using a non-specific, modification-sensitive double-stranded DNA deaminase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at https://www.biorxiv.org/content/10.1101/2023.06.29.547047v1. SEM-Seq employs a non-specific, modification-sensitive double-stranded DNA deaminase (MsddA) in a nondestructive single-enzyme 5-methylctyosine sequencing (SEM-seq) method that deaminates unmodified cytosines. Accordingly, SEM-seq does not require the TET2 and T4-βGT or 5-hydroxymethylcytosine carbamoyltransferase protection and denaturing steps that are of use, e.g., in APOEC3A-based protocols. Additionally, MsddA does not deaminate 5-formylated cytosines (5fC) or 5-carboxylated cytosines (5caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained. In some embodiments, the procedure that affects a first nucleobase in the DNA differently from a second nucleobase in the DNA comprises enzymatic conversion of the first nucleobase using MsddA.

In some embodiments, the procedure that affects a first nucleobase in the DNA differently from a second nucleobase in the DNA of the first subsample converts a modified nucleoside. In some embodiments, the conversion procedure which converts a modified nucleosides comprises enzymatic conversion, such as DM-seq, for example, as described in WO2023/288222A1. In DM-seq, unmodified cytosines in the DNA are enzymatically protected from a subsequent deamination step wherein 5mC in 5mCpG is converted to T. The enzymatically protected unmodified (e.g., unmethylated) cytosines are not converted and are read as “C” during sequencing. Cytosines that are read as thymines (in a CpG context) are identified as methylated cytosines in the DNA. Thus, when this type of conversion is used, the first nucleobase comprises unmodified (such as unmethylated) cytosine, and the second nucleobase comprises modified (such as methylated) cytosine. Sequencing of the converted DNA identifies positions that are read as cytosine as being unmodified C positions. Meanwhile, positions that are read as T are identified as being T or 5mC. Performing DM-seq conversion thus facilitates identifying positions containing 5mC using the sequence reads obtained.

Exemplary cytosine deaminases for use herein include APOBEC enzymes, for example, APOBEC3A. Generally, AID/APOBEC family DNA deaminase enzymes such as APOBEC3A (A3A) are used to deaminate (unprotected) unmodified cytosine and 5mC. For an exemplary description of APOBEC conversion, see, e.g., Schutsky et al., Nature Biotechnology 2018; 36: 1083-1090.

The enzymatic protection of unmodified cytosines in the DNA comprises addition of a protective group to the unmodified cytosines. Such protective groups can comprise an alkyl group, an alkyne group, a carboxyl group, a carboxyalkyl group, an amino group, a hydroxymethyl group, a glucosyl group, a glucosylhydroxymethyl group, an isopropyl group, or a dye. For example, DNA can be treated with a methyltransferase, such as a CpG-specific methyltransferase, which adds the protective group to unmodified cytosines. The term methyltransferase is used broadly herein to refer to enzymes capable of transferring a methyl or substituted methyl (e.g., carboxymethyl) to a substrate (e.g., a cytosine in a nucleic acid). In some embodiments, the DNA is contacted with a CpG-specific DNA methyltransferase (MTase), such as a CpG-specific carboxymethyltransferase (CxMTase), and a substituted methyl donor, such as a carboxymethyl donor (e.g., carboxymethyl-S-adenosyl-L-methionine). See, e.g., WO2021/236778A2. In particular embodiments, the CxMTase can facilitate the addition of a protective carboxymethyl group to an unmethylated cytosine. In some embodiments, the unmethylated cytosine is unmodified cytosine. The carboxymethyl group can prevent deamination of the cytosine during a deamination step (such as a deamination step using an APOBEC enzyme, such as A3A). Substituted methyl or carboxymethyl donors useful in the disclosed methods include but are not limited to, S-adenosyl-L-methionine (SAM) analogs, optionally wherein the SAM analog is carboxy-S-adenosyl-L-methionine (CxSAM). SAM analogs are described, for example, in WO2022/197593A1. The MTase may be, for example, a CpG methyltransferase from Spiroplasma sp. strain MQ1 (M.SssI), DNA-methyltransferase 1 (DNMT1), DNA-methyltransferase 3 alpha (DNMT3A), DNA-methyltransferase 3 beta (DNMT3B), or DNA adenine methyltransferase (Dam). The CxMTase may be a CpG methyltransferase from Mycoplasma penetrans (M.MpeI). In a particular embodiment, the methyltransferase enzyme is a variant of M.MpeI having SEQ ID NO: 1 or SEQ ID NO: 2, or a sequence at least 90%, at least 92%, at least 94%, at least 96%, at least 97%, at least 98%, or at least 99% identical thereto, optionally wherein the amino acid corresponding to position 374 is R or K.

In one embodiment, the methyltransferase enzyme is a variant of M.MpeI having an N374R substitution or an N374K substitution. The methyltransferase of SEQ ID NO: 1 or SEQ ID NO: 2 can further comprise one or more amino acid substitutions selected from a) substitution of one or both residues T300 and E305 with S, A, G, Q, D, or N; b) substitution of one or more residues A323, N306, and Y299 with a positively charged amino acid selected from K, R or H; and/or c) substitution of S323 with A, G, K, R or H, which may enhance the activity of the enzyme.

Optionally, the conversion procedure further includes enzymatic protection of 5hmCs, such as by glucosylation of the 5hmCs (e.g., using βGT) or by carbamoylation of the 5hmCs (e.g., using 5-hydroxymethylcytosine carbamoyltransferase), in the DNA prior to the deamination of unprotected modified cytosines. In this method, 5hmC can be protected from conversion, for example through glucosylation using β-glucosyl transferase (βGT), forming (5-glucosylhydroxymethylcytosine) 5ghmC, or through carbamoylation using 5-hydroxymethylcytosine carbamoyltransferase, forming 5cmC. This is described, for example, in Yu et al., Cell 2012; 149: 1368-80, and in Yang et al., Bio-protocol, 2023; 12(17): e4496. Glucosylation or carbamoylation of 5hmC can reduce or eliminate deamination of 5hmC by a deaminase such as APOBEC3A. Treatment with an MTase or CxMTase then adds a protecting group to unmodified (unmethylated) cytosines in the DNA. 5mC (but not protected, unmodified cytosine and not 5ghmC or 5cmC) is then deaminated (converted to T in the case of 5mC) by treatment with a deaminase, for example, an APOBEC enzyme (such as APOBEC3A). Sequencing of the converted DNA identifies positions that are read as cytosine as being either 5hmC or unmodified C positions. Meanwhile, positions that are read as T are identified as being T or 5mC. Performing DM-seq conversion with glucosylation of 5hmC on a sample as described herein thus facilitates distinguishing positions containing unmodified C or 5hmC on the one hand from positions containing 5mC using the sequence reads obtained.

Also provided herein are methods in which alternative base conversion schemes are used. For example, unmethylated cytosines can be left intact while methylated cytosines and hydroxymethylcytosines are converted to a base read as a thymine (e.g., uracil, thymine, or dihydrouracil).

In some embodiments, methylating a cytosine in at least one first complementary strand or second complementary strand comprises contacting the cytosine with a methyltransferase such as DNMT1 or DNMT5. In such embodiments, the step of oxidizing a 5-hydroxymethylated cytosine to 5-formylcytosine (such as by contacting the 5-hydroxymethyl cytosine in a first strand and a second strand with KRuO₄) can be optional.

In some embodiments, converting the modified cytosine in at least one first or second strand to a thymine or a base read as thymine comprises oxidizing a hydroxymethyl cytosine, e.g., the hydroxymethyl cytosine is oxidized to formylcytosine. In some embodiments, oxidizing the hydroxymethyl cytosine to formylcytosine comprises contacting the hydroxymethyl cytosine with a ruthenate, such as potassium ruthenate (KRuO₄).

In some embodiments, the modified cytosine is converted to thymine, uracil, or dihydrouracil. In any such embodiments, amplification methods may comprise uracil- and/or dihydrouracil-tolerant amplification methods, such as PCR using a uracil- and/or dihydrouracil-tolerant DNA polymerase.

In some embodiments, the method comprises converting a formylcytosine and/or a methylcytosine to carboxylcytosine as part of converting the modified cytosine in at least one first or second strand to a thymine or a base read as thymine. For example, converting the formylcytosine and/or the methylcytosine to carboxylcytosine can comprise contacting the formylcytosine and/or the methylcytosine with a TET enzyme, such as TET1, TET2, TET3, or a TET2 comprising a T1372S mutation. In some embodiments, the method comprises reducing the carboxylcytosine as part of converting the modified cytosine in at least one first or second strand to a thymine or a base read as thymine, and/or the carboxylcytosine is reduced to dihydrouracil. In some embodiments, reducing the carboxylcytosine comprises contacting the carboxylcytosine with a borane or borohydride reducing agent.

In some embodiments, the borane or borohydride reducing agent comprises pyridine borane, 2-picoline borane, borane, tert-butylamine borane, ammonia borane, sodium borohydride, sodium cyanoborohydride (NaBH₃CN), lithium borohydride (LiBH₄), ethylenediamine borane, dimethylamine borane, sodium triacetoxyborohydride, morpholine borane, 4-methylmorpholine borane, trimethylamine borane, dicyclohexylamine borane, or a salt thereof. In other embodiments, the reducing agent comprises lithium aluminum hydride, sodium amalgam, amalgam, sulfur dioxide, dithionate, thiosulfate, iodide, hydrogen peroxide, hydrazine, diisobutylaluminum hydride, oxalic acid, carbon monoxide, cyanide, ascorbic acid, formic acid, dithiothreitol, beta-mercaptoethanol, or any combination thereof.

Various TET enzymes may be used in the disclosed methods as appropriate. In some embodiments, the one or more TET enzymes comprise TETv. TETv is described in U.S. Pat. No. 10,260,088 and its sequence is SEQ ID NO: 1 therein (SEQ ID NO: 3 in the present application). In some embodiments, the one or more TET enzymes comprise TETcd. TETcd is described in U.S. Pat. No. 10,260,088 and its sequence is SEQ ID NO: 3 therein (SEQ ID NO: 4 in the present application). In some embodiments, the one or more TET enzymes comprise TET1. In some embodiments, the one or more TET enzymes comprise TET2. TET2 may be expressed and used as a fragment comprising TET2 residues 1129-1480 joined to TET2 residues 1844-1936 by a linker (SEQ ID NO: 5 of the present application) as described, e.g., in U.S. Pat. No. 10,961,525. In some embodiments, the one or more TET enzymes comprise TET1 and TET2. In some embodiments, the one or more TET enzymes comprise a V1900 TET mutant, such as a V1900A, V1900C, V1900G, V1900I, or V1900P TET mutant. In some embodiments, the one or more TET enzymes comprise a V1900 TET2 mutant, such as a V1900A, V1900C, V1900G, V19001, or V1900P TET2 mutant. Examples of V1900A, V1900C, V1900G, V19001, and V1900P TET2 mutants are provided as SEQ ID NOs: 6-10. In some embodiments, the V1900 TET mutant has at least 80%, 85%, 90%, 95%, 96%, 97%, 98%, or 99% sequence identity to SEQ ID NO: 6, 7, 8, 9, or 10. Position 1900 of the wild-type TET2 sequence corresponds to position 438 in each of SEQ ID NOs: 5-10. It can be beneficial to use a TET enzyme that maximizes formation of 5-carboxylcytosine (5-caC) relative to less oxidized modified cytosines, particularly 5-formylcytosine, because 5-caC is not a substrate for enzymatic deamination, e.g., by APOBEC enzymes such as APOBEC3A. Maximizing formation of 5-caC thus reduces the risk of false calls in which a base is identified as unmethylated because it underwent deamination even though it was methylated (or hydroxymethylated) in the original sample. Accordingly, in some embodiments, the TET enzyme comprises a mutation that increases formation of 5-caC. Exemplary mutations are set forth above. “A mutation that increases formation of 5-caC” means that the TET enzyme having the mutation produces more 5-caC than a TET enzyme that lacks the mutation but is otherwise identical. 5-caC production can be measured as described, e.g., in Liu et al., Nat Chem Biol 13:181-187 (2017) (see Online Methods section, TET reactions in vitro subsection, “driving” conditions). Any variants and/or mutants described in Liu et al. (2017) can be used in the disclosed methods as appropriate.

In some embodiments, the one or more TET enzymes comprise a TET2 enzyme comprising a T1372S mutation, such as TET2-CS-T1372S and TET2-CD-T1372S. Examples of TET2-CS-T1372S and TET2-CD-T1372S are provided as SEQ ID NOs: 11 and 12. A TET2 comprising a T1372S mutation is described in U.S. Pat. No. 10,961,525 and may be expressed and used as a fragment comprising TET2 residues 1129-1480 joined to TET2 residues 1844-1936 by a linker. Position 1372 of TET2 corresponds to position 258 of SEQ ID NO: 21 (wild type TET2 catalytic domain) of U.S. Pat. No. 10,961,525. Thus, the sequence of a T1372S TET2 catalytic domain may be obtained by changing the threonine at position 258 of SEQ ID NO: 21 of U.S. Pat. No. 10,961,525 to serine. TET2 comprising a T1372S mutation is also described in Liu et al., Nat Chem Biol. 2017 February; 13(2): 181-187. As demonstrated in Liu et al., TET2 comprising a T1372S mutation can more efficiently oxidize 5mC to produce 5-carboxylcytosine (5caC) than other versions of TET2 such as TET2 lacking a T1372S mutation. In some embodiments, the TET2 enzyme comprises SEQ ID NO: 14 or optionally a variant of SEQ ID NO: 14 in which at least 5, 6, 7, or 8 positions match SEQ ID NO: 14 including position 5 of SEQ ID NO: 14. In some embodiments, the TET2 enzyme is a human TET2 enzyme comprising a T1372S mutation. In some embodiments, the TET2 enzyme comprises the sequence of SEQ ID NO: 11. In some embodiments, the TET2 enzyme comprises a sequence having at least 80%, 85%, 90%, 95%, 96%, 97%, 98%, or 99% identity to SEQ ID NO: 11. In some embodiments, the TET2 enzyme comprises a sequence having at least 80%, 85%, 90%, 95%, 96%, 97%, 98%, or 99% identity to SEQ ID NO: 12. In some embodiments, the TET2 enzyme comprises the sequence of SEQ ID NO: 12. The sequences of SEQ ID NOs: 11 and 12 are shown in the Table of Sequences herein.

Provided herein is a method comprising contacting DNA contacting DNA with a TET2 enzyme comprising a T1372S mutation to oxidize 5-methylcytosine (5mC) and/or 5-hydroxymethylcytosine (5hmC) present in the DNA to 5-carboxycytosine (5caC), subsequently contacting at least a portion of the DNA with a substituted borane reducing agent, thereby converting 5-caC in the DNA to dihydrouracil (DHU), thereby producing treated DNA, and sequencing at least a portion of the treated DNA.

In some embodiments, the procedure that affects a first nucleobase in the DNA differently from a second nucleobase in the DNA comprises separating DNA originally comprising the first nucleobase from DNA not originally comprising the first nucleobase. In some such embodiments, the first nucleobase is hmC. DNA originally comprising the first nucleobase may be separated from other DNA using a labeling procedure comprising biotinylating positions that originally comprised the first nucleobase. In some embodiments, the first nucleobase is first derivatized with an azide-containing moiety, such as a glucosyl-azide containing moiety. The azide-containing moiety then may serve as a reagent for attaching biotin, e.g., through Huisgen cycloaddition chemistry. Then, the DNA originally comprising the first nucleobase, now biotinylated, can be separated from DNA not originally comprising the first nucleobase using a biotin-binding agent, such as avidin, neutravidin (deglycosylated avidin with an isoelectric point of about 6.3), or streptavidin. An example of a procedure for separating DNA originally comprising the first nucleobase from DNA not originally comprising the first nucleobase is hmC-seal, which labels hmC to form β-6-azide-glucosyl-5-hydroxymethylcytosine and then attaches a biotin moiety through Huisgen cycloaddition, followed by separation of the biotinylated DNA from other DNA using a biotin-binding agent. For an exemplary description of hmC-seal, see, e.g., Han et al., Mol. Cell 2016; 63: 711-719. This approach is useful for identifying fragments that include one or more hmC nucleobases.

In some embodiments, following such a separation, the method further comprises differentially tagging each of the DNA originally comprising the first nucleobase, the DNA not originally comprising the first nucleobase. The method may further comprise pooling the DNA originally comprising the first nucleobase and the DNA not originally comprising the first nucleobase following differential tagging. The DNA originally comprising the first nucleobase and the DNA not originally comprising the first nucleobase may then be used in downstream analyses. For example, the pooled DNA originally comprising the first nucleobase and the DNA not originally comprising the first nucleobase may be sequenced in the same sequencing cell (such as after being subjected to further treatments, such as those described herein) while retaining the ability to resolve whether a given read came from a molecule of DNA originally comprising the first nucleobase or DNA not originally comprising the first nucleobase using the differential tags.

In some embodiments, the first nucleobase is a modified or unmodified adenine, and the second nucleobase is a modified or unmodified adenine. In some embodiments, the modified adenine is N⁶-methyladenine (mA). In some embodiments, the modified adenine is one or more of N⁶-methyladenine (mA), N⁶-hydroxymethyladenine (hmA), or N⁶-formyladenine (fA).

Techniques comprising partitioning based on methylation status or methylated DNA immunoprecipitation (MeDIP) can be used to separate DNA containing modified bases such as mC, mA, caC (which may be generated by oxidation of mC or hmC with Tet2, e.g., before enzymatic conversion of unmodified C to U, e.g., using a deaminase such as APOBEC3A), or dihydrouracil from other DNA. See, e.g., Kumar et al., Frontiers Genet. 2018; 9: 640; Greer et al., Cell 2015; 161: 868-878. An antibody specific for mA is described in Sun et al., Bioessays 2015; 37:1155-62. Antibodies for various modified nucleobases, such as mC, caC, and forms of thymine/uracil including dihydrouracil or halogenated forms such as 5-bromouracil, are commercially available. Various modified bases can also be detected based on alterations in their base pairing specificity. For example, hypoxanthine is a modified form of adenine that can result from deamination and is read in sequencing as a G. See, e.g., U.S. Pat. No. 8,486,630; Brown, Genomes, 2^{nd} Ed., John Wiley & Sons, Inc., New York, N.Y., 2002, chapter 14, “Mutation, Repair, and Recombination.”

H. Partitioning the Sample into a Plurality of Subsamples

In some instances, a heterogeneous nucleic acid sample is partitioned into a plurality (two or more) partitions (sub-samples). In some embodiments, each partition is differentially tagged. Tagged partitions can then be pooled together for collective sample prep and/or sequencing. The partitioning-tagging-pooling steps can occur more than once, with each round of partitioning occurring based on a different characteristics, and tagged using differential tags that are distinguished from other partitions and partitioning means.

In some embodiments, the partitioning is performed prior to a step of sequencing and/or (a) prior to or after a step of selectively depleting the target nucleic acid comprising the wild-type sequence, the target nucleic acid comprising the converted nucleotide, or the target nucleic acid that does not comprise the converted nucleotide; (b) prior to or after a step of amplifying the selectively digested population of target nucleic acids; (c) prior to or after a step of subjecting the population of target nucleic acids to a procedure that affects a first nucleobase of the DNA differently from a second nucleobase of the DNA; and/or (d) prior to or after a step of enriching for one or more sets of target regions of DNA.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some cases, different procedures are applied to different partitions to determine different characteristics of the initial sample. The nucleic acid, e.g., DNA (such as cDNA), of at least one partition is subjected to a conversion procedure according to the methods of the disclosure described herein. In some embodiments at least one partition is not subjected to the conversion procedure. Corresponding sequences from the converted and non-converted partitions can be compared to identify single nucleotides that have undergone conversion and therefore identify corresponding modified nucleosides in the initial sample.

For methods that involve a partitioning step, a partition tag (which distinguishes molecules in one partition from those in a different partition) may be included in the adapters or may be added to the sample molecules.

In some embodiments, two or more partitions, e.g., each partition, is/are differentially tagged. Tags can be used to label the individual polynucleotide population partitions so as to correlate the tag (or tags) with a specific partition. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108: 9530-9535 (2011), Kou et al., PLoS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics, the final tagged molecules in the library may no longer possess that characteristic. For example, while single stranded DNA molecules may be partitioned and tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to a molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a whole DNA population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in genomic region while lower coverage can correlate with lower nucleosome occupancy or a nucleosome depleted region (NDR).

Disclosed methods herein comprise analyzing DNA in a sample. In some embodiments described herein, the disclosed methods comprise partitioning DNA. In such methods, different forms of DNA (e.g., hypermethylated and hypomethylated DNA) can be physically partitioned based on one or more characteristics of the DNA. This approach can be used to determine, for example, whether certain sequences are hypermethylated or hypomethylated. In some embodiments, a first subsample or aliquot of a sample is subjected to steps for making capture probes as described elsewhere herein and a second subsample or aliquot of a sample is subjected to partitioning. In some embodiments, a sample or subsample or aliquot thereof is subjected to partitioning and differential tagging, followed by a capture step using capture probes for rearranged sequences and optionally additional capture probes, e.g., for sequence-variable and/or epigenetic target regions.

Methylation profiling can involve determining methylation patterns across different regions of the genome. For example, after partitioning molecules based on extent of methylation (e.g., relative number of methylated nucleobases per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

Partitioning nucleic acid molecules in a sample can increase a rare signal, e.g., by enriching rare nucleic acid molecules that are more prevalent in one partition of the sample. For example, a genetic variation present in hypermethylated DNA but less (or not) present in hypomethylated DNA can be more easily detected by partitioning a sample into hypermethylated and hypomethylated nucleic acid molecules. By analyzing multiple partitions of a sample, a multi-dimensional analysis of a single molecule can be performed and hence, greater sensitivity can be achieved. Partitioning may include physically partitioning nucleic acid molecules into partitions or subsamples based on the presence or absence of one or more methylated nucleobases. A sample may be partitioned into partitions or subsamples based on a characteristic that is indicative of differential gene expression or a disease state. A sample may be partitioned based on a characteristic, or combination thereof that provides a difference in signal between a normal and diseased state during analysis of nucleic acids, e.g., cDNA generated from RNA from a sample, cell free DNA (cfDNA), non-cfDNA, tumor DNA, circulating tumor DNA (ctDNA) and cell free nucleic acids (cfNA).

In some embodiments, hypermethylation and/or hypomethylation variable epigenetic target regions are analyzed to determine whether they show differential methylation characteristic of tumor cells or cells of a type that does not normally contribute to the DNA sample being analyzed (such as cfDNA), and/or particular immune cell types.

In some instances, heterogeneous DNA in a sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristic (examples provided herein), and tagged using differential tags that are distinguished from other partitions and partitioning means. In other instances, the differentially tagged partitions are separately sequenced.

In some embodiments, sequence reads from differentially tagged and pooled DNA are obtained and analyzed in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

The agents used to partition populations of nucleic acids within a sample can be affinity agents, such as antibodies with the desired specificity, natural binding partners or variants thereof (Bock et al., Nat Biotech 28: 1106-1114 (2010); Song et al., Nat Biotech 29: 68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Alternative partitioning agents include methyl binding domain (MBDs) and methyl binding proteins (MBPs) as described herein, including proteins such as MeCP2.

Additional, non-limiting examples of partitioning agents are histone binding proteins which can separate nucleic acids bound to histones from free or unbound nucleic acids. Examples of histone binding proteins that can be used in the methods disclosed herein include RBBP4, RbAp48 and SANT domain peptides.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., MethylMinder Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

Analyzing DNA may comprise detecting or quantifying DNA of interest. Analyzing DNA can comprise detecting genetic variants and/or epigenetic features (e.g., DNA methylation and/or DNA fragmentation).

In some embodiments, methylation levels can be determined using partitioning, modification-sensitive conversion such as DM-seq, direct detection during sequencing, methylation-sensitive restriction enzyme digestion, methylation-dependent restriction enzyme digestion, or any other suitable approach. For example, different forms of DNA (e.g., hypermethylated and hypomethylated DNA) can be physically partitioned based on one or more characteristics of the DNA. For example, a methylated DNA binding protein (e.g., an MBD such as MBD2, MBD4, or MeCP2) or an antibody specific for 5-methylcytosine (as in MeDIP) can be used to partition the DNA. This approach can be used to determine, for example, whether certain sequences are hypermethylated or hypomethylated. In some embodiments, DNA fragmentation pattern can be determined based on endpoints and/or centerpoints of DNA molecules, such as cfDNA molecules.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e., in solution). The nucleic acids in the bound phase can be eluted before subsequent processing.

When using MeDIP or MethylMiner®Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the partitioning comprises contacting the DNA with a methylation sensitive restriction enzyme (MSRE) and/or a methylation dependent restriction enzyme (MDRE). Following the treatment of the DNA with a MSRE or a MDRE, the DNA may be partitioned based on size to generate hypermethylated (longest DNA molecules following MSRE treatment and shortest DNA fragments following MDRE treatment), intermediate (intermediate length DNA molecules following MSRE or MDRE treatment), and hypomethylated (shortest DNA molecules following MSRE treatment and longest DNA fragments following MDRE treatment) subsamples.

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed above.

Examples of agents that recognize a modified nucleobase contemplated herein include, but are not limited to:
(a) MeCP2 is a protein that preferentially binds to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formylcytosine over unmodified cytosine (Iurlaro et al., Genome Biol. 14: R119 (2013)).
(d) Antibodies specific to one or more methylated or modified nucleobases or conversion products thereof, such as 5mC, 5caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads.

In some embodiments, the partitions of DNA are desalted and concentrated in preparation for enzymatic steps of library preparation.

Sequences that comprise aberrantly high copy numbers may tend to be hypermethylated. Accordingly, in some embodiments, the DNA contacted with capture probes specific for members of an epigenetic target region set comprising a plurality of target regions that are both type-specific differentially methylated regions and copy number variants comprises at least a portion of a hypermethylated partition. The DNA from or comprising at least a portion of the hypermethylated partition may or may not be combined with DNA from or comprising at least a portion of one or more other partitions, such as an intermediate partition or a hypomethylated partition.

In some embodiments, methylation is detected using a conversion procedure. Conversion procedures include any technique that differentially alters a first nucleobase but not a second nucleobase in a modification-dependent manner, e.g., being methylated (or hydroxymethylated, or formylated, or carboxylated, etc.) versus unmodified, and/or being modified in one way versus another way (e.g., methylated versus hydroxymethylated). Examples of such conversion procedures include conversion with a deaminase, which converts cytosine to uracil whereas protected modified cytosines (e.g., 5ghmC, 5caC) are not converted. It can be beneficial to include adapters comprising deamination-resistant modified cytosines in DNA that will be subject to deamination, so that the adapters are not affected by deamination. For an exemplary description of such adapters and deamination-resistant modified cytosines, see WO2023/288222A1.

In some embodiments, methylation detection comprises using a methylation-sensitive restriction enzyme (MSRE). For example, a sample, subsample, or portion of a sample can be subjected to digestion with one or more MSREs to cleave unmethylated sequences. Exemplary MSREs include AatII, AccII, AciI, Aor13HI, Aor15HI, BspT104I, BssHII, BstUI, Cfr10I, ClaI, CpoI, Eco52I, HaeII, HapII, HhaI, Hin6I, HpaII, HpyCH4IV, MluI, MspI, NaeI, NotI, NruI, NsbI, PmaCI, Psp14061, PvuI, SacII, SalI, SmaI, and SnaBI. In some embodiments, at least two methylation-sensitive nucleases are used. In some embodiments, at least three methylation-sensitive nucleases are used. In some embodiments, the methylation-sensitive nucleases comprise BstUI and HpaII. In some embodiments, the two methylation-sensitive nucleases comprise HhaI and AccII. In some embodiments, the methylation-sensitive nucleases comprise BstUI, HpaII and Hin6I. In some embodiments, the portion of the sample that is contacted with one or more MSREs comprises hypermethylated DNA, or is or comprises a hypermethylated DNA partition, which may be obtained as described elsewhere herein.

In some embodiments, DNA fragmentation is detected by determining the endpoints and/or midpoints of sequenced fragments of DNA (e.g., cfDNA). For example, differences in fragmentation patterns may occur depending on whether the fragments originated from a tumor or from healthy cells. To detect tumor-cell derived DNA of cfDNA based on fragmentation, the presence or absence of an increased level of abnormal fragments can be determined at regions with copy-number amplifications, (e.g., proportional to the degree of amplification), e.g., where the increase and abnormality are relative to control or healthy samples.

In some embodiments, a sample or subsample (e.g., a first, second, or third subsample prepared by partitioning a sample as described herein, such as on the basis of a level of a cytosine modification, such as methylation, e.g., 5-methylation, such as of cytosine) is contacted with a methylation-dependent nuclease or methylation-sensitive nuclease. Unless otherwise indicated, where partitioning is performed on the basis of a cytosine modification, the first subsample is the subsample with a higher level of the modification; the second subsample is the subsample with a lower level of the modification; and, when present, the third subsample has a level of the modification intermediate between the first and second subsamples.

As discussed above, partitioning procedures may result in imperfect sorting of DNA molecules among the subsamples. The choice of a methylation-dependent nuclease or methylation-sensitive nuclease can be made so as to degrade nonspecifically partitioned DNA. For example, the second subsample can be contacted with a methylation-dependent nuclease, such as a methylation-dependent restriction enzyme. This can degrade nonspecifically partitioned DNA in the second subsample (e.g., methylated DNA) to produce a treated second subsample. Alternatively or in addition, the first subsample can be contacted with a methylation-sensitive endonuclease, such as a methylation-sensitive restriction enzyme, thereby degrading nonspecifically partitioned DNA in the first subsample to produce a treated first subsample. Degradation of nonspecifically partitioned DNA in either or both of the first or second subsamples is proposed as an improvement to the performance of methods that rely on accurate partitioning of DNA on the basis of a cytosine modification, e.g., to detect the presence of aberrantly modified DNA in a sample, to determine the tissue of origin of DNA, and/or to determine whether a subject has cancer. For example, such degradation may provide improved sensitivity and/or simplify downstream analyses. In general, where nonspecifically partitioned DNA would be hypermethylated, such as in a hypomethylated partition, a methylation-dependent nuclease, such as a methylation-dependent restriction enzyme, should be used. Conversely, where nonspecifically partitioned DNA would be hypomethylated, such as in a hypermethylated partition, a methylation-sensitive nuclease, such as a methylation-sensitive restriction enzyme, should be used. Methylation-dependent nucleases, such as methylation-dependent restriction enzymes, preferentially cut methylated DNA relative to unmethylated DNA, while methylation-sensitive nucleases, such as methylation-sensitive restriction enzymes, preferentially cut unmethylated DNA relative to methylated DNA.

In contacting a subsample with a nuclease, one or more nucleases can be used. In some embodiments, a subsample is contacted with a plurality of nucleases. The subsample may be contacted with the nucleases sequentially or simultaneously. Simultaneous use of nucleases may be advantageous when the nucleases are active under similar conditions (e.g., buffer composition) to avoid unnecessary sample manipulation. Contacting the second subsample with more than one methylation-dependent restriction enzyme can more completely degrade nonspecifically partitioned hypermethylated DNA. Similarly, contacting the first subsample with more than one methylation-sensitive restriction enzyme can more completely degrade nonspecifically partitioned hypomethylated and/or unmethylated DNA.

In some embodiments, a methylation-dependent nuclease comprises one or more of MspJI, LpnPI, FspEI, or McrBC. In some embodiments, at least two methylation-dependent nucleases are used. In some embodiments, at least three methylation-dependent nucleases are used. In some embodiments, the methylation-dependent nuclease comprises FspEI. In some embodiments, the methylation-dependent nuclease comprises FspEI and MspJI, e.g., used sequentially.

In some embodiments, a methylation-sensitive nuclease comprises one or more of AatII, AccII, AciI, Aor13HI, Aor15HI, BspT104I, BssHII, BstUI, Cfr10I, ClaI, CpoI, Eco52I, HaeII, HapII, HhaI, Hin6I, HpaII, HpyCH4IV, MluI, MspI, NaeI, NotI, NruI, NsbI, PmaCI, Psp1406I, PvuI, SacII, SalI, SmaI, and SnaBI. In some embodiments, at least two methylation-sensitive nucleases are used. In some embodiments, at least three methylation-sensitive nucleases are used. In some embodiments, the methylation-sensitive nucleases comprise BstUI and HpaII. In some embodiments, the two methylation-sensitive nucleases comprise HhaI and AccII. In some embodiments, the methylation-sensitive nucleases comprise BstUI, HpaII and Hin6I.

In some embodiments, FspEI is used for digesting the nucleic acid molecules in at least one subsample (e.g., a hypomethylated partition). In some embodiments, BstUI, HpaII and Hin6I are used for digesting the nucleic acid molecules in at least one subsample (e.g., a hypermethylated partition) and FspEI is used for digesting the nucleic acid molecules in at least one other subsample (e.g., a hypomethylated partition). In embodiments involving an intermediately methylated partition, the nucleic acid molecules therein may be digested with a methylation-sensitive nuclease or a methylation-dependent nuclease. In some embodiments, the nucleic acid molecules in an intermediately methylated partition are digested with the same nuclease(s) as the hypermethylated partition. For example, the intermediately methylated partition may be pooled with the hypermethylated partition and then the pooled partitions may be subjected to digestion. In some embodiments, the nucleic acid molecules in an intermediately methylated partition are digested with the same nuclease(s) as the hypomethylated partition. For example, the intermediately methylated partition may be pooled with the hypomethylated partition and then the pooled partitions may be subjected to digestion.

In some embodiments, a subsample is contacted with a nuclease as described above after a step of tagging or attaching adapters to both ends of the DNA. The tags or adapters can be resistant to cleavage by the nuclease using any of the approaches described above. In this approach, cleavage can prevent the nonspecifically partitioned molecule from being carried through the analysis because the cleavage products lack tags or adapters at both ends.

Alternatively, a step of tagging or attaching adapters can be performed after cleavage with a nuclease as described above. Cleaved molecules can be then identified in sequence reads based on having an end (point of attachment to tag or adapter) corresponding to a nuclease recognition site. Processing the molecules in this way can also allow the acquisition of information from the cleaved molecule, e.g., observation of somatic mutations. When tagging or attaching adapters after contacting the subsample with a nuclease, and low molecular weight DNA such as cfDNA is being analyzed, it may be desirable to remove high molecular weight DNA (such as contaminating genomic DNA) from the sample before the contacting step. It may also be desirable to use nucleases that can be heat-inactivated at a relatively low temperature (e.g., 65° C. or less, or 60° C. or less) to avoid denaturing DNA, in that denaturation may interfere with subsequent ligation steps.

Where a sample is partitioned into three subsamples, including a third subsample containing intermediately methylated molecules, the third subsample is in some embodiments contacted with a methylation-sensitive nuclease. Such a step may have any of the features described elsewhere herein with respect to contacting steps, and may be performed before or after a step of tagging or attaching adapters as discussed herein. In some embodiments, the first and third subsamples are combined before being contacted with a methylation-sensitive nuclease. Such a step may have any of the features described elsewhere herein with respect to contacting steps, and may be performed before or after a step of tagging or attaching adapters as discussed above. In some embodiments, the first and third subsamples are differentially tagged before being combined.

Alternatively, where a sample is partitioned into three subsamples, including a third subsample containing intermediately methylated molecules, the third subsample is in some embodiments contacted with a methylation-dependent nuclease. Such a step may have any of the features described elsewhere herein with respect to contacting steps, and may be performed before or after a step of tagging or attaching adapters as discussed above. In some embodiments, the second and third subsamples are combined before being contacted with a methylation-dependent nuclease. Such a step may have any of the features described elsewhere herein with respect to contacting steps, and may be performed before or after a step of tagging or attaching adapters as discussed above. In some embodiments, the second and third subsamples are differentially tagged before being combined.

In some embodiments, the DNA is purified after being contacted with the nuclease, e.g., using SPRI beads. Such purification may occur after heat inactivation of the nuclease. Alternatively, purification can be omitted; thus, for example, a subsequent step such as amplification can be performed on the subsample containing heat-inactivated nuclease. In another embodiment, the contacting step can occur in the presence of a purification reagent such as SPRI beads, e.g., to minimize losses associated with tube transfers. After cleavage and heat inactivation, the SPRI beads can be re-used for cleanup by adding molecular crowding reagents (e.g., PEG) and salt.

In some embodiments, where a conversion procedure is performed on a sample or subsample, the subsequent capturing of one or more target region sets (e.g., at least an epigenetic target region set) from that sample or subsample uses capture probes that comprise probes specific for a modification state (e.g., of at least one base in the sequence to which the probe hybridizes), e.g., complementary to target sequences that have undergone conversion (e.g., conversion of modified or unmodified cytosines to uracils or analogs thereof, such as DHU, that preferentially pair with adenine) or that have not undergone conversion, as desired. As such, the probes can be specific for sequences in which a modification of interest, such as methylation, was or was not present. In some embodiments, where a modification sensitive conversion is performed on a sample or subsample, the subsequent capturing of one or more target region sets (e.g., at least an epigenetic target region set) from that sample or subsample uses capture probes that comprise probes that can hybridize to target sequences regardless of modification state (e.g., comprise a promiscuously pairing nucleobase at a position that may or may not have undergone conversion of modified or unmodified cytosines to uracils or analogs thereof, such as DHU, that preferentially pair with adenine; for example, inosine can pair with C or U).

In some embodiments, the methods comprise preparing a pool comprising at least a portion of the DNA of the second subsample (also referred to as the hypomethylated partition) and at least a portion of the DNA of the first subsample (also referred to as the hypermethylated partition). Target regions, e.g., including epigenetic target regions and/or sequence-variable target regions, may be captured from the pool. The steps of capturing a target region set from at least a portion of a subsample described elsewhere herein encompass capture steps performed on a pool comprising DNA from the first and second subsamples. A step of amplifying DNA in the pool may be performed before capturing target regions from the pool. The capturing step may have any of the features described elsewhere herein.

The epigenetic target regions may show differences in methylation levels and/or fragmentation patterns depending on whether they originated from a tumor or from healthy cells, or what type of tissue they originated from, as discussed elsewhere herein. The sequence-variable target regions may show differences in sequence depending on whether they originated from a tumor or from healthy cells.

Analysis of epigenetic target regions from the hypomethylated partition may be less informative in some applications than analysis of sequence-variable target-regions from the hypermethylated and hypomethylated partitions and epigenetic target regions from the hypermethylated partition. As such, in methods where sequence-variable target-regions and epigenetic target regions are being captured, the latter may be captured to a lesser extent than one or more of the sequence-variable target-regions from the hypermethylated and hypomethylated partitions and epigenetic target regions from the hypermethylated partition. For example, sequence-variable target regions can be captured from the portion of the hypomethylated partition not pooled with the hypermethylated partition, and the pool can be prepared with some (e.g., a majority, substantially all, or all) of the DNA from the hypermethylated partition and none or some (e.g., a minority) of the DNA from the hypomethylated partition. Such approaches can reduce or eliminate sequencing of epigenetic target regions from the hypomethylated partition, thereby reducing the amount of sequencing data that suffices for further analysis.

In some embodiments, including a minority of the DNA of the hypomethylated partition in the pool facilitates quantification of one or more epigenetic features (e.g., methylation or other epigenetic feature(s) discussed in detail elsewhere herein), e.g., on a relative basis.

In some embodiments, the pool comprises a minority of the DNA of the hypomethylated partition, e.g., less than about 50% of the DNA of the hypomethylated partition, such as less than or equal to about 45%, 40%, 35%, 30%, 25%, 20%, 15%, 10%, or 5% of the DNA of the hypomethylated partition. In some embodiments, the pool comprises about 5%-25% of the DNA of the hypomethylated partition. In some embodiments, the pool comprises about 10%-20% of the DNA of the hypomethylated partition. In some embodiments, the pool comprises about 10% of the DNA of the hypomethylated partition. In some embodiments, the pool comprises about 15% of the DNA of the hypomethylated partition. In some embodiments, the pool comprises about 20% of the DNA of the hypomethylated partition.

In some embodiments, the pool comprises a portion of the hypermethylated partition, which may be at least about 50% of the DNA of the hypermethylated partition. For example, the pool may comprise at least about 55%, 60%, 65%, 70%, 75%, 80%, 85%, 90%, or 95% of the DNA of the hypermethylated partition. In some embodiments, the pool comprises 50-55%, 55-60%, 60-65%, 65-70%, 70-75%, 75-80%, 80-85%, 85-90%, 90-95%, or 95-100% of the DNA of the hypermethylated partition. In some embodiments, the second pool comprises all or substantially all of the hypermethylated partition.

In some embodiments, the methods comprise preparing a first pool comprising at least a portion of the DNA of the hypomethylated partition. In some embodiments, the methods comprise preparing a second pool comprising at least a portion of the DNA of the hypermethylated partition. In some embodiments, the first pool further comprises a portion of the DNA of the hypermethylated partition. In some embodiments, the second pool further comprises a portion of the DNA of the hypomethylated partition. In some embodiments, the first pool comprises a majority of the DNA of the hypomethylated partition, and optionally and a minority of the DNA of the hypermethylated partition. In some embodiments, the second pool comprises a majority of the DNA of the hypermethylated partition and a minority of the DNA of the hypomethylated partition. In some embodiments involving an intermediately methylated partition, the second pool comprises at least a portion of the DNA of the intermediately methylated partition, e.g., a majority of the DNA of the intermediately methylated partition. In some embodiments, the first pool comprises a majority of the DNA of the hypomethylated partition, and the second pool comprises a majority of the DNA of the hypermethylated partition and a majority of the DNA of the intermediately methylated partition.

In some embodiments, the methods comprise capturing at least a first set of target regions from the first pool, e.g., wherein the first pool is as set forth in any of the embodiments above. In some embodiments, the first set comprises sequence-variable target regions. In some embodiments, the first set comprises hypomethylation variable target regions and/or fragmentation variable target regions. In some embodiments, the first set comprises sequence-variable target regions and fragmentation variable target regions. In some embodiments, the first set comprises sequence-variable target regions, hypomethylation variable target regions and fragmentation variable target regions. A step of amplifying DNA in the first pool may be performed before this capture step. In some embodiments, capturing the first set of target regions from the first pool comprises contacting the DNA of the first pool with a first set of capture probes. In some embodiments, the first set of capture probes comprises target-binding probes specific for the sequence-variable target regions. In some embodiments, the first set of capture probes comprises target-binding probes specific for the sequence-variable target regions, hypomethylation variable target regions and/or fragmentation variable target regions.

In some embodiments, the methods comprise capturing a second set of target regions or plurality of sets of target regions from the second pool, e.g., wherein the first pool is as set forth in any of the embodiments above. In some embodiments, the second plurality comprises epigenetic target regions, such as hypermethylation variable target regions and/or fragmentation variable target regions. In some embodiments, the second plurality comprises sequence-variable target regions and epigenetic target regions, such as hypermethylation variable target regions and/or fragmentation variable target regions. A step of amplifying DNA in the second pool may be performed before this capture step. In some embodiments, capturing the second plurality of sets of target regions from the second pool comprises contacting the DNA of the first pool with a second set of capture probes, wherein the second set of capture probes comprises target-binding probes specific for the sequence-variable target regions and target-binding probes specific for the epigenetic target regions. In some embodiments, the first set of target regions and the second set of target regions are not identical. For example, the first set of target regions may comprise one or more target regions not present in the second set of target regions. Alternatively or in addition, the second set of target regions may comprise one or more target regions not present in the first set of target regions. In some embodiments, at least one hypermethylation variable target region is captured from the second pool but not from the first pool. In some embodiments, a plurality of hypermethylation variable target regions are captured from the second pool but not from the first pool. In some embodiments, the first set of target regions comprises sequence-variable target regions and/or the second set of target regions comprises epigenetic target regions. In some embodiments, the first set of target regions comprises sequence-variable target regions, and fragmentation variable target regions; and the second set of target regions comprises epigenetic target regions, such as hypermethylation variable target regions and fragmentation variable target regions. In some embodiments, the first set of target regions comprises sequence-variable target regions, fragmentation variable target regions, and comprises hypomethylation variable target regions; and the second set of target regions comprises epigenetic target regions, such as hypermethylation variable target regions and fragmentation variable target regions.

In some embodiments, the first pool comprises a majority of the DNA of the hypomethylated partition and a portion of the DNA of the hypermethylated partition (e.g., about half), and the second pool comprises a portion of the DNA of the hypermethylated partition (e.g., about half). In some such embodiments, the first set of target regions comprises sequence-variable target regions and/or the second set of target regions comprises epigenetic target regions. The sequence-variable target regions and/or the epigenetic target regions may be as set forth in any of the embodiments described elsewhere herein.

In some embodiments, the partitions of DNA are desalted and concentrated in preparation for enzymatic steps of library preparation. Sequences that comprise structural variations may tend to be hypomethylated. Accordingly, in some embodiments, the DNA contacted with a plurality of primers comprising at least two primers that anneal in an antiparallel orientation to a rearranged sequence of DNA is from or comprises at least a portion of a hypomethylated partition. The DNA from or comprising at least a portion of hypomethylated partition may or may not be combined with DNA from or comprising at least a portion of one or more other partitions, such as an intermediate partition or a hypermethylated partition.

### I. Enriching DNA; Enrichment Moieties; Enriched Set

In some embodiments, methods herein comprise enriching (also known as “capturing”) nucleic acid molecules (such as DNA, RNA, or cDNA prepared from the RNA) comprising sequences present in a target region set for subsequent analysis. Such enrichment or capture may be performed on any sample or subsample described herein using any suitable approach known in the art. Enriching may be performed on one or more subsamples prepared during methods disclosed herein. In some embodiments, DNA is enriched from at least the first subsample. In some embodiments, DNA is enriched from at least the first subsample or the second subsample, e.g., at least the first subsample and the second subsample. In some embodiments, DNA is enriched from a first subsample and/or from a second subsample following dividing of the DNA into a plurality of subsamples. Where a first subsample undergoes a separation step (e.g., separating DNA originally comprising the first nucleobase (e.g., hmC) from DNA not originally comprising the first nucleobase, such as hmC-seal), capturing may be performed on any, any two, or all of the DNA originally comprising the first nucleobase (e.g., hmC), the DNA not originally comprising the first nucleobase, and the second subsample. In some embodiments, the subsamples are differentially tagged (e.g., as described herein) and then pooled before undergoing capture.

In some embodiments, the enriching is performed prior to a sequencing step and/or (a) prior to or after a step of selectively depleting the target nucleic acid comprising the wild-type sequence, the target nucleic acid comprising the converted nucleotide, or the target nucleic acid that does not comprise the converted nucleotide; (b) prior to or after a step of amplifying the selectively digested population of target nucleic acids; (c) prior to or after a step of partitioning the population of target nucleic acids into a plurality of subsamples; and/or (d) prior to or after a step of subjecting the population of target nucleic acids to the conversion-procedure that affects a first nucleobase of the DNA differently from a second nucleobase of the DNA.

In some embodiments, the capturing comprises contacting the DNA with probes specific for such target regions. In some embodiments, the probes comprise an oligonucleotide and a capture moiety, such as biotin or one or more of the other examples noted below. The probes can have sequences selected to tile across a panel of regions, such as genes.

Methods comprising DNA enrichment using probes comprising a capture moiety, such as target-specific probes labeled with biotin, may also comprise a second moiety or binding partner that binds to the capture moiety, such as streptavidin. In some embodiments, an enrichment moiety and binding partner can have higher and lower capture yields for different sets of probes, such as those used to enrich for (capture) a sequence-variable target region set and an epigenetic target region set, respectively, as discussed elsewhere herein. Methods comprising capture moieties are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. In some embodiments, a capture moiety that is attached to an analyte is captured by its binding partner which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

In some embodiments, nonspecifically bound DNA that does not comprise a target region is washed away from the enriched DNA. In some embodiments, DNA is then dissociated from the probes and eluted from the solid support using salt washes or buffers comprising another DNA denaturing agent. In some embodiments, the probes are also eluted from the solid support by, e.g., disrupting the biotin-streptavidin interaction. In some embodiments, enriched DNA is amplified following elution from the solid support. In some such embodiments, DNA comprising adapters is amplified using PCR primers that anneal to the adapters. In some embodiments, enriched DNA is amplified while attached to the solid support. In some such embodiments, the amplification comprises use of a PCR primer that anneals to a sequence within an adapter and a PCR primer that anneals to a sequence within a probe annealed to the target region of the DNA.

In some embodiments, target regions are enriched from an aliquot, portion, or subsample of a sample (e.g., a sample that has undergone attachment of adapters and amplification), while a step of partitioning the DNA may be performed on a separate aliquot, portion, or subsample of the sample. Enriching for or capturing DNA comprising target regions may comprise contacting the DNA with a first or second set of target-specific probes. Such target-specific probes may have any of the features described herein for sets of target-specific probes, including but not limited to, in the embodiments set forth herein and the sections relating to probes herein. Capturing may be performed on one or more subsamples prepared during methods disclosed herein. In some embodiments, DNA is enriched from a first subsample or a second subsample, such as following dividing of the DNA into a plurality of subsamples. In some embodiments, the subsamples are differentially tagged (e.g., as described herein) and then pooled before undergoing enrichment. Exemplary methods for enriching DNA comprising epigenetic and/or sequence-variable target regions can be found in, e.g., WO 2020/160414, which is hereby incorporated by reference.

The enriching step or steps may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization.

In some embodiments, methods described herein comprise enriching for a plurality of target region sets of cfDNA obtained from a subject. The target regions may comprise differences depending on whether they originated from a tumor or from healthy cells or from a certain cell type. For example, the target regions comprising epigenetic target regions may show differences in methylation levels and/or fragmentation patterns depending on whether they originated from a tumor or from healthy cells. Similarly, the target regions comprising sequence-variable target regions may show differences in sequence depending on whether they originated from a tumor or from healthy cells. The enriching step produces an enriched set of cfDNA molecules. In some embodiments, cfDNA molecules corresponding to a sequence-variable target region set are enriched at a greater capture yield in the enriched set of cfDNA molecules than cfDNA molecules corresponding to an epigenetic target region set. In some embodiments, a method described herein comprises contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set.

It can be beneficial to enrich cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell). Although copy number variations such as focal amplifications are somatic mutations, they can be detected by sequencing based on read frequency in a manner analogous to approaches for detecting certain epigenetic changes such as changes in methylation.

In some embodiments, the enriched DNA is amplified. In various embodiments, the methods further comprise sequencing the enriched DNA, e.g., to different degrees of sequencing depth for the epigenetic and sequence-variable target region sets, consistent with the discussion herein. In some embodiments, RNA probes are used. In some embodiments, DNA probes are used. In some embodiments, single stranded probes are used. In some embodiments, double stranded probes are used. In some embodiments, single stranded RNA probes are used. In some embodiments, double stranded DNA probes are used.

In some embodiments, an enriching step is performed with probes for a sequence-variable target region set and probes for an epigenetic target region set in the same vessel at the same time, e.g., the probes for the sequence-variable and epigenetic target region sets and capture probes are in the same composition. This approach provides a relatively streamlined workflow.

Alternatively, the capturing step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions comprising captured DNA corresponding to the sequence-variable target region set and captured DNA corresponding to the epigenetic target region set. The compositions can be processed separately as desired (e.g., to fractionate based on methylation as described elsewhere herein) and recombined in appropriate proportions to provide material for further processing and analysis such as sequencing.

In some embodiments, the DNA is amplified. In some embodiments, amplification is performed before the capturing step. In some embodiments, amplification is performed after the capturing step.

In some embodiments, adapters are included in the DNA. This may be done concurrently with an amplification procedure, e.g., by providing the adapters in a 5′ portion of a primer, e.g., as described above. Alternatively, adapters can be added by other approaches, such as ligation.

In some embodiments, tags, which may be or include barcodes, are included in the DNA. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described above. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

In some embodiments, a collection of target-specific probes is used in methods comprising enriched DNA described herein. In some embodiments, the collection of target-specific probes comprises target-binding probes specific for one or more target region sets. In some embodiments, the capture yield of the target-binding probes specific for the sequence-variable target region set is higher (e.g., at least 2-fold higher) than the capture yield of the target-binding probes specific for the epigenetic target region set. In some embodiments, the collection of target-specific probes is configured to have a capture yield specific for the sequence-variable target region set higher (e.g., at least 2-fold higher) than its capture yield specific for the epigenetic target region set.

### 1. Enriched Set

In some embodiments, an enriched (also known as captured) set of DNA (e.g., cfDNA) is provided. With respect to the disclosed methods, the enriched set of DNA may be provided, e.g., by performing an enriching step, such as after a dividing step as described herein. The enrichedf set may comprise DNA corresponding to a sequence-variable target region set, an epigenetic target region set, or a combination thereof.

In some embodiments, a first target region set is enriched from the first subsample, comprising at least epigenetic target regions. The epigenetic target regions enriched from the first subsample may comprise hypermethylation variable target regions. In some embodiments, the hypermethylation variable target regions are CpG-containing regions that are unmethylated or have low methylation in cfDNA from healthy subjects (e.g., below-average methylation relative to bulk cfDNA). In some embodiments, the hypermethylation variable target regions are regions that show lower methylation in healthy cfDNA than in at least one other tissue type. Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypermethylation variable target regions in the first subsample can be an indicator of the presence (or recurrence, depending on the history of the subject) of cancer.

In some embodiments, a second target region set is enriched from the second subsample, comprising at least epigenetic target regions. The epigenetic target regions may comprise hypomethylation variable target regions. In some embodiments, the hypomethylation variable target regions are CpG-containing regions that are methylated or have high methylation in cfDNA from healthy subjects (e.g., above-average methylation relative to bulk cfDNA). In some embodiments, the hypomethylation variable target regions are regions that show higher methylation in healthy cfDNA than in at least one other tissue type. Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypomethylation variable target regions in the second subsample can be an indicator of the presence (or recurrence, depending on the history of the subject) of cancer.

In some embodiments the quantity of enriched sequence-variable target region DNA is greater than the quantity of the enriched epigenetic target region DNA, when normalized for the difference in the size of the targeted regions (footprint size).

Alternatively, first and second enriched sets may be provided, comprising, respectively, DNA corresponding to a sequence-variable target region set and DNA corresponding to an epigenetic target region set. The first and second enriched sets may be combined to provide a combined enriched set.

In some embodiments in which an enriched set comprising DNA corresponding to the sequence-variable target region set and the epigenetic target region set includes a combined enriched set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80- to 90-fold greater concentration, or a 90- to 100-fold greater concentration. The degree of difference in concentrations accounts for normalization for the footprint sizes of the target regions, as discussed in the definition section.

### J. Amplification

In some embodiments, DNA (or cDNA prepared from RNA) is amplified. For example, DNA flanked by adapters added to the DNA as described herein can be amplified by PCR or other amplification methods. Amplification methods of use herein can include any suitable methods, such as known to those of ordinary skill in the art. In some embodiments, amplification is primed by primers binding to primer binding sites in adapters flanking a DNA molecule to be amplified. Amplification methods can involve cycles of denaturation, annealing and extension, resulting from thermocycling, such as polymerase chain reaction (PCR), or can be isothermal, such as in linear amplification methods, transcription-mediated amplification, recombinase polymerase amplification (RPA), helices dependent amplification (HDA), loop-mediated isothermal amplification (LAMP) (Notomi et al., Nuc. Acids Res., 28, e63, 2000), rolling-circle amplification (RCA) (Blanco et al., J. Biol. Chem., 264, 8935-8940, 1989), or hyperbranched rolling circle amplification (Lizard et al., Nat. Genetics, 19, 225-232, 1998). Other amplification methods include the ligase chain reaction, strand displacement amplification, nucleic acid sequence based amplification, and self-sustained sequence based replication.

In some embodiments, detecting the presence or absence of one or more DNA sequences comprises amplification, such as embodiments comprising qPCR or digital PCR. Some such embodiments comprising targeted detection of DNA sequences using qPCR or digital PCR do not comprise standard DNA library preparation steps, such as adapter ligation or tagging.

In some embodiments, dsDNA ligations with T-tailed and C-tailed adapters can be performed, which result in amplification of at least 50, 60, 70 or 80% of double stranded nucleic acids before linking to adapters.

In some embodiments, sample nucleic acids are amplified before sequencing. Amplification may be before and/or after the conversion step. Amplification may in some cases be before one or more capture steps. In some embodiments, the ligating occurs before or simultaneously with amplification.

In some embodiments, the DNA (or cDNA) molecules are amplified prior to the sequencing and/or (a) prior to or after a step of selectively depleting the target nucleic acid comprising the wild-type sequence, the target nucleic acid comprising the converted nucleotide, or the target nucleic acid that does not comprise the converted nucleotide; (b) prior to the enriching for one or more sets of target regions of DNA; (c) prior to or after a step of partitioning the population of target nucleic acids into a plurality of subsamples; and/or (d) prior to or after a step of subjecting the population of target nucleic acids to the conversion-procedure that affects a first nucleobase of the DNA differently from a second nucleobase of the DNA.

### K. Sequencing

In general, sample nucleic acids flanked by adapters with or without prior amplification can be subject to sequencing. Sequencing methods include, for example, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLiD, Ion Torrent, or Nanopore platforms. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously.

In some embodiments, DNA is sequenced in a modification-sensitive manner (i.e., detecting and/or distinguishing unmodified and modified nucleobases). For example, long-read sequencing (also referred to herein as third generation sequencing) methods include those that can generate longer sequencing reads, such as reads in excess of 10 kilobases, as compared to short-read sequencing methods, which generally produce reads of up to about 600 bases in length. Compared to short reads, long reads can improve de novo assembly, transcript isoform identification, and detection and/or mapping of structural variants. Furthermore, long-read sequencing of native DNA or RNA molecules reduces amplification bias and preserves base modifications, such as methylation status. Long-read sequencing technologies useful herein can include any suitable long-read sequencing methods, including, but not limited to, Pacific Biosciences (PacBio) single-molecule real-time (SMRT) sequencing, Oxford Nanopore Technologies (ONT) nanopore sequencing, and synthetic long-read sequencing approaches, such as linked reads, proximity ligation strategies, and optical mapping. Synthetic long-read approaches comprise assembly of short reads from the same DNA molecule to generate synthetic long reads, and may be used in conjunction with “true” long-read sequencing technologies, such as SMRT and nanopore sequencing methods.

Single-molecule real-time (SMRT) sequencing facilitates direct detection of, e.g., 5-methylcytosine and 5-hydroxymethylcytosine as well as unmodified cytosine (Weirather J L, et al., “Comprehensive comparison of Pacific Biosciences and oxford Nanopore Technologies and their applications to transcriptome analysis,” F1000Research, 6:100, 2017). Whereas next-generation sequencing methods detect augmented signals from a clonal population of amplified DNA fragments, SMRT sequencing captures a single DNA molecule, maintaining base modification during sequencing. The error rate of raw PacBio SMRT sequencing-generated data is about 13-15%, as the signal-to-noise ratio from single DNA molecules not high. To increase accuracy, this platform uses a circular DNA template by ligating hairpin adaptors to both ends of target double-stranded DNA. As the polymerase repeatedly traverses and replicates the circular molecule, the DNA template is sequenced multiple times to generate a continuous long read (CLR). The CLR can be split into multiple reads (“subreads”) by removing adapter sequences, and multiple subreads generate circular consensus sequence (“CCS”) reads with higher accuracy. The average length of a CLR is >10 kb and up to 60 kb, with length depending on the polymerase lifetime. Thus, the length and accuracy of CCS reads depends on the fragment sizes. PacBio sequencing has been utilized for genome (e.g., de novo assembly, detection of structural variants and haplotyping) and transcriptome (e.g., gene isoform reconstruction and novel gene/isoform discovery) studies.

ONT is a nanopore-based single molecule sequencing technology (Weirather J L, et al., F1000Research, 6:100, 2017). ONT directly sequences a native single-stranded DNA (ssDNA) molecule by measuring characteristic current changes as the bases are threaded through the nanopore by a molecular motor protein. ONT uses a hairpin library structure similar to the PacBio circular DNA template: the DNA template and its complement are bound by a hairpin adaptor. Therefore, the DNA template passes through the nanopore, followed by a hairpin and finally the complement. The raw read can be split into two “1D” reads (“template” and “complement”) by removing the adaptor. The consensus sequence of two “1D” reads is a “2D” read with a higher accuracy.

5-letter and 6-letter sequencing methods include whole genome sequencing methods capable of sequencing A, C, T, and G in addition to 5mC and 5hmC to provide a 5-letter (A, C, T, G, and either 5mC or 5hmC) or 6-letter (A, C, T, G, 5mC, and 5hmC) digital readout in a single workflow. The processing of the DNA sample is entirely enzymatic and avoids the DNA degradation and genome coverage biases of bisulfite treatment. In an exemplary 5-letter sequencing method developed by Cambridge Epigenetix, the sample DNA is first fragmented via sonication and then ligated to short, synthetic DNA hairpin adaptors at both ends (Füllgrabe, et al. 2022, bioRxiv doi: https://doi.org/10.1101/2022.07.08.499285). The construct is then split to separate the sense and antisense sample strands. For each original sample strand a complementary copy strand is synthesized by DNA polymerase extension of the 3′-end to generate a hairpin construct with the original sample DNA strand connected to its complementary strand, lacking epigenetic modifications, via a synthetic loop. Sequencing adapters are then ligated to the end. Modified cytosines are enzymatically protected. The unprotected Cs are then deaminated to uracil, which is subsequently read as thymine. The deaminated constructs are no longer fully complementary and have substantially reduced duplex stability, thus the hairpins can be readily opened and amplified by PCR. The constructs can be sequenced in paired-end format whereby read 1 (P1 primed) is the original stand and read 2 (P2 primed) is the copy stand. The read data is pairwise aligned so read 1 is aligned to its complementary read 2. Cognate residues from both reads are computationally resolved to produce a single genetic or epigenetic letter. Pairings of cognate bases that differ from the permissible five are the result of incomplete fidelity at some stage(s) comprising sample preparation, amplification, or erroneous base calling during sequencing. As these errors occur independently to cognate bases on each strand, substitutions result in a non-permissible pair. Non-permissible pairs are masked (marked as N) within the resolved read and the read itself is retained, leading to minimal information loss and high accuracy at read-level. The resolved read is aligned to the reference genome. Genetic variants and methylation counts are produced by read-counting at base-level.

5hmC has been shown to have value as a marker of biological states and disease which includes early cancer detection from cell-free DNA. In adapting 5-letter to 6-letter sequencing, 5mC is disambiguated from 5hmC without compromising genetic base calling within the same sample fragment. The first three steps of the workflow are identical to 5-letter sequencing described above, to generate the adapter ligated sample fragment with the synthetic copy strand. Methylation at 5mC is enzymatically copied across the CpG unit to the C on the copy strand, whilst 5hmC is enzymatically protected from such a copy. Thus, unmodified C, 5mC and 5hmC in each of the original CpG units are distinguished by unique 2-base combinations. The unmodified cytosines are then deaminated to uracil, which is subsequently read as thymine. The DNA is subjected to PCR amplification and sequencing as described earlier. The reads are pairwise aligned and resolved using a 2-base code. Each of unmodified C, 5mC, and 5hmC can be resolved as the three CpG units are distinct sequencing environments of the 2-base code.

In some embodiments, the sequencing comprises targeted sequencing in which one or more genomic regions of interest are sequenced. In some such embodiments, the genomic regions of interest comprise regions present one or more genes selected from Tables 1, 2, 3, 4, and/or 5. In some such embodiments, DNA sequences that do not comprise regions of interest are not sequenced. Some embodiments comprise non-targeted sequencing, e.g., all genomic regions of the DNA in a treated sample or subsample are sequenced, or genomic regions are randomly chosen for sequencing. In other embodiments, detecting DNA the presence or absence of sequences comprises sequencing DNA that is not enriched for genomic regions of interest (non-targeted sequencing), e.g., wherein detectable sequences are obtained in a substantially unbiased manner.

In some embodiments, a sequencing step is performed on a library comprising captured set of target regions, which may comprise any of the target region sets described herein. In some embodiments, a sequencing step is performed on a library comprising a subsample that has not undergone capture/enrichment (e.g., a whole genome subsample). For example, target regions may be captured from the first subsample and the second sample and then sequenced; or target regions may be captured from the first subsample and combined with the second subsample after processing such as contacting and tagging steps; or target regions may be captured from the second subsample and combined with the first subsample after processing such as contacting and tagging steps; or both the first and second subsamples may be processed and combined without undergoing capture/enrichment.

The sequencing reactions can be performed on one or more forms of nucleic acids at least one of which is known to contain markers of cancer or of other disease. The sequencing reactions can also be performed on any nucleic acid fragments present in the sample. In some embodiments, sequence coverage of the genome may be less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of at least 5%, 10%, 15%, 20%, 25%, 30%, 40%, 50%, 60%, 70%, or 80% of the genome. Sequence coverage can performed on at least 5, 10, 20, 70, 100, 200 or 500 different genes, or at most 5000, 2500, 1000, 500 or 100 different genes.

Simultaneous sequencing reactions may be performed using multiplex sequencing. In some cases, cell-free nucleic acids may be sequenced with at least 1000, 2000, 3000, 4000, 5000, 6000, 7000, 8000, 9000, 10000, 50000, 100,000 sequencing reactions. In other cases cell-free nucleic acids may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base).

In some embodiments, the sequencing comprises generating a plurality of sequencing reads and mapping the plurality of sequencing reads to one or more reference sequences (such as one or more human reference sequences) to generate mapped sequence reads. In some embodiments, at least a portion of the DNA, RNA, or cDNA generated from the RNA of at least a first and second subsample is sequenced in the same sequencing cell.

### 2. Differential Depth of Sequencing

In some embodiments, nucleic acids corresponding to the sequence-variable target region set are sequenced to a greater depth of sequencing than nucleic acids corresponding to the epigenetic target region set. In some embodiments, nucleic acids corresponding to the hydroxymethylation-variable target region set are sequenced to a greater depth of sequencing than nucleic acids corresponding to at least one other target region set. For example, the depth of sequencing for nucleic acids corresponding to the sequence-variable and/or hydroxymethylation-variable target region sets may be at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold greater, or 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, 14- to 15-fold, or 15- to 100-fold greater, than the depth of sequencing for nucleic acids corresponding to the epigenetic target region set or to at least one other target region set. In some embodiments, said depth of sequencing is at least 2-fold greater. In some embodiments, said depth of sequencing is at least 5-fold greater. In some embodiments, said depth of sequencing is at least 10-fold greater. In some embodiments, said depth of sequencing is 4- to 10-fold greater. In some embodiments, said depth of sequencing is 4- to 100-fold greater. Each of these embodiments refer to the extent to which nucleic acids corresponding to the sequence-variable target region set are sequenced to a greater depth of sequencing than nucleic acids corresponding to the epigenetic target region set.

In some embodiments, the captured DNA corresponding to the sequence-variable target region set and the captured DNA corresponding to the epigenetic target region set are sequenced concurrently, e.g., in the same sequencing cell (such as the flow cell of an Illumina sequencer) and/or in the same composition, which may be a pooled composition resulting from recombining separately captured sets or a composition obtained by capturing the DNA corresponding to the sequence-variable target region set and the captured DNA corresponding to the epigenetic target region set in the same vessel.

In some embodiments, the captured DNA corresponding to the hydroxymethylation variable target region set and the captured DNA corresponding to the at least one other target region set are sequenced concurrently, e.g., in the same sequencing cell (such as the flow cell of an Illumina sequencer) and/or in the same composition, which may be a pooled composition resulting from recombining separately captured sets or a composition obtained by capturing the DNA corresponding to the hydroxymethylation variable target region set and the captured DNA corresponding to the at least one other target region set in the same vessel.

### III. Additional Features of Certain Disclosed Methods

### A. Samples

A sample can be any biological sample isolated from a subject. A sample can be a bodily sample. Samples can include body tissues, such as known or suspected solid tumors, whole blood, buffy coat, PBMCs, platelets, serum, plasma, stool, red blood cells, white blood cells or leukocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, preferred body fluids for analysis include body fluids comprising cells, such as whole blood, buffy coat separated from whole blood, PBMCs separated from whole blood, a leukapheresis sample, and/or plasma or serum.

In some embodiments, a population of nucleic acids (such as a population of cell-free nucleic acids, such as cell-free DNA or cell-free RNA) is obtained from a serum, plasma, or blood sample (such as a buffy coat sample or any other sample comprising cells or a blood sample (e.g., a whole blood sample, a leukapheresis sample, or a PBMC sample)) from a subject suspected of having neoplasia, a tumor, precancer, or cancer or previously diagnosed with neoplasia, a tumor, precancer, or cancer. The population includes nucleic acids having varying levels of sequence variation, epigenetic variation, and/or post-replication or transcriptional modifications.

A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines, or biologics. The subject may be in remission. The subject may or may not be diagnosed of being susceptible to cancer or any cancer-associated genetic mutations/disorders.

In some embodiments, the sample comprises plasma. The volume of plasma obtained can depend on the desired read depth for sequenced regions. Exemplary volumes are 0.4-40 mL, 5-20 mL, 10-20 mL, and 3-5 mL. For example, the volume can be 0.5 mL, 1 mL, 2 mL, 3 mL, 4 mL, 5 mL, 6 mL, 7 mL, 8 mL, 9 mL, 10 mL, 20 mL, 30 mL, or 40 mL. A volume of sampled plasma may be 5 to 20 mL. In some embodiments, the sample volume is 3-5 mL of plasma, such as 4 mL of plasma, per 10 mL whole blood.

In some embodiments, the sample comprises whole blood. Exemplary volumes of sampled whole blood are 0.4-40 mL, 5-20 mL, 10-20 mL, 1-6 mL, 1-3 mL, and 3-5 mL. For example, the volume can be 0.5 mL, 1 mL, 2 mL, 3 mL, 4 mL, 5 mL, 6 mL, 7 mL, 8 mL, 9 mL, 10 mL, 20 mL, 30 mL, or 40 mL. A volume of sampled whole blood may be 5 to 20 mL. In some embodiments, the sample volume is 1-5 mL of whole blood, such as 2.5 mL of whole blood.

In some embodiments, the sample comprises buffy coat separated from whole blood. Exemplary volumes of sampled buffy coat are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL 10 mL, or 20 mL. A volume of sampled buffy coat may be 1 to 10 mL. In some embodiments, the sample volume is 0.1-0.5 mL of buffy coat, such as 0.3 mL of buffy coat, per 10 mL whole blood.

In some embodiments, the sample comprises PBMCs separated from whole blood. Exemplary volumes of sampled PBMCs are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL 10 mL, or 20 mL. A volume of sampled PBMCs may be 1 to 10 mL. In some embodiments, the sample volume is 0.1-0.5 mL of PBMCs, such as 0.3 mL of PBMCs, per 10 mL whole blood.

In some embodiments, the sample comprises leukocytes separated from subject blood using leukapheresis. Exemplary volumes of sampled leukocytes from leukapheresis are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL, 10 mL, or 20 mL. A volume of sampled leukocytes from leukapheresis may be 1 to 10 mL. In some embodiments, the sample volume is 0.1-0.6 mL of leukocytes from leukapheresis, such as 0.4 mL of leukocytes, per 10 mL whole blood.

A sample can comprise various amount of nucleic acid that contains genome equivalents. For example, a sample of about 30 ng cDNA can contain about 10,000 (10⁴) haploid human genome equivalents. Similarly, a sample of about 100 ng of cDNA can contain about 30,000 haploid human genome equivalents.

A sample can comprise nucleic acids from different sources, e.g., from cells of the same subject or from cells of different subjects. A sample can comprise nucleic acids carrying mutations. For example, a sample can comprise cDNA carrying germline mutations and/or somatic mutations. Germline mutations refer to mutations existing in germline nucleic acids of a subject. Somatic mutations refer to mutations originating in somatic cells of a subject, e.g., precancer cells or cancer cells. A sample can comprise cDNA carrying cancer-associated mutations (e.g., cancer-associated somatic mutations). A sample can comprise an epigenetic variant (i.e., a chemical or protein modification), wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

Exemplary amounts of nucleic acids (e.g., cDNA prepared from RNA from a sample comprising cells or a blood sample (e.g., a whole blood sample, a buffy coat sample, a leukapheresis sample, or a PBMC sample) in a sample before amplification range from about 1 fg to about 1 g, e.g., 1 μg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of nucleic acid molecules. The amount can be up to 1 femtogram (fg), 10 fg, 100 fg, 1 picogram (pg), 10 μg, 100 pg, 1 ng, 10 ng, 100 ng, 150 ng, or 200 ng of nucleic acid molecules. The method can comprise obtaining 1 femtogram (fg) to 200 ng.

Nucleic acids can be isolated from cells or bodily fluids, which may comprise cells. Cells can be lysed and cellular nucleic acids processed. Generally, after addition of buffers and wash steps, nucleic acids can be precipitated with an alcohol. Further clean up steps may be used such as silica-based columns to remove contaminants or salts. Non-specific bulk carrier nucleic acids, such as C 1 DNA, or DNA or protein for hybridization and/or ligation, may be added throughout the reaction to optimize certain aspects of the procedure such as yield.

After such processing, samples can include various forms of nucleic acid including double stranded cDNA, single stranded cDNA, and single stranded RNA. In some embodiments, single stranded cDNA and RNA can be converted to double stranded forms so they are included in subsequent processing and analysis steps.

cDNA molecules can be linked to adapters at either one end or both ends. Typically, double-stranded molecules are blunt ended by treatment with a polymerase with a 5′-3′ polymerase and a 3′-5′ exonuclease (or proof-reading function), in the presence of all four standard nucleotides. Klenow large fragment, T4, Vent, or Deep Vent polymerases are examples of suitable polymerase. The blunt ended cDNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the nucleic acid molecules and the adapter tags have blunt ends. In sticky-end ligation, typically, the nucleic acid molecules bear an “A” overhang and the adapters bear a “T” overhang.

### B. Subjects

In some embodiments, the population of target nucleic acids (e.g., cfDNA or cfRNA, or DNA or RNA from a sample comprising cells or a blood sample (e.g., a whole blood sample, a buffy coat sample, a leukapheresis sample, or a PBMC sample)) is obtained from a subject having a cancer or a precancer, an infection, transplant rejection, or other disease directly or indirectly affecting the immune system. In some embodiments, the population of target nucleic acids is obtained from a subject suspected of having a cancer or a precancer, an infection, transplant rejection, or other disease directly or indirectly affecting the immune system. In some embodiments, the population of target nucleic acids is obtained from a subject having a tumor. In some embodiments, the population of target nucleic acids is obtained from a subject suspected of having a tumor. In some embodiments, the population of target nucleic acids is obtained from a subject having neoplasia. In some embodiments, the population of target nucleic acids is obtained from a subject suspected of having neoplasia. In some embodiments, the population of target nucleic acids is obtained from a subject in remission from a tumor, cancer, or neoplasia (e.g., following chemotherapy, surgical resection, radiation, or a combination thereof). In any of the foregoing embodiments, the precancer, cancer, tumor, or neoplasia or suspected precancer, cancer, tumor, or neoplasia may be of the bladder, head or neck, lung, colon, rectum, kidney, breast, prostate, skin, or liver. In some embodiments, the precancer, cancer, tumor, or neoplasia or suspected precancer, cancer, tumor, or neoplasia is of the lung. In some embodiments, the precancer, cancer, tumor, or neoplasia or suspected precancer, cancer, tumor, or neoplasia is of the colon or rectum. In some embodiments, the precancer, cancer, tumor, or neoplasia or suspected precancer, cancer, tumor, or neoplasia is of the breast. In some embodiments, the precancer, cancer, tumor, or neoplasia or suspected precancer, cancer, tumor, or neoplasia is of the prostate. In any of the foregoing embodiments, the subject may be a human subject.

### C. Capture Moieties

As discussed above, nucleic acids in a sample can be subject to a capture step, in which molecules having target regions are captured for subsequent analysis. Target capture can involve use of probes (e.g., oligonucleotides) labeled with a capture moiety, such as biotin, and a second moiety or binding partner that binds to the capture moiety, such as streptavidin. In some embodiments, a capture moiety and binding partner can have higher and lower capture yields for different sets of target regions, such as those of the sequence-variable target region set and the epigenetic target region set, respectively, as discussed elsewhere herein. Methods comprising capture moieties are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

### D. Target Region Sets

In some embodiments, certain genomic regions of interest are detected and/or enriched. The genomic regions of interest may comprise one or more target region sets. In some embodiments, target region sets comprise variations that are not prevalent in DNA from healthy subjects or not prevalent in DNA obtained from healthy tissue regions. In some embodiments, target region sets comprise variations present in healthy cells but not normally present in the sample type, such as a blood sample. In some embodiments, the variations are present in aberrant cells (e.g., hyperplastic, metaplastic, or neoplastic cells). Exemplary target region sets include sequence-variable target region sets, epigenetic target region sets.

In some embodiments, a first target region set is detected, comprising at least epigenetic target regions. In some embodiments, the epigenetic target regions detected in a first subsample comprise hypermethylation variable target regions. In some embodiments, the hypermethylation variable target regions are CpG-containing regions that are unmethylated or have low methylation in DNA (such as cfDNA) from healthy subjects (e.g., below-average methylation relative to bulk cfDNA). In some embodiments, the hypermethylation variable target regions show type-specific hypermethylation in healthy DNA (such as cfDNA) from one or more related cell or tissue types. Without wishing to be bound by any particular theory, the presence of cancer cells may increase the shedding of DNA into the bloodstream (e.g., from the cancer and/or the surrounding tissue). As such, the distribution of tissue of origin of cfDNA may change upon carcinogenesis. Thus, an increase in the level of hypermethylation variable target regions in the first subsample can be an indicator of the presence (or recurrence, depending on the history of the subject) of cancer.

In some embodiments, the methods herein comprise detecting a second captured target region set from a sample or second subsample, comprising at least epigenetic target regions. In some embodiments, the second epigenetic target region set comprises hypomethylation variable target regions. In some embodiments, the hypomethylation variable target regions are CpG-containing regions that are methylated or have high methylation in DNA (such as cfDNA) from healthy subjects (e.g., above-average methylation relative to bulk cfDNA). Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypomethylation variable target regions in the second subsample can be an indicator of the presence (or recurrence, depending on the history of the subject) of cancer.

Additionally, target region sets may comprise DNA corresponding to a sequence-variable target region set.

### 1. Epigenetic Target Region Sets

In some embodiments, a target region set is or comprises an epigenetic target region set. Epigenetic target region sets may comprise one or more types of target regions likely to differentiate DNA from neoplastic (e.g., tumor or cancer) cells and from healthy cells, e.g., non-neoplastic circulating cells. Exemplary types of such regions are discussed in detail herein. The epigenetic target region set may also comprise one or more control regions, e.g., as described herein.

In some embodiments, the epigenetic target region set has a footprint of at least 100 kbp, e.g., at least 200 kbp, at least 300 kbp, or at least 400 kbp. In some embodiments, the epigenetic target region set has a footprint in the range of 100-20 Mbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp, 1.5-2 Mbp, 2-3 Mbp, 3-4 Mbp, 4-5 Mbp, 5-6 Mbp, 6-7 Mbp, 7-8 Mbp, 8-9 Mbp, 9-10 Mbp, or 10-20 Mbp. In some embodiments, the epigenetic target region set has a footprint of at least 20 Mbp.

a. Hypermethylation and Hypomethylation Variable Target Regions

In some embodiments, an epigenetic target region set comprises a hypermethylation variable target region. In some embodiments, the hypermethylation variable target regions are differentially or exclusively hypermethylated in one or more related cell or tissue types. Such hypermethylation variable target regions may be hypermethylated in other cell or tissue types but not to the extent observed in the one or more related cell or tissue types. In some embodiments, the hypermethylation variable target regions show even higher methylation in cfDNA from a diseased cell of the one or more related cell or tissue types.

In some embodiments, hypermethylation variable target regions refer to regions where an increase in the level of observed methylation, e.g., in a cfDNA sample, indicates an increased likelihood that a sample (e.g., of cfDNA) contains DNA produced by neoplastic cells, such as tumor or cancer cells. For example, hypermethylation of promoters of tumor suppressor genes has been observed repeatedly. See, e.g., Kang et al., Genome Biol. 18:53 (2017) and references cited therein. In another example, as discussed above, hypermethylation variable target regions can include regions that do not necessarily differ in methylation in cancerous tissue relative to DNA from healthy tissue of the same type, but do differ in methylation (e.g., have more methylation) relative to cfDNA that is typical in healthy subjects. Where, for example, the presence of a cancer results in increased cell death such as apoptosis of cells of the tissue type corresponding to the cancer, such a cancer can be detected at least in part using such hypermethylation variable target regions.

An extensive discussion of methylation variable target regions in colorectal cancer is provided in Lam et al., Biochim Biophys Acta. 1866:106-20 (2016). These include VIM, SEPT9, ITGA4, OSM4, GATA4 and NDRG4. An exemplary set of hypermethylation variable target regions based on colorectal cancer (CRC) studies is provided in Table 1. Many of these genes likely have relevance to cancers beyond colorectal cancer; for example, TP53 is widely recognized as a critically important tumor suppressor and hypermethylation-based inactivation of this gene may be a common oncogenic mechanism. A gene is considered to comprise a DMR when the DMR is located within an untranslated region (UTR), intron, or exon of the gene, or within 5000 nucleotides of either the 5′ end of the sense strand of the 5′ UTR or the 3′ end of the sense strand of the 3′ UTR.

TABLE 1
Exemplary Hypermethylation Target
Regions based on CRC studies.

| | Additional | |
|---|---|---|
| Gene Name | Gene Name | Chromosome |
| VIM | | chr10 |
| SEPT9 | | chr17 |
| CYCD2 | CCND2 | chr12 |
| TFPI2 | | chr7 |
| GATA4 | | chr8 |
| RARB2 | RARB | chr3 |
| p16INK4a | CDKN2A | chr9 |
| MGMT | | chr10 |
| APC | | chr5 |
| NDRG4 | | chr16 |
| HLTF | | chr3 |
| HPP1 | TMEFF2 | chr2 |
| hMLH1 | MLH1 | chr3 |
| RASSF1A | RASSF1 | chr3 |
| CDH13 | | chr16 |
| IGFBP3 | | chr7 |
| ITGA4 | | chr2 |

In some embodiments, genomic regions targeted for sequencing comprise a plurality of loci listed in Table 1, e.g., at least 10%, 20%, 30%, 40%, 50%, 60%, 70%, 80%, 90%, or 100% of the loci listed in Table 1. In some embodiments, genomic regions are captured using probes. For example, for each locus included as a target region, there may be one or more probes with a hybridization site that binds between the transcription start site and the stop codon (the last stop codon for genes that are alternatively spliced) of the gene, or in the promoter region of the gene. In some embodiments, the one or more probes bind within 300 bp of the transcription start site of a gene in Table 1, e.g., within 200 or 100 bp.

Methylation variable target regions in various types of lung cancer are discussed in detail, e.g., in Ooki et al., Clin. Cancer Res. 23:7141-52 (2017); Belinksy, Annu. Rev. Physiol. 77:453-74 (2015); Hulbert et al., Clin. Cancer Res. 23:1998-2005 (2017); Shi et al., BMC Genomics 18:901 (2017); Schneider et al., BMC Cancer. 11:102 (2011); Lissa et al., Transl Lung Cancer Res 5(5):492-504 (2016); Skvortsova et al., Br. J. Cancer. 94(10):1492-1495 (2006); Kim et al., Cancer Res. 61:3419-3424 (2001); Furonaka et al., Pathology International 55:303-309 (2005); Gomes et al., Rev. Port. Pneumol. 20:20-30 (2014); Kim et al., Oncogene. 20:1765-70 (2001); Hopkins-Donaldson et al., Cell Death Differ. 10:356-64 (2003); Kikuchi et al., Clin. Cancer Res. 11:2954-61 (2005); Heller et al., Oncogene 25:959-968 (2006); Licchesi et al., Carcinogenesis. 29:895-904 (2008); Guo et al., Clin. Cancer Res. 10:7917-24 (2004); Palmisano et al., Cancer Res. 63:4620-4625 (2003); and Toyooka et al., Cancer Res. 61:4556-4560, (2001).

An exemplary set of hypermethylation variable target regions based on lung cancer studies is provided in Table 2. Many of these genes likely have relevance to cancers beyond lung cancer; for example, Casp8 (Caspase 8) is a key enzyme in programmed cell death and hypermethylation-based inactivation of this gene may be a common oncogenic mechanism not limited to lung cancer. Additionally, a number of genes appear in both Tables 1 and 2, indicating generality.

TABLE 2
Exemplary Hypermethylation Target Regions
based on Lung Cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

Any of the foregoing embodiments concerning target regions identified in Table 2 may be combined with any of the embodiments described above concerning target regions identified in Table 1. In some embodiments, genomic regions targeted for sequencing comprise a plurality of loci listed in Table 1 or Table 2, e.g., at least 10%, 20%, 30%, 40%, 50%, 60%, 70%, 80%, 90%, or 100% of the loci listed in Table 1 or Table 2.

Additional hypermethylation target regions may be obtained, e.g., from the Cancer Genome Atlas. Kang et al., Genome Biology 18:53 (2017), describe construction of a probabilistic method called CancerLocator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

In some embodiments, an epigenetic target region set comprises a hypomethylation variable target region. In some embodiments, the hypomethylation variable target regions are exclusively hypomethylated in one or more related cell or tissue types. Such hypomethylation variable target regions may be hypomethylated in other cell or tissue types but not to the extent observed in the one or more related cell or tissue types.

In some embodiments, where different epigenetic target regions are captured, the epigenetic target regions comprise hypermethylation and/or hypomethylation variable target regions.

Further exemplary hypermethylation variable target regions and hypomethylation variable target regions useful for distinguishing between various cell types have been identified by analyzing DNA obtained from various cell types via whole gnome bisulfite sequencing, as described, e.g., in Scott, C. A., Duryea, J. D., MacKay, H. et al., “Identification of cell type-specific methylation signals in bulk whole genome bisulfite sequencing data,” Genome Biol 21, 156 (2020) (doi.org/10.1186/s13059-020-02065-5). Whole-genome bisulfite sequencing data is available from the Blueprint consortium, available on the internet at dec.blueprint-epigenome.eu.

b. CTCF Binding Regions

In some embodiments, an epigenetic target region set comprises CTCF binding regions. CTCF is a DNA-binding protein that contributes to chromatin organization and often colocalizes with cohesin. Perturbation of CTCF binding sites has been reported in a variety of different cancers. See, e.g., Katainen et al., Nature Genetics, doi:10.1038/ng.3335, published online 8 Jun. 2015; Guo et al., Nat. Commun. 9:1520 (2018). CTCF binding results in recognizable patterns in cfDNA that can be detected by sequencing, e.g., through fragment length analysis. Thus, perturbations of CTCF binding result in variation in the fragmentation patterns of cfDNA. As such, CTCF binding sites are a type of fragmentation variable target region.

There are many known CTCF binding sites. See, e.g., the CTCFBSDB (CTCF Binding Site Database), available on the Internet at insulatordb.uthsc.edu/; Cuddapah et al., Genome Res. 19:24-32 (2009); Martin et al., Nat. Struct. Mol. Biol. 18:708-14 (2011); Rhee et al., Cell. 147:1408-19 (2011), each of which are incorporated by reference. Exemplary CTCF binding sites are at nucleotides 56014955-56016161 on chromosome 8 and nucleotides 95359169-95360473 on chromosome 13.

In some embodiments, the CTCF binding regions comprise at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, at least some of the CTCF sites can be methylated or unmethylated, wherein the methylation state is correlated with the whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the CTCF binding sites.

c. Transcription Start Sites

In some embodiments, an epigenetic target region set comprises variable transcription start sites. Transcription start sites may show perturbations in neoplastic cells. For example, nucleosome organization at various transcription start sites in healthy cells of the hematopoietic lineage—which contributes substantially to cfDNA in healthy individuals—may differ from nucleosome organization at those transcription start sites in neoplastic cells. This results in different cfDNA patterns that can be detected by sequencing, as discussed generally in Snyder et al., Cell 164:57-68 (2016); WO 2018/009723; and US20170211143A1. In another example, transcription start sites may not necessarily differ epigenetically in cancerous tissue relative to DNA from healthy tissue of the same type, but do differ epigenetically (e.g., with respect to nucleosome organization) relative to DNA that is typical in healthy subjects. Perturbations of transcription start sites also result in variation in the fragmentation patterns of cfDNA. As such, transcription start sites are also a type of fragmentation variable target regions.

Human transcriptional start sites are available from DBTSS (DataBase of Human Transcription Start Sites), available on the Internet at dbtss.hgc.jp and described in Yamashita et al., Nucleic Acids Res. 34(Database issue): D86-D89 (2006), which is incorporated herein by reference. In some embodiments, the transcriptional start sites comprise at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, at least some of the transcription start sites can be methylated or unmethylated, wherein the methylation state is correlated with whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the transcription start sites.

d. Focal Amplifications

Although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set and may comprise one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1.

e. Methylation Control Regions

It can be useful to include control regions to facilitate data validation. In some embodiments, the epigenetic target region set includes control regions that are expected to be methylated or unmethylated in essentially all samples, regardless of whether the DNA is derived from a cancer cell or a normal cell. In some embodiments, the epigenetic target region set includes control hypomethylated regions that are expected to be hypomethylated in essentially all samples. In some embodiments, the epigenetic target region set includes control hypermethylated regions that are expected to be hypermethylated in essentially all samples.

### 2. Sequence-Variable Target Region Sets

In some embodiments, a target region set is or comprises a sequence-variable target region set. Sequence-variable target region sets may comprise one or more types of target regions likely to differentiate DNA from neoplastic (e.g., tumor or cancer) cells and from healthy cells, e.g., non-neoplastic circulating cells. Exemplary types of such regions are discussed in detail herein. The sequence-variable target region set may also comprise one or more control regions, e.g., as described herein. In some embodiments, a sequence-variable target region set comprises a plurality of regions known to undergo somatic mutations in cancer. In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Examples of listings of genomic locations of interest may be found in, e.g., Table 3 and Table 4 herein. In some embodiments, a sequence-variable target region set comprises portions of at least 5, at least 10, at least 15, at least 20, at least 25, at least 30, at least 35, at least 40, at least 45, at least 50, at least 55, at least 60, at least 65, or 70 of the genes of Table 3. In some embodiments, a sequence-variable target region set comprises at least 5, at least 10, at least 15, at least 20, at least 25, at least 30, at least 35, at least 40, at least 45, at least 50, at least 55, at least 60, at least 65, or 70 of the SNVs of Table 3. In some embodiments, a sequence-variable target region set comprises portions of at least 1, at least 2, at least 3, at least 4, at least 5, or 6 of the fusions of Table 3. In some embodiments, a sequence-variable target region set comprises at least portions of at least 1, at least 2, or 3 of the indels of Table 3. In some embodiments, a sequence-variable target region set comprises portions of at least 5, at least 10, at least 15, at least 20, at least 25, at least 30, at least 35, at least 40, at least 45, at least 50, at least 55, at least 60, at least 65, at least 70, or 73 of the genes of Table 4. In some embodiments, a sequence-variable target region set comprises at least 5, at least 10, at least 15, at least 20, at least 25, at least 30, at least 35, at least 40, at least 45, at least 50, at least 55, at least 60, at least 65, at least 70, or 73 of the SNVs of Table 4. In some embodiments, a sequence-variable target region set comprises portions of at least 1, at least 2, at least 3, at least 4, at least 5, or 6 of the fusions of Table 4. In some embodiments, a sequence-variable target region set comprises at least portions of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 4. Each of these genomic locations of interest may be identified as a backbone region or hot-spot region for a given panel. Table 5 shows an example listing of hot-spot genomic locations of interest. In some embodiments, a sequence-variable target region set comprises portions of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 5. Each hot-spot genomic region is listed with the associated gene, chromosome on which it resides, the start and stop position of the genome representing the gene's locus, the length of the gene's locus in base pairs, the exons covered by the gene, and the critical feature (e.g., type of mutation) of a given genomic region of interest.

TABLE 3

| Point Mutations (SNVs) | | | | | | Fusions |
|---|---|---|---|---|---|---|
| AKT1 | ALK | APC | AR | ARAF | ARIDIA | ALK |
| ATM | BRAF | BRCA1 | BRCA2 | CCND1 | CCND2 | FGFR2 |
| CCNE1 | CDH1 | CDK4 | CDK6 | CDKN2A | CDKN2B | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | SRC | STK11 | |
| TERT | TP53 | TSC1 | VHL | | | |

TABLE 4

| Point Mutations (SNVs) | | | | | | Fusions |
|---|---|---|---|---|---|---|
| AKT1 | ALK | APC | AR | ARAF | ARIDIA | ALK |
| ATM | BRAF | BRCA1 | BRCA2 | CCND1 | CCND2 | FGFR2 |
| CCNE1 | CDH1 | CDK4 | CDK6 | CDKN2A | DDR2 | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | MAPK1 | STK11 | |
| TERT | TP53 | TSC1 | VHL | MAPK3 | MTOR | |
| NTRK3 | | | | | | |

TABLE 5

| | | Start | Stop | Length | Exons | |
|---|---|---|---|---|---|---|
| Gene | Chromosome | Position | Position | (bp) | Covered | Critical Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5: 1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 | | |
| | | | | (total | | |
| | | | | target | | |
| | | | | region) | | |
| | | | | 16330 | | |
| | | | | (total | | |
| | | | | probe | | |
| | | | | coverage) | | |

Examples of listings of target regions of interest may also be found in WO 2020/160414, e.g., at Table 4. Additional examples include loci disclosed in Gale et al., PLoS One 13: e0194630 (2018), incorporated herein by reference, which describes a panel of 35 cancer-related gene targets: AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1. In some embodiments, the sequence-variable target region set comprises target regions from at least 10, 20, 30, or 35 cancer-related genes, such as the cancer-related genes listed herein and in WO 2020/160414.

In some embodiments, the sequence-variable target region set has a footprint of at least 50 kbp, e.g., at least 100 kbp, at least 200 kbp, at least 300 kbp, or at least 400 kbp. In some embodiments, the sequence-variable target region set has a footprint in the range of 100-2000 kbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 MVbp or 1.5-2 MVbp. In some embodiments, the sequence-variable target region set has a footprint of at least 2 MVbp.

### E. Collections of Target-Specific Probes

In some embodiments, a collection of target-specific probes is used in methods described herein. In some embodiments, the collection of target-specific probes comprises target-binding probes specific for a sequence-variable target region set and target-binding probes specific for an epigenetic target region set. In some embodiments, the capture yield of the target-binding probes specific for the sequence-variable target region set is higher (e.g., at least 2-fold higher) than the capture yield of the target-binding probes specific for the epigenetic target region set. In some embodiments, the collection of target-specific probes is configured to have a capture yield specific for the sequence-variable target region set higher (e.g., at least 2-fold higher) than its capture yield specific for the epigenetic target region set.

In some embodiments, the capture yield of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the capture yield of the target-binding probes specific for the epigenetic target region set. In some embodiments, the capture yield of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the capture yield of the target-binding probes specific for the epigenetic target region set.

In some embodiments, the collection of target-specific probes is configured to have a capture yield specific for the sequence-variable target region set at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than its capture yield for the epigenetic target region set. In some embodiments, the collection of target-specific probes is configured to have a capture yield specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than its capture yield specific for the epigenetic target region set.

The collection of probes can be configured to provide higher capture yields for the sequence-variable target region set in various ways, including concentration, different lengths and/or chemistries (e.g., that affect affinity), and combinations thereof. Affinity can be modulated by adjusting probe length and/or including nucleotide modifications as discussed below.

In some embodiments, the target-specific probes specific for the sequence-variable target region set are present at a higher concentration than the target-specific probes specific for the epigenetic target region set. In some embodiments, concentration of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the concentration of the target-binding probes specific for the epigenetic target region set. In some embodiments, the concentration of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the concentration of the target-binding probes specific for the epigenetic target region set. In such embodiments, concentration may refer to the average mass per volume concentration of individual probes in each set.

In some embodiments, the target-specific probes specific for the sequence-variable target region set have a higher affinity for their targets than the target-specific probes specific for the epigenetic target region set. Affinity can be modulated in any way known to those skilled in the art, including by using different probe chemistries. For example, certain nucleotide modifications, such as cytosine 5-methylation (in certain sequence contexts), modifications that provide a heteroatom at the 2′ sugar position, and LNA nucleotides, can increase stability of double-stranded nucleic acids, indicating that oligonucleotides with such modifications have relatively higher affinity for their complementary sequences. See, e.g., Severin et al., Nucleic Acids Res. 39: 8740-8751 (2011); Freier et al., Nucleic Acids Res. 25: 4429-4443 (1997); U.S. Pat. No. 9,738,894. Also, longer sequence lengths will generally provide increased affinity. Other nucleotide modifications, such as the substitution of the nucleobase hypoxanthine for guanine, reduce affinity by reducing the amount of hydrogen bonding between the oligonucleotide and its complementary sequence. In some embodiments, the target-specific probes specific for the sequence-variable target region set have modifications that increase their affinity for their targets. In some embodiments, alternatively or additionally, the target-specific probes specific for the epigenetic target region set have modifications that decrease their affinity for their targets. In some embodiments, the target-specific probes specific for the sequence-variable target region set have longer average lengths and/or higher average melting temperatures than the target-specific probes specific for the epigenetic target region set. These embodiments may be combined with each other and/or with differences in concentration as discussed above to achieve a desired fold difference in capture yield, such as any fold difference or range thereof described above.

In some embodiments, the target-specific probes comprise a capture moiety. The capture moiety may be any of the capture moieties described herein, e.g., biotin. In some embodiments, the target-specific probes are linked to a solid support, e.g., covalently or non-covalently such as through the interaction of a binding pair of capture moieties. In some embodiments, the solid support is a bead, such as a magnetic bead.

In some embodiments, the target-specific probes specific for the sequence-variable target region set and/or the target-specific probes specific for the epigenetic target region set are a bait set as discussed above, e.g., probes comprising capture moieties and sequences selected to tile across a panel of regions, such as genes.

In some embodiments, the target-specific probes are provided in a single composition. The single composition may be a solution (liquid or frozen). Alternatively, it may be a lyophilizate.

Alternatively, the target-specific probes may be provided as a plurality of compositions, e.g., comprising a first composition comprising probes specific for the epigenetic target region set and a second composition comprising probes specific for the sequence-variable target region set. These probes may be mixed in appropriate proportions to provide a combined probe composition with any of the foregoing fold differences in concentration and/or capture yield. Alternatively, they may be used in separate capture procedures (e.g., with aliquots of a sample or sequentially with the same sample) to provide first and second compositions comprising captured epigenetic target regions and sequence-variable target regions, respectively.

### 1. Probes Specific for Epigenetic Target Regions

The probes for the epigenetic target region set may comprise probes specific for one or more types of target regions likely to differentiate DNA from neoplastic (e.g., tumor or cancer) cells from healthy cells, e.g., non-neoplastic circulating cells. Exemplary types of such regions are discussed in detail herein, e.g., in the sections above concerning captured sets. The probes for the epigenetic target region set may also comprise probes for one or more control regions, e.g., as described herein.

In some embodiments, the probes for the epigenetic target region set have a footprint of at least 100 kbp, e.g., at least 200 kbp, at least 300 kbp, or at least 400 kbp. In some embodiments, the epigenetic target region set has a footprint in the range of 100-20 Mbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp, 1.5-2 Mbp, 2-3 Mbp, 3-4 Mbp, 4-5 Mbp, 5-6 Mbp, 6-7 Mbp, 7-8 Mbp, 8-9 Mbp, 9-10 Mbp, or 10-20 Mbp. In some embodiments, the epigenetic target region set has a footprint of at least 20 Mbp.

a. Hypermethylation Variable Target Regions

In some embodiments, the probes for the epigenetic target region set comprise probes specific for one or more hypermethylation variable target regions. Hypermethylation variable target regions may also be referred to herein as hypermethylated DMRs (differentially methylated regions). The hypermethylation variable target regions may be any of those set forth above. For example, in some embodiments, the probes specific for hypermethylation variable target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

b. Hypomethylation Variable Target Regions

In some embodiments, the probes for the epigenetic target region set comprise probes specific for one or more hypomethylation variable target regions. Hypomethylation variable target regions may also be referred to herein as hypomethylated DMRs (differentially methylated regions). The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or comprising nucleotides 8403565-8953708 and/or 151104701-151106035 of human chromosome 1.

c. CTCF Binding Regions

In some embodiments, the probes for the epigenetic target region set include probes specific for CTCF binding regions. In some embodiments, the probes specific for CTCF binding regions comprise probes specific for at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, the probes for the epigenetic target region set comprise at least 100 bp, at least 200 bp at least 300 bp, at least 400 bp, at least 500 bp, at least 750 bp, or at least 1000 bp upstream and downstream regions of the CTCF binding sites.

d. Transcription Start Sites

In some embodiments, the probes for the epigenetic target region set include probes specific for transcriptional start sites. In some embodiments, the probes specific for transcriptional start sites comprise probes specific for at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, the probes for the epigenetic target region set comprise probes for sequences at least 100 bp, at least 200 bp, at least 300 bp, at least 400 bp, at least 500 bp, at least 750 bp, or at least 1000 bp upstream and downstream of the transcriptional start sites.

e. Focal Amplifications

As noted above, although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set, as discussed above. In some embodiments, the probes specific for the epigenetic target region set include probes specific for focal amplifications. In some embodiments, the probes specific for focal amplifications include probes specific for one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the probes specific for focal amplifications include probes specific for one or more of at least 2, 3, 4, 5, 6, 7, 8, 9, 10, 11, 12, 13, 14, 15, 16, 17, or 18 of the foregoing targets.

f. Control Regions

It can be useful to include control regions to facilitate data validation. In some embodiments, the probes specific for the epigenetic target region set include probes specific for control methylated regions that are expected to be methylated in essentially all samples. In some embodiments, the probes specific for the epigenetic target region set include probes specific for control hypomethylated regions that are expected to be hypomethylated in essentially all samples.

### 2. Probes Specific for Sequence-Variable Target Regions

The probes for the sequence-variable target region set may comprise probes specific for a plurality of regions known to undergo somatic mutations in cancer. The probes may be specific for any sequence-variable target region set described herein. Exemplary sequence-variable target region sets are discussed in detail herein, e.g., in the sections above concerning captured sets.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb. In some embodiments, the sequence-variable target region probe set has a footprint of at least 50 kbp, e.g., at least 100 kbp, at least 200 kbp, at least 300 kbp, or at least 400 kbp. In some embodiments, the sequence-variable target region probe set has a footprint in the range of 100-2000 kbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp or 1.5-2 Mbp. In some embodiments, the sequence-variable target region set has a footprint of at least 2 Mbp.

In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, or at 70 of the genes of Table 3. In some embodiments, probes specific for the sequence-variable target region set comprise probes specific for the at least 5, at least 10, at least 15, at least 20, at least 25, at least 30, at least 35, at least 40, at least 45, at least 50, at least 55, at least 60, at least 65, or 70 of the SNVs of Table 3. In some embodiments, probes specific for the sequence-variable target region set comprise probes specific for at least 1, at least 2, at least 3, at least 4, at least 5, or 6 of the fusions of Table 3. In some embodiments, probes specific for the sequence-variable target region set comprise probes specific for at least a portion of at least 1, at least 2, or 3 of the indels of Table 3. In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, at least 70, or 73 of the genes of Table 4. In some embodiments, probes specific for the sequence-variable target region set comprise probes specific for at least 5, at least 10, at least 15, at least 20, at least 25, at least 30, at least 35, at least 40, at least 45, at least 50, at least 55, at least 60, at least 65, at least 70, or 73 of the SNVs of Table 4. In some embodiments, probes specific for the sequence-variable target region set comprise probes specific for at least 1, at least 2, at least 3, at least 4, at least 5, or 6 of the fusions of Table 4. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 4. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 5.

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

### F. Computer SYSTEMS

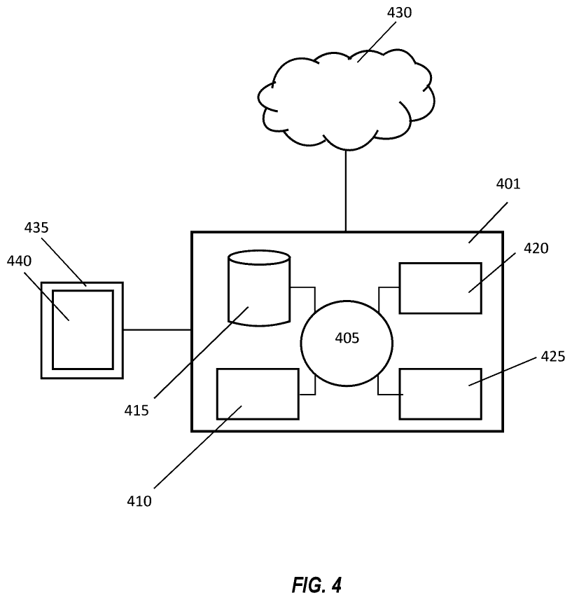

Methods of the present disclosure can be implemented using, or with the aid of, computer systems. FIG. 4 shows a computer system 401 that is programmed or otherwise configured to implement the methods of the present disclosure. The computer system 401 can regulate various aspects sample preparation, sequencing, and/or analysis. In some examples, the computer system 401 is configured to perform sample preparation and sample analysis, including nucleic acid sequencing, e.g., according to any of the methods disclosed herein.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage, and/or electronic display adapters. The memory 410, storage unit 415, interface 420, and peripheral devices 425 are in communication with the CPU 405 through a communication network or bus (solid lines), such as a motherboard. The storage unit 415 can be a data storage unit (or data repository) for storing data. The computer system 401 can be operatively coupled to a computer network 430 with the aid of the communication interface 420. The computer network 430 can be the Internet, an internet and/or extranet, or an intranet and/or extranet that is in communication with the Internet. The computer network 430 in some cases is a telecommunication and/or data network. The computer network 430 can include one or more computer servers, which can enable distributed computing, such as cloud computing. The computer network 430, in some cases with the aid of the computer system 0, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. Examples of operations performed by the CPU 405 can include fetch, decode, execute, and writeback.

The storage unit 415 can store files, such as drivers, libraries, and saved programs. The storage unit 415 can store programs generated by users and recorded sessions, as well as output(s) associated with the programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet. Data may be transferred from one location to another using, for example, a communication network or physical data transfer (e.g., using a hard drive, thumb drive, or other data storage mechanism).

The computer system 401 can communicate with one or more remote computer systems through the network 430. For embodiment, the computer system 401 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine-readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

In an aspect, the present disclosure provides a non-transitory computer-readable medium comprising computer-executable instructions which, when executed by at least one electronic processor, perform at least a portion of a method comprising: (a) contacting a population of target nucleic acids with an oligonucleotide probe, wherein: (i) substantially each target nucleic acid of the population of target nucleic acids is single-stranded and comprises an adapter comprising a restriction enzyme cleavage site and a universal primer binding site; (ii) the population of target nucleic acids comprises a target nucleic acid comprising a wild-type sequence and comprises or is suspected of comprising a target nucleic acid comprising a variant sequence; and (iii) the oligonucleotide probe preferentially forms a substrate for extension with the wild-type sequence relative to the variant sequence; (b) extending the oligonucleotide probe, wherein the extending produces a partially double-stranded target nucleic acid comprising the wild-type sequence; (c) selectively digesting the partially double-stranded target nucleic acid comprising the wild-type sequence, thereby producing a selectively digested population of target nucleic acids; (d) amplifying the selectively digested population of target nucleic acids exponentially, thereby producing a population of amplified target nucleic acids; and (e) sequencing the population of amplified target nucleic acids.

In another aspect, the present disclosure provides a non-transitory computer-readable medium comprising computer-executable instructions which, when executed by at least one electronic processor, perform at least a portion of a method comprising: (a) subjecting a population of target nucleic acids to a conversion procedure that affects a first nucleobase of the target nucleic acids differently from a second nucleobase of the target nucleic acids, wherein (i) substantially each target nucleic acid of the population of target nucleic acids is single-stranded and comprises an adapter comprising a restriction enzyme cleavage site and a universal primer binding site; and (ii) the first nucleobase is a modified or unmodified nucleobase, the second nucleobase is a modified or unmodified nucleobase different from the first nucleobase, and the first nucleobase and the second nucleobase have the same base pairing specificity; thereby providing a population of converted target nucleic acids; (b) contacting the population of converted target nucleic acids with an oligonucleotide probe, wherein the oligonucleotide probe preferentially forms a substrate for extension with a target nucleic acid that comprises a converted nucleotide relative to a target nucleic acid that does not comprise the converted nucleotide; (c) extending the oligonucleotide probe, wherein the extending produces a partially double-stranded target nucleic acid that comprises the converted nucleotide; and (d) selectively digesting the partially double-stranded target nucleic acid that comprises the converted nucleotide, thereby producing a selectively digested population of target nucleic acids; (e) amplifying the selectively digested population of target nucleic acids exponentially, thereby producing a population of amplified target nucleic acids; and (f) sequencing the population of amplified target nucleic acids.

In another aspect, the present disclosure provides a non-transitory computer-readable medium comprising computer-executable instructions which, when executed by at least one electronic processor, perform at least a portion of a method comprising: (a) subjecting a population of target nucleic acids to a conversion procedure that affects a first nucleobase of the target nucleic acids differently from a second nucleobase of the target nucleic acids, wherein (i) substantially each target nucleic acid of the population of target nucleic acids is single-stranded and comprises an adapter comprising a restriction enzyme cleavage site and a universal primer binding site; and (ii) the first nucleobase is a modified or unmodified nucleobase, the second nucleobase is a modified or unmodified nucleobase different from the first nucleobase, and the first nucleobase and the second nucleobase have the same base pairing specificity; thereby providing a population of converted target nucleic acids; (b) contacting the population of converted target nucleic acids with an oligonucleotide probe, wherein the oligonucleotide probe preferentially forms a substrate for extension with a target nucleic acid that does not comprise a converted nucleotide relative to a target nucleic acid that comprises the converted nucleotide; (c) extending the oligonucleotide probe, wherein the extending produces a partially double-stranded target nucleic acid that does not comprise the converted nucleotide; and (d) selectively digesting the partially double-stranded target nucleic acid that does not comprise the converted nucleotide, thereby producing a selectively digested population of target nucleic acids; (e) amplifying the selectively digested population of target nucleic acids exponentially, thereby producing a population of amplified target nucleic acids; and (f) sequencing the population of amplified target nucleic acids.

The code can be pre-compiled and configured for use with a machine have a processer adapted to execute the code or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display that comprises a user interface (UI) for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 1^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

### G. Applications

### 1. Cancer and Other Diseases; Cell Type Quantification

The present methods can be used to diagnose the presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The present disclosure can also be useful in determining the efficacy of a particular treatment option. Successful treatment options may increase the amount of copy number variation, rare mutations, and/or cancer-related epigenetic signatures (such as hypermethylated regions or hypomethylated regions) detected in a subject's blood (such as in DNA isolated from a buffy coat sample or any other sample comprising cells, such as a blood sample (e.g., a whole blood sample, a leukapheresis sample, or a PBMC sample) from the subject) if the treatment is successful as more cancer cells may die and shed DNA, or if a successful treatment results in an increase or decrease in the quantity of a specific immune cell type in the blood and an unsuccessful treatment results in no change. In other examples, this may not occur. In another example, certain treatment options may be correlated with genetic profiles of cancers over time. This correlation may be useful in selecting a therapy for a subject.

Additionally, if a cancer is observed to be in remission after treatment, the present methods can be used to monitor the likelihood of residual disease or the likelihood of recurrence of disease.

In some embodiments, the present methods are used for screening for a cancer, such as a metastasis, or in a method for screening cancer, such as in a method of detecting the presence or absence of a metastasis. For example, the sample can be a sample from a subject who has or has not been previously diagnosed with cancer. In some embodiments, one or more, such as 1, 2, 3, 4, 5, 6, 7, 8, 9, 10, or more samples are collected from a subject as described herein, such as before and/or after the subject is diagnosed with a cancer. In some embodiments, the subject may or may not have cancer. In some embodiments, the subject may or may not have an early-stage cancer. In some embodiments, the subject has one or more risk factors for cancer, such as tobacco use (e.g., smoking), being overweight or obese, having a high body mass index (BMI), being of advanced age, poor nutrition, high alcohol consumption, or a family history of cancer.

In some embodiments, the subject has used tobacco, e.g., for at least 1, 5, 10, or 15 years. In some embodiments, the subject has a high BMI, e.g., a BMI of 25 or greater, 26 or greater, 27 or greater, 28 or greater, 29 or greater, or 30 or greater. In some embodiments, the subject is at least 40, 45, 50, 55, 60, 65, 70, 75, or 80 years old. In some embodiments, the subject has poor nutrition, e.g., high consumption of one or more of red meat and/or processed meat, trans fat, saturated fat, and refined sugars, and/or low consumption of fruits and vegetables, complex carbohydrates, and/or unsaturated fats. High and low consumption can be defined, e.g., as exceeding or falling below, respectively, recommendations in Dietary Guidelines for Americans 2020-2025, available at dietaryguidelines.gov/sites/default/files/2021-03/Dietary_Guidelines_for_Americans-2020-2025.pdf In some embodiments, the subject has high alcohol consumption, e.g., at least three, four, or five drinks per day on average (where a drink is about one ounce or 30 mL of 80-proof hard liquor or the equivalent). In some embodiments, the subject has a family history of cancer, e.g., at least one, two, or three blood relatives were previously diagnosed with cancer. In some embodiments, the relatives are at least third-degree relatives (e.g., great-grandparent, great aunt or uncle, first cousin), at least second-degree relatives (e.g., grandparent, aunt or uncle, or half-sibling), or first-degree relatives (e.g., parent or full sibling).

In some embodiments, the methods and systems disclosed herein may be used to identify customized or targeted therapies to treat a given disease or condition in patients based on the classification of a nucleic acid variant as being of somatic or germline origin. Typically, the disease under consideration is a type of cancer, such as any referred to herein. The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Specific examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

In some embodiments, the cancer is a type of cancer that is not a hematological cancer, e.g., a solid tumor cancer such as a carcinoma, adenocarcinoma, or sarcoma. Type and/or stage of cancer can be detected from genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, such as 5mC and 5mC profiles. Hence, the present methods can in some cases be used in combination with methods used to detect other genetic/epigenetic variations, e.g. in a method of detecting or characterizing a cancer or other methods described herein.

In some embodiments, a method described herein comprises identifying the presence of target regions and/or DNA produced by a tumor (or neoplastic cells, or cancer cells) or by precancer cells. In some embodiments, a method described herein comprises determining the level of target regions and/or identifying the presence of DNA produced by a tumor (or neoplastic cells, or cancer cells) or by precancer cells. In some embodiments, determining the level of target regions comprises determining either an increased level or decreased level of target regions, wherein the increased or decreased level of target regions is determined by comparing the level of target regions with a threshold level/value.

Genetic and/or epigenetic data can also be used for characterizing a specific form of cancer. Cancers are often heterogeneous in both composition and staging. Genetic and/or epigenetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic and/or epigenetic profile of cfDNA derived from the subject, wherein the genetic and/or epigenetic profile comprises a plurality of data resulting from copy number variation and rare mutation analyses. In some embodiments, an abnormal condition is cancer, e.g. as described herein. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease such as where one or more foci (such as one or more tumor foci) are the result of metastases that have spread from a primary site of a cancer. The tissue(s) of origin can be useful for identifying organs affected by the cancer, including the primary cancer and/or metastatic tumors.

The present methods can also be used to quantify levels of different cell types, such as immune cell types, including rare immune cell types, such as activated lymphocytes and myeloid cells at particular stages of differentiation. Such quantification can be based on the numbers of molecules corresponding to a given cell type in a sample. Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer or precancer, or has a metastasis, that is related to changes in proportions of types of immune cells.

The present methods can be used to generate or profile, fingerprint or set of data that is a summation of genetic and/or epigenetic information derived from different cells in a heterogeneous disease. This set of data may comprise copy number variation, epigenetic variation, and mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardiofacial syndrome, WAGR syndrome, Wilson disease, or the like.

In some embodiments, a method described herein comprises detecting a presence or absence of a DNA originating or derived from a tumor cell at a preselected timepoint following a previous cancer treatment of a subject previously diagnosed with cancer using a set of sequence information obtained as described herein. The method may further comprise determining a cancer recurrence score that is indicative of the presence or absence of the DNA originating or derived from the tumor cell for the subject.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

The present methods can also be used to quantify levels of different cell types, such as immune cell types, including rare immune cell types, such as activated lymphocytes and myeloid cells at particular stages of differentiation. Such quantification can be based on the numbers of molecules corresponding to a given cell type in a sample.

Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer, precancer, an infection, transplant rejection, or other diseases or disorder that is related to changes in proportions of types of immune cells. Comparisons of immune cell identities and/or immune cell quantities/proportions between two or more samples collected from a subject at two different time points can allow for monitoring of one or more aspects of a condition in the subject over time, such as a response of the subject to a treatment, the severity of the condition (such as a cancer stage) in the subject, a recurrence of the condition (such as a cancer), and/or the subject's risk of developing the condition (such as a cancer).

The methods discussed above may further comprise any compatible feature or features set forth elsewhere herein, including in the section regarding methods of determining a risk of cancer recurrence in a subject and/or classifying a subject as being a candidate for a subsequent cancer treatment.

2. Methods of Determining a Risk of Cancer Recurrence in a Test Subject and/or Classifying a Subject as being a Candidate for a Subsequent Cancer Treatment

In some embodiments, a method provided herein is or comprises a method of determining a risk of cancer recurrence in a subject. In some embodiments, a method provided herein is or comprises a method of detecting the presence of absence of a metastasis in a subject. In some embodiments, a method provided herein is or comprises a method of classifying a subject as being a candidate for a subsequent cancer treatment.

Any of such methods may comprise collecting a sample (such as DNA, such as DNA originating or derived from a tumor cell) from the subject diagnosed with the cancer at one or more preselected timepoints following one or more previous cancer treatments to the subject. The subject may be any of the subjects described herein. The sample may comprise chromatin, cfDNA, or other cell materials. The sample, such as the DNA sample, may be a tissue sample. The DNA may be DNA, such as cfDNA, from a blood sample (e.g., a whole blood sample, a buffy coat sample, a leukapheresis sample, or a PBMC sample). The DNA may comprise DNA obtained from a tissue sample.

Any of such methods may comprise enriching for a plurality of sets of target regions from DNA from the subject, wherein the plurality of target region sets comprises a sequence-variable target region set and an epigenetic target region set, whereby a captured set of DNA molecules is produced. The enriching step may be performed according to any of the embodiments described elsewhere herein.

In any of such methods, the previous cancer treatment may comprise surgery, administration of a therapeutic composition, and/or chemotherapy.

Any of such methods may comprise sequencing the captured DNA molecules, whereby a set of sequence information is produced. The captured DNA molecules of the sequence-variable target region set may be sequenced to a greater depth of sequencing than the captured DNA molecules of the epigenetic target region set.

Any of such methods may comprise detecting a presence or absence of DNA originating or derived from a tumor cell at a preselected timepoint using the set of sequence information. The detection of the presence or absence of DNA, such as cfDNA, originating or derived from a tumor cell may be performed according to any of the embodiments thereof described elsewhere herein.

Methods of determining a risk of cancer recurrence in a subject may comprise determining a cancer recurrence score that is indicative of the presence or absence, or amount, of the DNA, such as genomic regions of interest and target regions, originating or derived from the tumor cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of detecting the presence or absence of metastasis in a subject may comprise comparing the presence or level of a tissue-specific cell material to the presence or level of the tissue-specific cell material obtained from the subject at a different time, a reference level of the tissue-specific cell material, or to a comparator cell material. Methods herein may comprise additional steps to determine whether a metastasis is present.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, sequence-variable target region sequences are obtained, and determining the cancer recurrence score may comprise determining at least a first subscore indicative of the amount of the levels of particular immune cell types, SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, epigenetic target region sequences are obtained, and determining the cancer recurrence score comprises determining a second subscore indicative of the amount of molecules (obtained from the epigenetic target region sequences) that represent an epigenetic state different from DNA found in a corresponding sample from a healthy subject (e.g., DNA, such as cfDNA, found in a blood sample (e.g., a whole blood sample, a buffy coat sample, a leukapheresis sample, or a PBMC sample) from a healthy subject, or DNA found in a tissue sample from a healthy subject where the tissue sample is of the same type of tissue as was obtained from the subject). These abnormal molecules (i.e., molecules with an epigenetic state different from DNA found in a corresponding sample from a healthy subject) may be consistent with epigenetic changes associated with cancer (such as with a metastasis), e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the target regions, e.g., including one or more of hypermethylation variable target regions, hypomethylation variable target regions, and fragmentation variable target regions (hypermethylation of a hypermethylation variable target region and/or abnormal fragmentation of a fragmentation variable target region may be considered indicative of origination from a tumor cell). This may be done for molecules corresponding to sequence variable target regions, e.g., molecules comprising alterations consistent with cancer, such as SNVs, indels, CNVs, and/or fusions. The fraction of tumor DNA may be determined based on a combination of molecules corresponding to epigenetic target regions and molecules corresponding to sequence variable target regions.

Determination of a cancer recurrence score may be based at least in part on the fraction of tumor DNA, wherein a fraction of tumor DNA greater than a threshold in the range of 10⁻¹¹ to 1 or 10⁻¹⁰ to 1 is sufficient for the cancer recurrence score to be classified as positive for cancer recurrence. In some embodiments, a fraction of tumor DNA greater than or equal to a threshold in the range of 10⁻¹⁰ to 10⁻⁹, 10⁻⁹ to 10⁻⁸, 10⁻⁸ to 10⁻⁷, 10⁻⁷ to 10⁻⁶, 10⁻⁶ to 10⁻⁵, 10⁻⁵ to 10⁻⁴, 10⁻⁴ to 10⁻³, 10⁻³ to 10⁻², or 10⁻² to 10⁻¹ is sufficient for the cancer recurrence score to be classified as positive for cancer recurrence. In some embodiments, the fraction of tumor DNA greater than a threshold of at least 10⁻⁷ is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a subscore indicative of the amount of SNVs, insertions/deletions, CNVs and/or fusions present in sequence-variable target region sequences and a subscore indicative of the amount of abnormal molecules in epigenetic target region sequences, and combining the subscores to provide the cancer recurrence score. Where the subscores are combined, they may be combined by applying a threshold to each subscore independently (e.g., greater than a predetermined number of mutations (e.g., >1) in sequence-variable target regions, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, the set of sequence information comprises sequence-variable target region sequences and epigenetic target region sequences, and determining the cancer recurrence score comprises determining a first subscore indicative of the levels of particular immune cell types, a second subscore indicative of the amount of SNVs, insertions/deletions, CNVs and/or fusions present in sequence-variable target region sequences and a third subscore indicative of the amount of abnormal molecules in epigenetic target region sequences, and combining the first, second, and third subscores to provide the cancer recurrence score. Where the subscores are combined, they may be combined by applying a threshold to each subscore independently in sequence-variable target regions, respectively, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### 3. Methods of Monitoring a Cancer in a Subject Over Time; Sample Collection at Two or More Time Points

In some embodiments, the present methods can be used to monitor one or more aspects of a condition in a subject over time, such as a subject's response to receiving a treatment for a condition (such as a response to a chemotherapeutic or immunotherapeutic), the severity of the condition (such as a cancer stage) in the subject, a recurrence of the condition (such as a cancer), and/or the subject's risk of developing the condition (such as a cancer) and/or to monitor a subject's health as part of a preventative health monitoring program (such as to determine whether and/or when a subject is in need of further diagnostic screening). In some embodiments, monitoring comprises analysis of at least two samples collected from a subject at at least two different time points as described herein.

The methods according to the present disclosure can be useful in predicting a subject's response to a particular treatment option, such as over a period of time. As described elsewhere herein, successful treatment options may increase the amount of cancer associated DNA sequences detected in a subject's blood, such as if the treatment is successful as more cancers may die and shed DNA. In such examples, certain treatment options may be correlated with genetic profiles of cancers over time. This correlation may be useful in selecting a therapy. In some embodiments, successful treatment options may result in an increase or decrease in the levels of different immune cell types (including rare immune cell types), and/or an increase or decrease in the levels of a specific protein or proteins and/or a specific DNA sequence (e.g., of a CDR3), such as in the blood, and an unsuccessful treatment may result in no change. In other examples, this may not occur.

As disclosed herein, in some embodiments, quantities of each of a plurality of cell types, such as immune cell types, are determined based on sequencing and analysis (such as determination of epigenetic and/or genomic signatures) of DNA isolated from at least one sample comprising cells (such as a tissue sample or a blood sample, e.g., a whole blood sample, a buffy coat sample, a leukapheresis sample, or a PBMC sample) from a subject. In some embodiments, differences in levels and/or presence of particular genetic and/or epigenetic signatures in DNA isolated from blood samples from a subject can be used to quantify cell types, such as immune cell types, within the sample. Thus, a comparison of the disclosed genetic and/or epigenetic signatures in DNA isolated from blood samples collected from a subject at two or more time points can be used to monitor changes in cell type quantities in the subject under different conditions (such as prior to and after a treatment), or over time (e.g., as part of a preventative health monitoring program).

The disclosed methods can include evaluating (such as quantifying) and/or interpreting cell types (such as immune cell types) present in one or more samples (such as a tissue sample or a blood sample, e.g., a whole blood sample, a buffy coat sample, a leukapheresis sample, or a PBMC sample) collected from a subject at one or more timepoints in comparison to a selected baseline value or reference standard (or a selected set of baseline values or reference standards). A baseline value or reference standard may be a quantity of cell types measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a quantity of cell types measured in one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples (such as a tissue sample or a blood sample, e.g., a whole blood sample, a buffy coat sample, a leukapheresis sample, or a PBMC sample) may be collected from a subject at two or more timepoints, to assess changes in cell types (such as changes in quantities of cell types) between the two or more timepoints. In some embodiments, a sample collected at a first time point is a tissue sample or a blood sample, and a sample collected at a subsequent time point (such as a second time point) is a blood sample. In some embodiments, a sample collected at a first time point is a tissue sample and a sample collected at a subsequent time point (such as a second time point) is a blood sample. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample such as at least one tissue sample and/or at least one blood sample, e.g., a whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

As disclosed herein, methods are provided for monitoring one or more aspects of a condition in a subject over time, such as but not limited to, a subject's response to receiving a treatment for a condition (such as a response to a chemotherapeutic or immunotherapeutic). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment, or the course or stage of a condition (such as a cancer) in the subject is being monitored over time, cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples (such as one or more tissue, whole blood, buffy coat, leukapheresis, or PBMC samples) is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples (such as one or more tissue samples or blood samples, e.g., or one or more buffy coat samples, whole blood samples, leukapheresis samples, or PBMC samples) are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

### 4. Therapies and Related Administration

In certain embodiments, the methods disclosed herein relate to identifying and administering customized therapies, such as customized therapies to patients. In some embodiments, determination of the levels of particular immune cell types, including rare immune cell types, facilitates selection of appropriate treatment. In some embodiments, the patient or subject has a given disease, disorder or condition, e.g., any of the cancers or other conditions described elsewhere herein. Essentially any cancer therapy (e.g., surgical therapy, radiation therapy, chemotherapy, immunotherapy, and/or the like) may be included as part of these methods. In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, therapy is customized based on the status of a nucleic acid variant as being of somatic or germline origin. In some embodiments, essentially any cancer therapy (e.g., surgical therapy, radiation therapy, chemotherapy, immunotherapy, and/or the like) may be included as part of these methods. Customized therapies can include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g., antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Table 6 provides an exemplary list of drugs used to treat cancers with mutations observed in target genes associated with certain cancer types. In certain embodiments, the subject has a cancer of a type listed in Table 6 including a mutation in one or more target genes listed in Table 6 for that cancer type, and the therapy administered to the subject comprises the drug listed in Table 6 for that cancer type and mutation.

TABLE 6
Exemplary drugs

| Cancer type | Drug | Target genes |
|---|---|---|
| Breast | abemaciclib (Verzenio) | CDK4, CDK6 |
| prostate | abiraterone acetate (Zytiga) | CYP17A1 |
| leukemia | acalabrutinib (Calquence) | BTK |
| lymphoma | acalabrutinib (Calquence) | BTK |
| lung | adagrasib (Krazati) | KRAS G12C |
| Breast | ado-trastuzumab emtansine | Tubulin, Her2 |
| | (Kadcyla) | |
| lung | afatinib dimaleate (Gilotrif) | EGFR + EGFR exon 21 L858R |
| | | mutation + EGFR-Ex19del + |
| | | HER2 + HER4 |
| lung | alectinib (Alecensa) | ALK, RET |
| leukemia | alemtuzumab (Campath) | CD52 |
| skin | alitretinoin (Panretin) | RARα + RARβ2 + RARγ |
| soft tissue | alitretinoin (Panretin) | RARα + RARβ2 + RARγ |
| sarcoma | | |
| Breast | alpelisib (Piqray) | PI3Kα |
| lung | amivantamab-vmjw | EGFR + c-MET |
| | (Rybrevant) | |
| Breast | anastrozole (Arimidex) | Aromatase |
| prostate | apalutamide (Erleada) | AR |
| leukemia | asciminib hydrochloride | Bcr-Abl |
| | (Scemblix) | |
| bladder | atezolizumab (Tecentriq) | PDL1 |
| liver and bile | atezolizumab (Tecentriq) | PDL1 |
| duct | | |
| lung | atezolizumab (Tecentriq) | PDL1 |
| skin | atezolizumab (Tecentriq) | PDL1 |
| soft tissue | atezolizumab (Tecentriq) | PDL1 |
| sarcoma | | |
| Gastrointestinal | avapritinib (Ayvakit) | PDGFRα + c-Kit |
| systemic | avapritinib (Ayvakit) | PDGFRα + c-Kit |
| mastocytosis | | |
| bladder | avelumab (Bavencio) | PDL1 |
| endocrine | avelumab (Bavencio) | PDL1 |
| and | | |
| neuroendocri | | |
| ne tumors | | |
| kidney | avelumab (Bavencio) | PDL1 |
| skin | avelumab (Bavencio) | PDL1 |
| lymphoma | axicabtagene ciloleucel | CD19 |
| | (Yescarta) | |
| kidney | axitinib (Inlyta) | VEGFR1 + VEGFR2 + VEGFR3 |
| lymphoma | belinostat (Beleodaq) | HDAC |
| Brain | belzutifan (Welireg) | HIF-2A |
| kidney | belzutifan (Welireg) | HIF-2A |
| pancreatic | belzutifan (Welireg) | HIF-2A |
| ovarian | bevacizumab (Avastin) | VEGF-A |
| epithelial, | | |
| fallopian | | |
| tube, and | | |
| primary | | |
| peritoneal | | |
| Brain | bevacizumab (Avastin) | VEGF-A |
| Cervical | bevacizumab (Avastin) | VEGF-A |
| colorectal | bevacizumab (Avastin) | VEGF-A |
| kidney | bevacizumab (Avastin) | VEGF-A |
| liver and bile | bevacizumab (Avastin) | VEGF-A |
| duct | | |
| lung | bevacizumab (Avastin) | VEGF-A |
| lymphoma | bexarotene (Targretin) | RXRs |
| skin | binimetinib (Mektovi) | MEK1, MEK2 |
| leukemia | blinatumomab (Blincyto) | CD19, CD3 |
| lymphoma | bortezomib (Velcade) | Proteasome |
| multiple | bortezomib (Velcade) | Proteasome |
| myeloma | | |
| leukemia | bosutinib (Bosulif) | BCR-ABL, SRC |
| lymphoma | brentuximab vedotin | CD130, Tubulin |
| | (Adcetris) | |
| leukemia | brexucabtagene autoleucel | CD19 |
| | (Tecartus) | |
| lymphoma | brexucabtagene autoleucel | CD19 |
| | (Tecartus) | |
| lung | brigatinib (Alunbrig) | ALK, EGFR, FLT3, IGF-1R, |
| | | ROS1 |
| prostate | cabazitaxel (Jevtana) | Tubulin |
| kidney | cabozantinib-s-malate | AXL + RET + ROS1 + TYRO3 + |
| | (Cabometyx) | Tie-2 + TrkB + VEGFR1 + |
| | | VEGFR2 + VEGFR3 + c-Kit + c- |
| | | Met |
| liver and bile | cabozantinib-s-malate | AXL+ RET + ROS1 + TYRO3 + |
| duct | (Cabometyx) | Tie-2 + TrkB + VEGFR1 + |
| | | VEGFR2 + VEGFR3 + c-Kit + c- |
| | | Met |
| thyroid | cabozantinib-s-malate | AXL + RET + ROS1 + TYRO3 + |
| | (Cometriq) | Tie-2 + TrkB + VEGFR1 + |
| | | VEGFR2 + VEGFR3 + c-Kit + c- |
| | | Met |
| lung | capmatinib hydrochloride | c-Met |
| | (Tabrecta) | |
| multiple | carfilzomib (Kyprolis) | protoasome |
| myeloma | | |
| lung | cemiplimab-rwlc (Libtayo) | PD-1 |
| skin | cemiplimab-rwlc (Libtayo) | PD-1 |
| lung | ceritinib (Zykadia) | ALK + IGF-1R + INSR + ROS1 |
| colorectal | cetuximab (Erbitux) | EGFR |
| multiple | ciltacabtagene autoleucel | BCMA |
| myeloma | (Carvykti) | |
| skin | cobimetinib fumarate | MEK1, MEK2 |
| | (Cotellic) | |
| lymphoma | copanlisib hydrochloride | PI3Kα, PI3Kδ |
| | (Aliqopa) | |
| lung | crizotinib (Xalkori) | MTH1 |
| lymphoma | crizotinib (Xalkori) | MTH1 |
| Myofibroblastic | crizotinib (Xalkori) | MTH1 |
| Brain | dabrafenib (Tafinlar) | BRAF, CRAF |
| lung | dabrafenib mesylate | BRAF, CRAF |
| | (Tafinlar) | |
| skin | dabrafenib mesylate | BRAF, CRAF |
| | (Tafinlar) | |
| solid tumors | dabrafenib mesylate | BRAF, CRAF |
| anywhere in | (Tafinlar) | |
| the body | | |
| thyroid | dabrafenib mesylate | BRAF, CRAF |
| | (Tafinlar) | |
| lung | dacomitinib (Vizimpro) | EGFR + EGFR exon 21 L858R |
| | | mutation + EGFR-Ex19del + |
| | | HER2 + HER4 |
| multiple | daratumumab (Darzalex) | CD38 + Hyaluronic acid |
| myeloma | | |
| multiple | daratumumab and | CD38 + Hyaluronic acid |
| myeloma | hyaluronidase-fihj | |
| | (Darzalex Faspro) | |
| prostate | darolutamide (Nubeqa) | AR |
| leukemia | dasatinib (Sprycel) | Bcr-Abl + EphA2 + FYN + LCK + |
| | | PDGFRβ + Protein-tyrosine |
| | | kinases + SRC + YES1 + c-Kit |
| neuroblastoma | dinutuximab (Unituxin) | GD2 |
| endometrial | dostarlimab-gxly (Jemperli) | PD-1 |
| solid tumors | dostarlimab-gxly (Jemperli) | PD-1 |
| anywhere in | | |
| the body | | |
| liver and bile | durvalumab (Imfinzi) | PDL1 |
| duct | | |
| lung | durvalumab (Imfinzi) | PDL1 |
| leukemia | duvelisib (Copiktra) | PI3Kγ + PI3Kδ |
| Breast | elacestrant dihydrochloride | Erα/ESR1 |
| | (Orserdu) | |
| multiple | elotuzumab (Empliciti) | SLAMF7 |
| myeloma | | |
| leukemia | enasidenib mesylate (Idhifa) | IDH2 |
| colorectal | encorafenib (Braftovi) | BRAF, BRAF V600E |
| skin | encorafenib (Braftovi) | BRAF V600E, V600K mutation |
| bladder | enfortumab vedotin-ejfv | Nectin-4, Tubulins |
| | (Padcev) | |
| lung | entrectinib (Rozlytrek) | ALK + ROS1 + TRKA + TrkB + |
| | | TrkC |
| solid tumors | entrectinib (Rozlytrek) | ALK + ROS1 + TRKA + TrkB + |
| anywhere in | | TrkC |
| the body | | |
| prostate | enzalutamide (Xtandi) | AR |
| bladder | erdafitinib (Balversa) | FGFR1, FGFR2, FGFR3, FGFR4 |
| lung | erlotinib hydrochloride | EGFR antagonists, EGFR exon |
| | (Tarceva) | 21 L858R mutation inhibitors, |
| | | EGFR-Ex19del inhibitors |
| pancreatic | erlotinib hydrochloride | EGFR + EGFR exon 21 L858R |
| | (Tarceva) | mutation + EGFR-Ex19del |
| Breast | everolimus (Afinitor) | mTORC1, mTORC2 |
| kidney | everolimus (Afinitor) | mTORC1, mTORC2 |
| pancreatic | everolimus (Afinitor) | mTORC1, mTORC2 |
| Breast | exemestane (Aromasin) | Aromatase |
| Breast | fam-trastuzumab | HER2, TOP1 |
| | deruxtecan-nxki (Enhertu) | |
| Gastric | fam-trastuzumab | HER2, TOP1 |
| | deruxtecan-nxki (Enhertu) | |
| lung | fam-trastuzumab | HER2, TOP1 |
| | deruxtecan-nxki (Enhertu) | |
| myelodysplas | fedratinib hydrochloride | JAK2, FLT3 |
| tic and | (Inrebic) | |
| myeloprolifer | | |
| ative | | |
| disorders | | |
| Breast | fulvestrant (Faslodex) | ER |
| liver and bile | futibatinib (Lytgobi) | FGFRs |
| duct | | |
| lung | gefitinib (Iressa) | EGFR + EGFR exon 21 L858R |
| | | mutation + EGFR-Ex19del |
| leukemia | gemtuzumab ozogamicin | CD33, DNA |
| | (MYLOTARG) | |
| leukemia | gilteritinib fumarate | AXL + FLT3 |
| | (Xospata) | |
| leukemia | glasdegib | SMO |
| | maleate (Daurismo) | |
| lymphoma | ibritumomab tiuxetan | CD20 |
| | (Zevalin) | |
| leukemia | ibrutinib (Imbruvica) | BTK |
| lymphoma | ibrutinib (Imbruvica) | BTK |
| multiple | idecabtagene vicleucel | BCMA |
| myeloma | (Abecma) | |
| leukemia | idelalisib (Zydelig) | PI3Kδ |
| dermatofibro | imatinib mesylate | BCR-ABL, PDGFR, C-Kit |
| sarcoma | (Gleevec) | |
| protuberans | | |
| Gastrointesti | imatinib mesylate | BCR-ABL, PDGFR, C-Kit |
| nal | (Gleevec) | |
| leukemia | imatinib mesylate | BCR-ABL, PDGFR, C-Kit |
| | (Gleevec) | |
| myelodysplas | imatinib mesylate | BCR-ABL, PDGFR, C-Kit |
| tic and | (Gleevec) | |
| myeloprolifer | | |
| ative | | |
| disorders | | |
| systemic | imatinib mesylate | BCR-ABL, PDGFR, C-Kit |
| mastocytosis | (Gleevec) | |
| liver and bile | infigratinib phosphate | FGFR1 + FGFR2 + FGFR3 + |
| duct | (Truseltiq) | FGFR4 |
| leukemia | inotuzumab ozogamicin | CD22 + DNA |
| | (Besponsa) | |
| endocrine | iobenguane I 131 | NET |
| and | (Azedra) | |
| neuroendocrine | | |
| tumors | | |
| colorectal | ipilimumab (Yervoy) | CTLA4 |
| esophageal | ipilimumab (Yervoy) | CTLA4 |
| kidney | ipilimumab (Yervoy) | CTLA4 |
| liver and bile | ipilimumab (Yervoy) | CTLA4 |
| duct | | |
| lung | ipilimumab (Yervoy) | CTLA4 |
| malignant | ipilimumab (Yervoy) | CTLA4 |
| mesotheliom | | |
| a | | |
| skin | ipilimumab (Yervoy) | CTLA4 |
| multiple | isatuximab-irfc (Sarclisa) | CD38 |
| myeloma | | |
| leukemia | ivosidenib (Tibsovo) | IDH1 |
| multiple | ixazomib citrate (Ninlaro) | proteasome |
| myeloma | | |
| endocrine | lanreotide acetate | SSTR |
| and | (Somatuline Depot) | |
| neuroendocri | | |
| ne tumors | | |
| Breast | lapatinib ditosylate | EGFR |
| | (Tykerb) | |
| solid tumors | larotrectinib sulfate | TRKA + TrkB + TrkC |
| anywhere in | (Vitrakvi) | |
| the body | | |
| endometrial | lenvatinib mesylate | FGFR1 + FGFR2 + FGFR3 + |
| | (Lenvima) | FGFR4 + PDGFRα + RET + |
| | | VEGFR1 + VEGFR2 + VEGFR3 + |
| | | c-Kit |
| kidney | lenvatinib mesylate | FGFR1 + FGFR2 + FGFR3 + |
| | (Lenvima) | FGFR4 + PDGFRα + RET + |
| | | VEGFR1 + VEGFR2 + VEGFR3 + |
| | | c-Kit |
| liver and bile | lenvatinib mesylate | FGFR1 + FGFR2 + FGFR3 + |
| duct | (Lenvima) | FGFR4 + PDGFRα + RET + |
| | | VEGFR1 + VEGFR2 + VEGFR3 + |
| | | c-Kit |
| thyroid | lenvatinib mesylate | FGFR1 + FGFR2 + FGFR3 + |
| | (Lenvima) | FGFR4 + PDGFRα + RET + |
| | | VEGFR1 + VEGFR2 + VEGFR3 + |
| | | c-Kit |
| Breast | letrozole | CDK4/ER |
| | (Femara)/Ribociclib | |
| | Succinate | |
| lymphoma | lisocabtagene maraleucel | CD19 |
| | (Breyanzi) | |
| lymphoma | loncastuximab tesirine-lpyl | CD19 + DNA |
| | (Zynlonta) | |
| lung | lorlatinib (Lorbrena) | ALK + ROS1 |
| prostate | lutetium Lu 177 vipivotide | PSMA |
| | tetraxetan (Pluvicto) | |
| endocrine | lutetium Lu 177-dotatate | SSTR2 |
| and | (Lutathera) | |
| neuroendocri | | |
| ne tumors | | |
| Breast | margetuximab-cmkb | HER2 |
| | (Margenza) | |
| leukemia | midostaurin (Rydapt) | FLT3 + PDGFR + PKC + Syk + |
| | | VEGFR2 + c-Kit |
| systemic | midostaurin (Rydapt) | FLT3 + PDGFR + PKC + Syk + |
| mastocytosis | | VEGFR2 + c-Kit |
| ovarian | mirvetuximab soravtansine- | FOLR1 + Tubulin |
| epithelial, | gynx (Elahere) | |
| fallopian | | |
| tube, and | | |
| primary | | |
| peritoneal | | |
| lung | mobocertinib succinate | EGFR exon 20 + HER2 exon 20 |
| | (Exkivity) | |
| lymphoma | mogamulizumab-kpkc | CCR4 |
| | (Poteligeo) | |
| lymphoma | mosunetuzumab-axgb | CD20, CD3 |
| | (Lunsumio) | |
| leukemia | moxetumomab pasudotox- | CD22 |
| | tdfk (Lumoxiti) | |
| neuroblastom | naxitamab-gqgk (Danyelza) | GD2 |
| a | | |
| lung | necitumumab (Portrazza) | EGFR |
| Breast | neratinib maleate (Nerlynx) | EGFR, HER2, HER4 |
| leukemia | nilotinib (Tasigna) | Bcr-Abl + CSF-1R + DDR1 + |
| | | PDGFR + c-Kit |
| ovarian | niraparib tosylate | PARP1 + PARP2 |
| epithelial, | monohydrate (Zejula) | |
| fallopian | | |
| tube, and | | |
| primary | | |
| peritoneal | | |
| bladder | nivolumab (Opdivo) | PD-1 |
| colorectal | nivolumab (Opdivo) | PD-1 |
| esophageal | nivolumab (Opdivo) | PD-1 |
| kidney | nivolumab (Opdivo) | PD-1 |
| liver and bile | nivolumab (Opdivo) | PD-1 |
| duct | | |
| lung | nivolumab (Opdivo) | PD-1 |
| lymphoma | nivolumab (Opdivo) | PD-1 |
| malignant | nivolumab (Opdivo) | PD-1 |
| mesotheliom | | |
| a | | |
| skin | nivolumab (Opdivo) | PD-1 |
| stomach | nivolumab (Opdivo) | PD-1 |
| (gastric) | | |
| skin | nivolumab and relatlimab- | LAG3 + PD-1 |
| | rmbw (Opdualag) | |
| leukemia | obinutuzumab (Gazyva) | CD20 |
| lymphoma | obinutuzumab (Gazyva) | CD20 |
| leukemia | ofatumumab (Arzerra) | CD20 |
| ovarian | olaparib (Lynparza) | PARP1, PARP2, PARP3 |
| epithelial, | | |
| fallopian | | |
| tube, and | | |
| primary | | |
| peritoneal | | |
| Breast | olaparib (Lynparza) | PARP1, PARP2, PARP3 |
| pancreatic | olaparib (Lynparza) | PARP1, PARP2, PARP3 |
| prostate | olaparib (Lynparza) | PARP1, PARP2, PARP3 |
| leukemia | olutasidenib (Rezlidhia) | IDH1 |
| lung | osimertinib mesylate | EGFR + EGFR T790M + EGFR |
| | (Tagrisso) | exon 21 L858R mutation + |
| | | EGFR-Ex19del |
| myelodysplas | pacritinib citrate (Vonjo) | CSF-1R + FLT3 + IRAK1 + |
| tic and | | JAK2 |
| myeloprolifer | | |
| ative | | |
| disorders | | |
| Breast | palbociclib (Ibrance) | CDK4, CDK6 |
| colorectal | panitumumab (Vectibix) | EGFR |
| kidney | pazopanib | FGFR1 + FGFR3 + Flt3L + ITK + |
| | hydrochloride (Votrient) | LCK + PDGFRα + PDGFRβ + |
| | | VEGFR1 + VEGFR2 + VEGFR3 + |
| | | c-Kit |
| soft tissue | pazopanib hydrochloride | FGFR1 + FGFR3 + Flt3L + ITK + |
| sarcoma | (Votrient) | LCK + PDGFRα + PDGFRβ + |
| | | VEGFR1 + VEGFR2 + VEGFR3 + |
| | | c-Kit |
| Breast | pembrolizumab (Keytruda) | PD-1 |
| Cervical | pembrolizumab (Keytruda) | PD-1 |
| colorectal | pembrolizumab (Keytruda) | PD-1 |
| endometrial | pembrolizumab (Keytruda) | PD-1 |
| esophageal | pembrolizumab (Keytruda) | PD-1 |
| kidney | pembrolizumab (Keytruda) | PD-1 |
| liver and bile | pembrolizumab (Keytruda) | PD-1 |
| duct | | |
| lung | pembrolizumab (Keytruda) | PD-1 |
| lymphoma | pembrolizumab (Keytruda) | PD-1 |
| skin | pembrolizumab (Keytruda) | PD-1 |
| solid tumors | pembrolizumab (Keytruda) | PD-1 |
| anywhere in | | |
| the body | | |
| stomach | pembrolizumab (Keytruda) | PD-1 |
| (gastric) | | |
| leukemia | pemigatinib (Pemazyre) | FGFR1, FGFR2, FGFR3, FGFR4 |
| liver and bile | pemigatinib (Pemazyre) | FGFR1, FGFR2, FGFR3, FGFR4 |
| duct | | |
| lymphoma | pemigatinib (Pemazyre) | FGFR1, FGFR2, FGFR3, FGFR4 |
| myelodysplas | pemigatinib (Pemazyre) | FGFR1, FGFR2, FGFR3, FGFR4 |
| tic and | | |
| myeloprolifer | | |
| ative | | |
| disorders | | |
| Breast | pertuzumab (Perjeta) | HER2 |
| Breast | pertuzumab, trastuzumab, | HER2, Hyaluronic acid |
| | and hyaluronidase-zzxf | |
| | (Phesgo) | |
| giant cell | pexidartinib hydrochloride | CSF-1R + FLT3 + c-Kit |
| tumor | (Turalio) | |
| lymphoma | pirtobrutinib (Jaypirca) | BTK C481S |
| lymphoma | polatuzumab vedotin-piiq | CD79B + Tubulin |
| | (Polivy) | |
| leukemia | ponatinib hydrochloride | Bcr-Abl + Protein-tyrosine |
| | (Iclusig) | kinases |
| lymphoma | pralatrexate (Folotyn) | Bcr-Abl + Protein-tyrosine |
| | | kinases |
| lung | pralsetinib (Gavreto) | RET |
| thyroid | pralsetinib (Gavreto) | RET |
| prostate | radium 223 dichloride | DNA |
| | (Xofigo) | |
| colorectal | ramucirumab (Cyramza) | VEGFR2 |
| Gastric | ramucirumab (Cyramza) | VEGFR2 |
| liver and bile | ramucirumab (Cyramza) | VEGFR2 |
| duct | | |
| lung | ramucirumab (Cyramza) | VEGFR2 |
| stomach | ramucirumab (Cyramza) | VEGFR2 |
| (gastric) | | |
| colorectal | regorafenib (Stivarga) | Abl family, BRAF, BRAF |
| | | V600E, CRAF, CSF-1R, DDR2, |
| | | EphA2, FGFR1, FGFR2, FRK, |
| | | MAPK11, PDGFRa, PDGFRb, |
| | | RET, TRKA, Tie-2, VEGFR1, |
| | | VEGFR2, VEGFR3, c-kit |
| Gastrointesti | regorafenib (Stivarga) | Abl family, BRAF, BRAF |
| nal | | V600E, CRAF, CSF-1R, DDR2, |
| | | EphA2, FGFR1, FGFR2, FRK, |
| | | MAPK11, PDGFRa, PDGFRb, |
| | | RET, TRKA, Tie-2, VEGFR1, |
| | | VEGFR2, VEGFR3, c-kit |
| liver and bile | regorafenib (Stivarga) | Abl family, BRAF, BRAF |
| duct | | V600E, CRAF, CSF-1R, DDR2, |
| | | EphA2, FGFR1, FGFR2, FRK, |
| | | MAPK11, PDGFRa, PDGFRb, |
| | | RET, TRKA, Tie-2, VEGFR1, |
| | | VEGFR2, VEGFR3, c-kit |
| skin | retifanlimab-dlwr (Zynyz) | PD-1 |
| Breast | ribociclib (Kisqali) | CDK4, CDK6 |
| gastrointestin | ripretinib (Qinlock) | EGFR + PDGFRα + c-Kit |
| al | | |
| leukemia | rituximab (Rituxan) | CD20 + Hyaluronic acid |
| lymphoma | rituximab (Rituxan) | CD20 + Hyaluronic acid |
| leukemia | rituximab and hyaluronidase | CD20 + Hyaluronic acid |
| | human (Rituxan Hycela) | |
| lymphoma | rituximab and hyaluronidase | CD20 + Hyaluronic acid |
| | human (Rituxan Hycela) | |
| lymphoma | romidepsin (Istodax) | HDAC |
| ovarian | rucaparib | PARP1 + PARP2 + PARP3 |
| epithelial, | camsylate (Rubraca) | |
| fallopian | | |
| tube, and | | |
| primary | | |
| peritoneal | | |
| prostate | rucaparib camsylate | PARP1 + PARP2 + PARP3 |
| | (Rubraca) | |
| myelodysplas | ruxolitinib phosphate | JAK1 + JAK2 |
| tic and | (Jakafi) | |
| myeloprolifer | | |
| ative | | |
| disorders | | |
| Breast | sacituzumab govitecan-hziy | TOP1 |
| | (Trodelvy) | |
| lymphoma | selinexor (Xpovio) | XPO1 |
| multiple | selinexor (Xpovio) | XPO1 |
| myeloma | | |
| lung | selpercatinib (Retevmo) | RET |
| solid tumors | selpercatinib (Retevmo) | RET |
| anywhere in | | |
| the body | | |
| thyroid | selpercatinib (Retevmo) | RET |
| plexiform | selumetinib sulfate | MEK1 + MEK2 |
| neurofibroma | (Koselugo) | |
| soft tissue | sirolimus protein-bound | MUT + mTOR |
| sarcoma | particles (Fyarro) | |
| skin | sonidegib (Odomzo) | SMO |
| kidney | sorafenib tosylate (Nexavar) | BRAF inhibitors, CRAF |
| | | inhibitors, FLT3 + PDGFRβ + |
| | | RET + VEGFR1 + VEGFR2 + |
| | | VEGFR3 + c-Kit |
| liver and bile | sorafenib tosylate (Nexavar) | BRAF + CRAF + FLT3 + |
| duct | | PDGFRβ + RET + VEGFR1 + |
| | | VEGFR2 + VEGFR3 + c-Kit |
| thyroid | sorafenib tosylate (Nexavar) | BRAF + CRAF + FLT3 + |
| | | PDGFRβ + RET + VEGFR1 + |
| | | VEGFR2 + VEGFR3 + c-Kit |
| lung | sotorasib (Lumakras) | KRAS G12C |
| Gastrointesti | sunitinib malate (Sutent) | PDGFR + RTK + VEGFR |
| nal | | |
| kidney | sunitinib malate (Sutent) | PDGFR + RTK + VEGFR |
| pancreatic | sunitinib malate (Sutent) | PDGFR + RTK + VEGFR |
| lymphoma | tafasitamab-cxix (Monjuvi) | CD19 |
| leukemia | tagraxofusp-erzs (Elzonris) | CD123 |
| Breast | talazoparib tosylate | PARP1, PARP2 |
| | (Talzenna) | |
| Breast | tamoxifen citrate (Soltamox) | ER |
| lymphoma | tazemetostat hydrobromide | EZH2 |
| | (Tazverik) | |
| soft tissue | tazemetostat hydrobromide | EZH2 |
| sarcoma | (Tazverik) | |
| skin | tebentafusp-tebn (Kimmtrak) | CD3 + gp100 |
| multiple | teclistamab-cqyv (Tecvayli) | CD3 + gp100 |
| myeloma | | |
| kidney | temsirolimus (Torisel) | CD3 + gp100 |
| lung | tepotinib hydrochloride | MET |
| | (Tepmetko) | |
| leukemia | tisagenlecleucel (Kymriah) | CD19 |
| lymphoma | tisagenlecleucel (Kymriah) | CD19 |
| Cervical | tisotumab vedotin-tftv | Tubulin, tissue factor |
| | (Tivdak) | |
| kidney | tivozanib hydrochloride | (VEGFR)-1, VEGFR-2 and |
| | (Fotivda) | VEGFR-3, c-kit, and PDGFR β |
| Breast | toremifene (Fareston) | ER |
| Brain | trametinib (Mekinist) | MEK1 and MEK2 |
| lung | trametinib dimethyl | MEK1 and MEK2 |
| | sulfoxide (Mekinist) | |
| skin | trametinib dimethyl | MEK1 and MEK2 |
| | sulfoxide (Mekinist) | |
| solid tumors | trametinib dimethyl | MEK1 and MEK2 |
| anywhere in | sulfoxide (Mekinist) | |
| the body | | |
| thyroid | trametinib dimethyl | MEK1 and MEK2 |
| | sulfoxide (Mekinist) | |
| Breast | trastuzumab (Herceptin) | HER2 |
| esophageal | trastuzumab (Herceptin) | HER2 |
| stomach | trastuzumab (Herceptin) | HER2 |
| (gastric) | | |
| liver and bile | tremelimumab-actl (Imjudo) | CTLA-4, CD80, and CD86 |
| duct | | |
| lung | tremelimumab-actl (Imjudo) | CTLA-4, CD80, and CD86 |
| Breast | tucatinib (Tukysa) | HER2 |
| colorectal | tucatinib (Tukysa) | HER2 |
| thyroid | vandetanib (Caprelsa) | VEGFR-2, EGFR, RET |
| skin | vemurafenib (Zelboraf) | BRAF, BRAF V600E, CRAF, |
| | | ARAF, SRMS, ACKI, MAP4K5, |
| | | FGR |
| leukemia | venetoclax (Venclexta) | BCL-2 |
| lymphoma | venetoclax (Venclexta) | BCL-2 |
| lymphoma | vorinostat (Zolinza) | HDAC1, HDAC2 and HDAC3 |
| | | (Class I) and HDAC6 (Class II) |
| leukemia | zanubrutinib (Brukinsa) | BTK |
| lymphoma | zanubrutinib (Brukinsa) | BTK |
| colorectal | ziv-aflibercept (Zaltrap) | PGF, VEGF-A |

In some embodiments, the methods described herein can be used to treat patients by (i) detecting one or more somatic mutations and/or cancer-related epigenetic signatures in the one or more target genes listed in Table 6; and (ii) administering the corresponding one or more drugs listed in Table 6. In some embodiments, these therapies may be used alone or in combination with other therapies to treat a disease.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

In some embodiments, therapy is customized based on the status of a nucleic acid variant as being of somatic or germline origin. In some embodiments, determination of the levels of particular cell types, e.g., immune cell types, including rare immune cell types, facilitates selection of appropriate treatment.

The present methods can be used to diagnose the presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The methods according to the present disclosure can also be useful in predicting a subject's response to a particular treatment option. Successful treatment options may increase the amount of copy number variation, rare mutations, and/or cancer-related epigenetic signatures (such as hypermethylated regions or hypomethylated regions) detected in a subject's blood (such as in DNA isolated from a buffy coat sample or any other sample comprising cells, such as a blood sample (e.g., a whole blood sample, a buffy coat sample, a leukapheresis sample, or a PBMC sample) from the subject) if the treatment is successful as more cancer cells may die and shed DNA, or if a successful treatment results in an increase or decrease in the quantity of a specific immune cell type in the blood and an unsuccessful treatment results in no change. In other examples, this may not occur. In another example, certain treatment options may be correlated with genetic profiles of cancers over time. This correlation may be useful in selecting a therapy for a subject. In some embodiments, determination of the metastasis site facilitates selection of appropriate treatment.

Thus, in some embodiments, quantities of each of one or more of a particular genetic and/or epigenetic signature (e.g., quantities of fusions, indels, SNPs, CNVs, and/or rare mutations, and/or cancer-related epigenetic signatures (such as specific (e.g., DMRs) or global hypermethylated or hypomethylated regions, and/or fragmentation variable regions)) in DNA from a subject's blood (such as in DNA (e.g., cfDNA) isolated from a blood sample (e.g., a whole blood sample) from the subject)) are determined based on sequencing and analysis. In some embodiments, quantities of each of a plurality of cell types, such as immune cell types, are determined based on sequencing and analysis (such as determination of epigenetic and/or genomic signatures) of DNA isolated from at least one sample comprising cells (such as blood sample (e.g., a whole blood sample, a buffy coat sample, a leukapheresis sample, or a PBMC sample) from a subject. The plurality of immune cell types can include, but is not limited to, macrophages (including M1 macrophages and M2 macrophages), activated B cells (including regulatory B cells, memory B cells and plasma cells); T cell subsets, such as central memory T cells, naïve-like T cells, and activated T cells (including cytotoxic T cells, regulatory T cells (Tregs), CD4 effector memory T cells, CD4 central memory T cells, CD8 effector memory T cells, and CD8 central memory T cells); immature myeloid cells (including myeloid-derived suppressor cells (MDSCs), low-density neutrophils, immature neutrophils, and immature granulocytes); and natural killer (NK) cells. As disclosed herein, differences in levels and/or presence of particular genetic and/or epigenetic signatures in DNA isolated from blood samples from a subject can be used to quantify cell types, such as immune cell types, within the sample. Thus, a comparison of one or more genetic and/or epigenetic signatures in DNA isolated from blood samples collected from a subject at two or more time points can be used to monitor changes in the one or more signatures and/or the one or more cell type quantities in the subject under different conditions (such as prior to and after a treatment), or over time (e.g., as part of a preventative health monitoring program).

In some embodiments, therapy is customized based on the status of a detected nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

The disclosed methods can include evaluating (such as quantifying) and/or interpreting at least one cell material released from a potential metastasis site (such as at least one cell material in a sample from a subject) and/or cell types that contribute to DNA, such as cfDNA, in one or more samples collected from a subject at one or more timepoints in comparison to a selected baseline value or reference standard (or a selected set of baseline values or reference standards). A baseline value or reference standard may be a presence or level of at least one cell material and/or a quantity of cell types measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a presence or level of at least one cell material and/or a quantity of cell types measured with respect to one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

The disclosed methods can include evaluating (such as quantifying) and/or interpreting one or more genetic and/or epigenetic signatures, and/or one or more cell types (such as one or more immune cell types), present in one or more samples (e.g., in DNA, such as cfDNA, from a blood sample(e.g., a whole blood sample, a buffy coat sample, a leukapheresis sample, or a PBMC sample)) collected from a subject at one or more timepoints in comparison to a selected baseline value or reference standard (or a selected set of baseline values or reference standards). A baseline value or reference standard may be a quantity of copy number variation, rare mutations, cancer-related epigenetic signatures (such as hypermethylated regions or hypomethylated regions), and/or cell types measured in one or more samples (such as an average quantity or range of quantities of such signatures present in at least two samples) collected from the subject at one or more time points, such as prior to receiving a treatment, prior to diagnosis of a condition (such as a cancer), or as part of a preventative health monitoring program. A baseline value or reference standard may be a quantity of, e.g., copy number variation, rare mutations, cancer-related epigenetic signatures (such as hypermethylated regions or hypomethylated regions), and/or cell types measured in one or more samples (such as an average quantity or range of quantities of such signatures and/or cell types present in at least two samples) collected at one or more timepoints from one or more subjects that do not have the condition (such as a healthy subject that does not have a cancer), one or more subjects that responded favorably to the treatment, or one or more subjects that have not received the treatment.

In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the genetic and/or epigenetic signature quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples comprising cells (such as a buffy coat sample or any other sample comprising cells, such as a blood sample (e.g., a whole blood sample, a leukapheresis sample, or a PBMC sample) may be collected from a subject at two or more timepoints, to assess changes in cell types (such as changes in quantities of cell types) between the two timepoints. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample (such as at least one whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

In some embodiments, methods are provided for monitoring a response (such as a change in disease state, such as a presence or absence of a metastasis in a subject, such as measured by assessing a presence or level of at least one cell material released from a potential metastasis site in a sample from the subject) of a subject to a treatment (such as a chemotherapy or an immunotherapy). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment or the course or stage of a condition (such as a cancer) in the subject is being monitored over time, genetic and/or epigenetic signatures, and/or cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples (such as one or more whole blood, buffy coat, leukapheresis, or PBMC samples) are collected from a subject at least once per week, such as on 1-4 days, 1-2 days, or on 1, 2, 3, 4, 5, 6, or 7 days per week. In certain embodiments, one or more samples are collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

Therapeutic options for treating specific genetic-based diseases, disorders, or conditions, other than cancer, are generally well-known to those of ordinary skill in the art and will be apparent given the particular disease, disorder, or condition under consideration.

### H. Table of Sequences

The following table shows exemplary sequences provided herein.

| SEQ ID NO | |
|---|---|
| and | |
| description | Sequence |
| SEQ ID NO: 1 | MNSNKDKIKVIKVFEAFAGIGSQFKALKNIARSKNWEIQHSGMVEWFVDAIVSYVAIHSKN |
| A variant of | FNPKIERLDRDILSISNDSKMPISEYGIKKINNTIKASYLNYAKKHFNNLFDIKKVNKDNF |
| M. MpeI | PKNIDIFTYSFPCQDLSVQGLQKGIDKELNTRSGLLWEIERILEEIKNSFSKEEMPKYLLM |
| | ENVKNLLSHKNKKNYNTWLKQLEKFGYKSKTYLLNSKNFDNCQNRERVFCLSIRDDYLEKT |
| | GFKFKELEKVKNPPKKIKDILVDSSNYKYLNLNKYETTTFRETKSNIISRPLKNYTTFNSE |
| | NYVYNINGIGPTLTASGANSRIKIETQQGVRYLTPLECFKYMQFDVNDFKKVQSTNLISEN |
| | KMIYIAGKSIPVKILEAIFNTLEFVNNEELE |
| SEQ ID NO: 2 | MNSNKDKIKVIKVFEAFAGIGSQFKALKNIARSKNWEIQHSGMVEWFVDAIVSYVAIHSKN |
| A variant of | FNPKIERLDRDILSISNDSKMPISEYGIKKINNTIKASYLNYAKKHFNNLFDIKKVNKDNF |
| M. MpeI | PKNIDIFTYSFPCQDLSVQGLQKGIDKELNTRSGLLWEIERILEEIKNSFSKEEMPKYLLM |
| | ENVKNLLSHKNKKNYNTWLKQLEKFGYKSKTYLLNSKNFDNCQNRERVFCLSIRDDYLEKT |
| | GFKFKELEKVKNPPKKIKDILVDSSNYKYLNLNKYETTTFRETKSNIISRPLKNYTTFNSE |
| | NYVYNINGIGPTLTASGANSRIKIETQQGVRYLTPLECFKYMQFDVNDFKKVQSTNLISEN |
| | KMIYIAGRSIPVKILEAIENTLEFVNNEELE |
| SEQ ID NO: 3 | GGSQSQNGKCEGCNPDKDEAPYYTHLGAGPDVAAIRTLMEERYGEKGKAIRIEKVIYTGKE |
| TETv | GKSSQGCPIAKWVYRRSSEEEKLLCLVRVRPNHTCETAVMVIAIMLWDGIPKLLASELYSE |
| | LTDILGKCGICTNRRCSQNETRNCCCQGENPETCGASFSFGCSWSMYYNGCKFARSKKPRK |
| | FRLHGAEPKEEERLGSHLQNLATVIAPIYKKLAPDAYNNQVEFEHQAPDCCLGLKEGRPFS |
| | GVTACLDFSAHSHRDQQNMPNGSTVVVTLNREDNREVGAKPEDEQFHVLPMYIIAPEDEFG |
| | STEGQEKKIRMGSIEVLQSFRRRRVIRIGELPKSCEVSGQDAAAVQEIEYWSDSEHNFQDP |
| | CIGGVAIAPTHGSILIECAKCEVHATTKVNDPDRNHPTRISLVLYRHKNLFLPKHCLALWE |
| | AKMAEKARKEEECGKNGSDHVSQKNHGKQEKREPTGPQEPSYLRFIQSLAENTGSVTTDST |
| | VTTSPYAFTQVTGPYNTFV |
| SEQ ID NO: 4 | QSQNGKCEGCNPDKDEAPYYTHLGAGPDVAAIRTLMEERYGEKGKAIRIEKVIYTGKEGKS |
| TETcd | SQGCPIAKWVYRRSSEEEKLLCLVRVRPNHTCETAVMVIAIMLWDGIPKLLASELYSELTD |
| | ILGKCGICTNRRCSQNETRNCCCQGENPETCGASFSFGCSWSMYYNGCKFARSKKPRKFRL |
| | HGAEPKEEERLGSHLQNLATVIAPIYKKLAPDAYNNQVEFEHQAPDCCLGLKEGRPFSGVT |
| | ACLDFSAHSHRDQQNMPNGSTVVVTLNREDNREVGAKPEDEQFHVLPMYIIAPEDEFGSTE |
| | GQEKKIRMGSIEVLQSFRRRRVIRIGELPKSCKKKAEPKKAKTKKAARKRSSLENCSSRTE |
| | KGKSSSHTKLMENASHMKQMTAQPQLSGPVIRQPPTLQRHLQQGQRPQQPQPPQPQPQTTP |
| | QPQPQPQHIMPGNSQSVGSHCSGSTSVYTRQPTPHSPYPSSAHTSDIYGDTNHVNFYPTSS |
| | HASGSYLNPSNYMNPYLGLLNQNNQYAPFPYNGSVPVDNGSPFLGSYSPQAQSRDLHRYPN |
| | QDHLTNQNLPPIHTLHQQTFGDSPSKYLSYGNQNMQRDAFTTNSTLKPNVHHLATFSPYPT |
| | PKMDSHFMGAASRSPYSHPHTDYKTSEHHLPSHTIYSYTAAASGSSSSHAFHNKENDNIAN |
| | GLSRVLPGFNHDRTASAQELLYSLTGSSQEKQPEVSGQDAAAVQEIEYWSDSEHNFQDPCI |
| | GGVAIAPTHGSILIECAKCEVHATTKVNDPDRNHPTRISLVLYRHKNLFLPKHCLALWEAK |
| | MAEKARKEEECGKNGSDHVSQKNHGKQEKREPTGPQEPSYLRFIQSLAENTGSVTTDSTVT |
| | TSPYAFTQVTGPYNTFV |
| SEQ ID NO: 5 | MDYKDDDDKHMGGSDFPSCRCVEQIIEKDEGPFYTHLGAGPNVAAIREIMEERFGQKGKA |
| wild type | IRIERVIYTGKEGKSSQGCPIAKWVVRRSSSEEKLLCLVRERAGHTCEAAVIVILILVWE |
| TET2 | GIPLSLADKLYSELTETLRKYGTLTNRRCALNEERTCACQGLDPETCGASFSFGCSWSMY |
| catalytic | YNGCKFARSKIPRKFKLLGDDPKEEEKLESHLQNLSTLMAPTYKKLAPDAYNNQIEYEHR |
| domain | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLVFYQHKSMNEPKHGLALWEAKMAEKAREKEEECEKYG |
| SEQ ID NO: 6 | MDYKDDDDKHMGGSDFPSCRCVEQIIEKDEGPFYTHLGAGPNVAAIREIMEERFGQKGKA |
| TET2 V1900A | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASESFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLAFYQHKSMNEPKHGLALWEAKMAEKAREKEEECEKYG |
| SEQ ID NO: 7 | MDYKDDDDKHMGGSDFPSCRCVEQIIEKDEGPFYTHLGAGPNVAAIREIMEERFGQKGKA |
| TET2 V1900C | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASFSFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLCFYQHKSMNEPKHGLALWEAKMAEKAREKEEECEKYG |
| SEQ ID NO: 8 | MDYKDDDDKHMGGSDFPSCRCVEQIIEKDEGPFYTHLGAGPNVAAIREIMEERFGQKGKA |
| TET2 V1900G | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASESFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLGFYQHKSMNEPKHGLALWEAKMAEKAREKEEECEKYG |
| SEQ ID NO: 9 | MDYKDDDDKHMGGSDFPSCRCVEQIIEKDEGPFYTHLGAGPNVAAIREIMEERFGQKGKA |
| TET2 V1900I | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASESFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLIFYQHKSMNEPKHGLALWEAKMAEKAREKEEECEKYG |
| SEQ ID NO: | MDYKDDDDKHMGGSDFPSCRCVEQIIEKDEGPFYTHLGAGPNVAAIREIMEERFGQKGKA |
| 10 | IRIERVIYTGKEGKSSQGCPIAKWVVRRSSSEEKLLCLVRERAGHTCEAAVIVILILVWE |
| TET2 V1900P | GIPLSLADKLYSELTETLRKYGTLTNRRCALNEERTCACQGLDPETCGASFSFGCSWSMY |
| catalytic | YNGCKFARSKIPRKFKLLGDDPKEEEKLESHLQNLSTLMAPTYKKLAPDAYNNQIEYEHR |
| domain | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGGGGGSGGGGSDEVWSDSEQSFLDPDIGGVAVAPTHGSILIECAKRELHA |
| | TTPLKNPNRNHPTRISLPFYQHKSMNEPKHGLALWEAKMAEKAREKEEECEKYG |
| SEQ ID NO: | MDYKDDDDKHMGGSDFPSCRCVEQIIEKDEGPFYTHLGAGPNVAAIREIMEERFGQKGKAI |
| 11 | RIERVIYTGKEGKSSQGCPIAKWVVRRSSSEEKLLCLVRERAGHTCEAAVIVILILVWEGI |
| TET2-CS- | PLSLADKLYSELTETLRKYGTLTNRRCALNEERTCACQGLDPETCGASFSFGCSWSMYYNG |
| T1372S | CKFARSKIPRKFKLLGDDPKEEEKLESHLQNLSTLMAPTYKKLAPDAYNNQIEYEHRAPEC |
| | RLGLKEGRPFSGVSACLDFCAHAHRDLHNMQNGSTLVCTLTREDNREFGGKPEDEQLHVLP |
| | LYKVSDVDEFGSVEAQEEKKRSGAIQVLSSFRRKVRMLAEPVKTCRQRKLEAKKAAAEKLS |
| | GGGGSGGGGSGGGGSDEVWSDSEQSFLDPDIGGVAVAPTHGSILIECAKRELHATTPLKNP |
| | NRNHPTRISLVFYQHKSMNEPKHGLALWEAKMAEKAREKEEECEKYG |
| SEQ ID NO: | MGGSDFPSCRCVEQIIEKDEGPFYTHLGAGPNVAAIREIMEERFGQKGKAIRIERVIYTGK |
| 12 | EGKSSQGCPIAKWVVRRSSSEEKLLCLVRERAGHTCEAAVIVILILVWEGIPLSLADKLYS |
| TET2-CD- | ELTETLRKYGTLTNRRCALNEERTCACQGLDPETCGASFSFGCSWSMYYNGCKFARSKIPR |
| T1372S | KFKLLGDDPKEEEKLESHLQNLSTLMAPTYKKLAPDAYNNQIEYEHRAPECRLGLKEGRPF |
| | SGVSACLDFCAHAHRDLHNMQNGSTLVCTLTREDNREFGGKPEDEQLHVLPLYKVSDVDEF |
| | GSVEAQEEKKRSGAIQVLSSFRRKVRMLAEPVKTCRQRKLEAKKAAAEKLSSLENSSNKNE |
| | KEKSAPSRTKQTENASQAKQLAELLRLSGPVMQQSQQPQPLQKQPPQPQQQQRPQQQQPHH |
| | PQTESVNSYSASGSTNPYMRRPNPVSPYPNSSHTSDIYGSTSPMNFYSTSSQAAGSYLNSS |
| | NPMNPYPGLLNQNTQYPSYQCNGNLSVDNCSPYLGSYSPQSQPMDLYRYPSQDPLSKLSLP |
| | PIHTLYQPRFGNSQSFTSKYLGYGNQNMQGDGFSSCTIRPNVHHVGKLPPYPTHEMDGHFM |
| | GATSRLPPNLSNPNMDYKNGEHHSPSHIIHNYSAAPGMENSSLHALHLQNKENDMLSHTAN |
| | GLSKMLPALNHDRTACVQGGLHKLSDANGQEKQPLALVQGVASGAEDNDEVWSDSEQSFLD |
| | PDIGGVAVAPTHGSILIECAKRELHATTPLKNPNRNHPTRISLVFYQHKSMNEPKHGLALW |
| | EAKMAEKAREKEEECEKYGPDYVPQKSHGKKVKREPAEPHETSEPTYLRFIKSLAERTMSV |
| | TTDSTVTTSPYAFTRVTGPYNRYI |
| SEQ ID NO: | FSGVTACLD |
| 13 | |
| SEQ ID NO: | FSGVSACLD |
| 14 | |
| TET2 T1372S | |

### IV. Kits

Also provided are kits, e.g., comprising the oligonucleotide probes as described herein. The kits can be useful in, or for use in, performing the methods as described herein.

In some embodiments, the kit comprises one or more oligonucleotide probes that preferentially form a substrate for extension with a wild-type sequence relative to a variant sequence, that preferentially form a substrate for extension with a target nucleic acid that comprises a converted nucleotide relative to a target nucleic acid that does not comprise the converted nucleotide, or that preferentially form a substrate for extension with a target nucleic acid that does not comprise a converted nucleotide relative to a target nucleic acid that comprises the converted nucleotide.

In some embodiments, the kit comprises target-specific probes that specifically bind to epigenetic and/or sequence-variable target region sets, wherein the target-specific probes of at least one epigenetic target region set bind to target regions that are differentially methylated in different immune cell types. In some such embodiments, the target-specific probes comprise a capture moiety. In some embodiments, the kit comprises a solid support linked to a binding partner of the capture moiety.

In some embodiments, a kit comprises an agent that recognizes methyl cytosine in DNA. In some such embodiments, the agent is an antibody or a methyl binding protein or methyl binding domain.

In some embodiments, a kit comprises reagents for a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity. The procedure that affects a first nucleobase of the DNA differently from a second nucleobase of the DNA may be any of the procedures described elsewhere herein.

In some embodiments, the kit comprises one or more conversion reagents. The conversion reagents may comprise reagents for any combination of steps described herein, including but not limited to, in the numbered embodiments above and in any one of the workflows shown in the figures. In some embodiments, the kit comprises a TET2 enzyme comprising a T1372S mutation and a substituted borane reducing agent. The enzyme and reducing agent may be according to any of the embodiments thereof described elsewhere herein.

In some embodiments, the kit comprises adapters. In some embodiments, the kit comprises PCR primers, wherein the PCR primers anneal to a target region or to an adapter. In some embodiments, the kit comprises additional elements elsewhere herein. In some embodiments, the kit comprises instructions for performing a method described herein.

Kits may further comprise a plurality of oligonucleotide probes that selectively hybridize to least 5, 6, 7, 8, 9, 10, 20, 30, 40 or all genes selected from the group consisting of ALK, APC, BRAF, CDKN2A, EGFR, ERBB2, FBXW7, KRAS, MYC, NOTCH1, NRAS, PIK3CA, PTEN, RBI, TP53, MET, AR, ABL1, AKT1, ATM, CDH1, CSFIR, CTNNB1, ERBB4, EZH2, FGFR1, FGFR2, FGFR3, FLT3, GNA11, GNAQ, GNAS, HNF1A, HRAS, IDH1, IDH2, JAK2, JAK3, KDR, KIT, MLH1, MPL, NPM1, PDGFRA, PROC, PTPN11, RET, SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNE1, ESR1, RIT1, GATA3, MAP2K1, RHEB, ROS1, ARAF, MAP2K2, NFE2L2, RHOA, and NTRK1. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

The oligonucleotide probes can selectively hybridize to exon regions of the genes, e.g., of the at least 5 genes. In some cases, the oligonucleotide probes can selectively hybridize to at least 30 exons of the genes, e.g., of the at least 5 genes. In some cases, the multiple probes can selectively hybridize to each of the at least 30 exons. The probes that hybridize to each exon can have sequences that overlap with at least 1 other probe. In some embodiments, the oligoprobes can selectively hybridize to non-coding regions of genes disclosed herein, for example, intronic regions of the genes. The oligoprobes can also selectively hybridize to regions of genes comprising both exonic and intronic regions of the genes disclosed herein.

Any number of exons can be targeted by the oligonucleotide probes. For example, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 400, 500, 600, 700, 800, 900, 1,000, or more, exons can be targeted.

The kit can comprise at least 4, 5, 6, 7, or 8 different library adapters having distinct molecular barcodes and identical sample barcodes. The library adapters may not be sequencing adapters. For example, the library adapters do not include flow cell sequences or sequences that permit the formation of hairpin loops for sequencing. The different variations and combinations of molecular barcodes and sample barcodes are described throughout, and are applicable to the kit. Further, in some cases, the adapters are not sequencing adapters. Additionally, the adapters provided with the kit can also comprise sequencing adapters. A sequencing adapter can comprise a sequence hybridizing to one or more sequencing primers. A sequencing adapter can further comprise a sequence hybridizing to a solid support, e.g., a flow cell sequence. For example, a sequencing adapter can be a flow cell adapter. The sequencing adapters can be attached to one or both ends of a polynucleotide fragment. In some cases, the kit can comprise at least 8 different library adapters having distinct molecular barcodes and identical sample barcodes. The library adapters may not be sequencing adapters. The kit can further include a sequencing adapter having a first sequence that selectively hybridizes to the library adapters and a second sequence that selectively hybridizes to a flow cell sequence. In another example, a sequencing adapter can be hairpin shaped. For example, the hairpin shaped adapter can comprise a complementary double stranded portion and a loop portion, where the double stranded portion can be attached (e.g., ligated) to a double-stranded polynucleotide. Hairpin shaped sequencing adapters can be attached to both ends of a polynucleotide fragment to generate a circular molecule, which can be sequenced multiple times. A sequencing adapter can be up to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases from end to end. The sequencing adapter can comprise 20-30, 20-40, 30-50, 30-60, 40-60, 40-70, 50-60, 50-70, bases from end to end. In a particular example, the sequencing adapter can comprise 20-30 bases from end to end. In another example, the sequencing adapter can comprise 50-60 bases from end to end. A sequencing adapter can comprise one or more barcodes. For example, a sequencing adapter can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., at least 8 bases. The barcode can be contiguous or non-contiguous sequences, as described above.

The library adapters can be blunt ended and Y-shaped and can be less than or equal to 40 nucleic acid bases in length. The adapters can comprise one or more restriction endonuclease cleavage sites as described herein. Other variations of the can be found throughout and are applicable to the kit.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

### EXAMPLES

### Example 1: Selective Depletion of Target DNAs Comprising a Wild-Type Sequence

The workflow described in this example is illustrated in FIG. 1. A set of patient samples is analyzed by an NGS assay to detect the presence/absence of cancer. DNA (a population of target DNA molecules) is extracted from these samples. Y-shaped adapters (such as Illumina Y-shaped adapters) comprising a 5′-GCTCTTCN-3′ (SEQ ID NO: 11) restriction enzyme cleavage site (a BpsQI recognition site) and a universal primer binding site are ligated to the population of target DNA molecules at both ends of the molecules. The DNA molecules are then contacted with first and second oligonucleotide probes comprising sequences complementary to a wild-type sequence at a locus of interest on both strands of a target nucleic acid. The locus of interest comprises a portion of the KRAS gene 3′ of and including a single nucleotide variant (SNV) that is causative of a guanosine substitution corresponding to position 12 (G12X) in the KRAS amino acid sequence. The G12X substitution has been associated with various cancers. See, e.g., Parikh et al., Journal of Hematology & Oncology, 2022, 15:152; Pantsar et al., PLOS Computational Biology, 2018, 14(9):e1006458. The 3′-most nucleotide of the oligonucleotide probe aligns with and is complementary to the wild-type nucleotide at the locus. The 3′ end of the oligonucleotide probe can therefore anneal to the wild-type sequence at the locus, and can be extended using a DNA polymerase that lacks 3′ to 5′ exonuclease activity. On the other hand, the 3′ end of the oligonucleotide probe does not anneal to a target DNA molecule bearing a SNV at the same locus, and thus cannot be extended. The oligonucleotide probe therefore preferentially forms a substrate for extension with target DNA molecules comprising the wild-type sequence (e.g., a G or a C nucleotide) relative to target DNA molecules comprising the SNV (e.g., a T or an A nucleotide) at the same locus.

Extension of the oligonucleotide probe produces a partially double-stranded DNA molecule comprising the wild-type sequence. As a result of the extension, the 5′ adapter in the DNA molecule is double stranded. Because the oligonucleotide probe preferentially formed a substrate for extension with the target DNA molecules comprising the wild-type sequence, substantially no target DNA molecules are extended that comprise the SNV. The partially double-stranded DNAs comprising the wild-type sequence are selectively digested using the BspQI restriction endonuclease that recognizes the 5′-GCTCTTCN-3′ (SEQ ID NO: 11) cleavage site present in the double stranded adapter. The target DNA molecules that were not cleaved by the restriction endonuclease (i.e., the target DNA molecules comprising the SNV) are then amplified exponentially, while the target DNA molecules that were cleaved (i.e., the target DNA molecules comprising the wild-type sequence) are amplified linearly, thereby selectively depleting the target DNA molecules comprising the wild-type sequence and increasing the relative proportion of target DNA molecules comprising the SNV (i.e., enriching for DNAs comprising the SNV) as compared to target DNA molecules comprising the wild-type sequence. The target DNA molecules are then sequenced to determine the presence/absence of cancer in the subject, based wholly or in part on detection and/or quantification of the SNV.

### Example 2: Selective Depletion of Target RNAs Comprising a Wild-Type Sequence

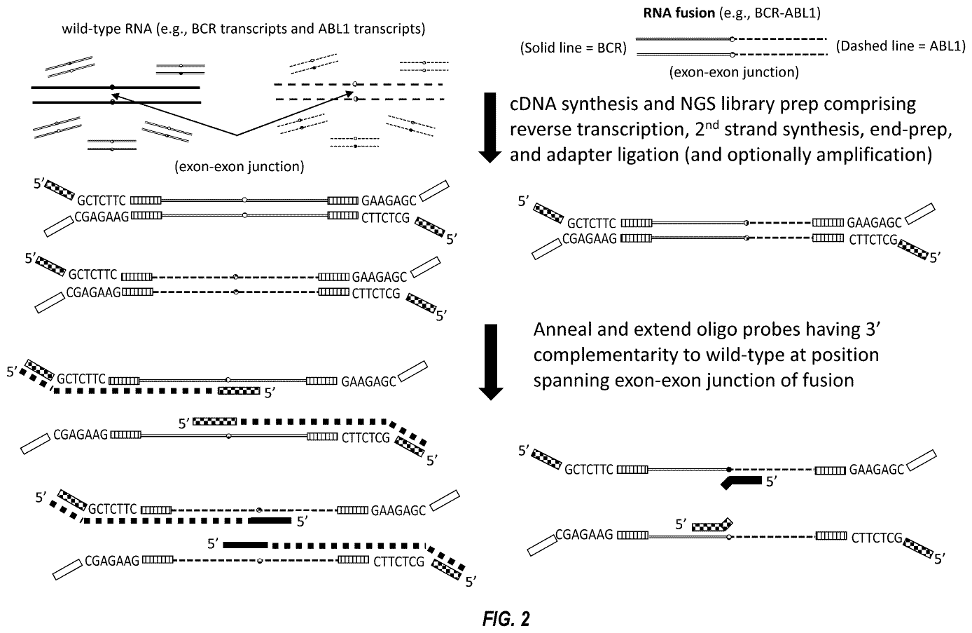

The workflow described in this example is illustrated in FIG. 2. A set of patient samples is analyzed by an NGS assay to detect the presence/absence of cancer. RNA is extracted from these samples. cDNA is produced from the RNA, thereby providing a population of target cDNA molecules that comprise cDNAs comprising a wild-type sequence and cDNAs comprising one or more variant nucleotides at a particular locus. Y-shaped adapters (such as Illumina Y-shaped adapters) comprising a 5′-GCTCTTCN-3′ restriction enzyme cleavage site (a BpsQI recognition site) and a universal primer binding site are ligated to the population of target cDNA molecules at both ends of the molecules. The cDNA molecules are then contacted with two sets of first and second oligonucleotide probes comprising sequences complementary to a wild-type sequence at a locus of interest on both strands of a target nucleic acid. The locus of interest comprises an exon-exon junction of the BCR-ABL1 gene fusion The BCR-ABL1 fusion (also known as the Philadelphia chromosome) has been associated with leukemias. See, e.g., Roskoski, Pharmacological Research, 2022, 178:106156. The 3′-most nucleotide of an oligonucleotide probe aligns with and is complementary to a wild-type nucleotide at the location of the BCR-ABL1 fusion junction, i.e., to the native (non-fused) BCR or ABL1 sequences. The 3′ end of the oligonucleotide probe can therefore anneal to a wild-type sequence at the locus, and can be extended using a DNA polymerase that lacks 3′ to 5′ exonuclease activity. On the other hand, the 3′ end of the oligonucleotide probe does not anneal to a target cDNA molecule bearing the fusion junction at the same locus, and thus cannot be extended. The oligonucleotide probe therefore preferentially forms a substrate for extension with target cDNA molecules comprising the wild-type sequence (e.g., a G or a C nucleotide) relative to target cDNA molecules comprising the fusion (e.g., a T or an A nucleotide) at the same locus.

Extension of the oligonucleotide probe produces a partially double-stranded cDNA molecule comprising the wild-type sequence. As a result of the extension, the 5′ adapter in the cDNA molecule is double stranded. Because the oligonucleotide probe preferentially formed a substrate for extension with the target cDNA molecules comprising the wild-type sequence, substantially no target cDNA molecules are extended that comprise the fusion. The partially double-stranded cDNAs comprising the wild-type sequence are selectively digested using the BspQI restriction endonuclease that recognizes the 5′-GCTCTTCN-3′ cleavage site present in the double stranded adapter. The target cDNA molecules that were not cleaved by the restriction endonuclease (i.e., the target cDNA molecules comprising the fusion) are then amplified exponentially, while the target cDNA molecules that were cleaved (i.e., the target cDNA molecules comprising the wild-type sequence) are amplified linearly, thereby selectively depleting the target cDNA molecules comprising the wild-type sequence and increasing the relative proportion of target cDNA molecules comprising the fusion (i.e., enriching for target cDNA molecules comprising the fusion) as compared to target cDNA molecules comprising the wild-type sequence. The target cDNA molecules are then sequenced to determine the presence/absence of cancer in the subject, based wholly or in part on detection and/or quantification of the fusion.

### Example 3: Selective Depletion of Target DNAs that do not Comprise a Converted Nucleotide

In this example, a set of patient samples is analyzed by an NGS assay to detect the presence/absence of cancer. DNA (a population of target DNA molecules) is extracted from these samples and then subjected to a procedure that modifies a first nucleobase of the DNA differently than a second nucleobase. For example, the DNA is treated with bisulfite in order to convert unmodified cytosines to uracils.

After base conversion, Y-shaped adapters (such as Illumina Y-shaped adapters) comprising a 5′-GCTCTTCN-3′ restriction enzyme cleavage site (a BpsQI recognition site) and a universal primer binding site are ligated to the population of target DNA molecules at both ends of the molecules. The DNA molecules are then contacted with first and second oligonucleotide probes comprising sequences complementary to both strands of a sequence that does not comprise a converted base at a locus of interest. A methylated cytosine at the locus of interest has been associated with one or more cancers. The 3′-most nucleotide of the oligonucleotide probe aligns with and is complementary to the unconverted base (e.g., a cytosine that was not converted to a uracil) at the locus. The 3′ end of the oligonucleotide probe can therefore anneal to the unconverted base at the locus, and can be extended using a DNA polymerase that lacks 3′ to 5′ exonuclease activity. On the other hand, the 3′ end of the oligonucleotide probe does not anneal to a target DNA molecule bearing a converted base (e.g., a uracil) at the same locus, and thus cannot be extended. The oligonucleotide probe therefore preferentially forms a substrate for extension with target DNA molecules that do not comprise the converted base relative to target DNA molecules comprising the unconverted base (e.g., a U nucleotide at the same locus.

Extension of the oligonucleotide probe produces a partially double-stranded DNA molecule that does not comprise the converted base. As a result of the extension, the 5′ adapter in the DNA molecule is double stranded. Because the oligonucleotide probe preferentially formed a substrate for extension with the target DNA molecules that do not comprise the converted base, substantially no target DNA molecules are extended that comprise the converted base. The partially double-stranded DNAs that do not comprise the converted base are selectively digested using the BspQI restriction endonuclease that recognizes the 5′-GCTCTTCN-3′ cleavage site present in the double stranded adapter. The target DNA molecules that were not cleaved by the restriction endonuclease (i.e., the target DNA molecules comprising the converted base) are then amplified exponentially, while the target DNA molecules that were cleaved (i.e., the target DNA molecules that do not comprise the converted base) are amplified linearly, thereby selectively depleting the target DNA molecules comprising the converted base and increasing the relative proportion of target DNA molecules that do not comprise the converted base (i.e., enriching for DNA molecules that do not comprise the converted base) as compared to target DNA molecules comprising the converted base. The target DNA molecules are then sequenced to determine the presence/absence of cancer in the subject, based wholly or in part on detection and/or quantification of the unconverted base.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

What is claimed is:

1. A method of selectively depleting a target nucleic acid comprising a wild-type sequence, the method comprising:
(a) contacting a population of target nucleic acids with an oligonucleotide probe, wherein:
(i) the population of target nucleic acids comprises a target nucleic acid comprising a wild-type sequence and comprises or is suspected of comprising a target nucleic acid comprising a variant sequence; and
(ii) the oligonucleotide probe preferentially forms a substrate for extension with the wild-type sequence relative to the variant sequence;
(b) extending the oligonucleotide probe, wherein the extending produces a partially double-stranded target nucleic acid comprising the wild-type sequence; and
(c) selectively digesting the partially double-stranded target nucleic acid comprising the wild-type sequence;
thereby selectively depleting the target nucleic acid comprising the wild-type sequence.

2. A method of selectively depleting a target nucleic acid that comprises a converted nucleotide, the method comprising:
(a) subjecting a population of target nucleic acids from a subject to a conversion procedure that affects a first nucleobase of the target nucleic acids differently from a second nucleobase of the target nucleic acids, wherein the first nucleobase is a modified or unmodified nucleobase, the second nucleobase is a modified or unmodified nucleobase different from the first nucleobase, and the first nucleobase and the second nucleobase have the same base pairing specificity, thereby providing a population of converted target nucleic acids;
(b) contacting the population of converted target nucleic acids with an oligonucleotide probe, wherein the oligonucleotide probe preferentially forms a substrate for extension with a target nucleic acid that comprises a converted nucleotide relative to a target nucleic acid that does not comprise the converted nucleotide;
(c) extending the oligonucleotide probe, wherein the extending produces a partially double-stranded target nucleic acid that comprises the converted nucleotide; and
(d) selectively digesting the partially double-stranded target nucleic acid that comprises the converted nucleotide;
thereby selectively depleting the target nucleic acid that comprises the converted nucleotide.

3. A method of selectively depleting a target nucleic acid that does not comprise a converted nucleotide, the method comprising:
(a) subjecting a population of target nucleic acids to a conversion procedure that affects a first nucleobase of the target nucleic acids differently from a second nucleobase of the target nucleic acids, wherein the first nucleobase is a modified or unmodified nucleobase, the second nucleobase is a modified or unmodified nucleobase different from the first nucleobase, and the first nucleobase and the second nucleobase have the same base pairing specificity, thereby providing a population of converted target nucleic acids;
(b) contacting the population of converted target nucleic acids with an oligonucleotide probe, wherein the oligonucleotide probe preferentially forms a substrate for extension with a target nucleic acid that does not comprise a converted nucleotide relative to a target nucleic acid that comprises the converted nucleotide;
(c) extending the oligonucleotide probe, wherein the extending produces a partially double-stranded target nucleic acid that does not comprise the converted nucleotide; and
(d) selectively digesting the partially double-stranded target nucleic acid that does not comprise the converted nucleotide;
thereby selectively depleting the target nucleic acid that does not comprise the converted nucleotide.

4. The method of any one of the preceding claims, further comprising sequencing the population of target nucleic acids after selectively digesting the partially double-stranded target nucleic acid comprising the wild-type sequence, the partially double-stranded target nucleic acid that comprises the converted nucleotide, or the partially double-stranded target nucleic acid that does not comprise the converted nucleotide.

5. A method of selectively depleting a target nucleic acid comprising a wild-type sequence, the method comprising:
(a) contacting a population of target nucleic acids with an oligonucleotide probe, wherein:
(i) substantially each target nucleic acid of the population of target nucleic acids is single-stranded and comprises an adapter comprising a restriction enzyme cleavage site and a universal primer binding site;
(ii) the population of target nucleic acids comprises a target nucleic acid comprising a wild-type sequence and comprises or is suspected of comprising a target nucleic acid comprising a variant sequence; and
(iii) the oligonucleotide probe preferentially forms a substrate for extension with the wild-type sequence relative to the variant sequence;
(b) extending the oligonucleotide probe, wherein the extending produces a partially double-stranded target nucleic acid comprising the wild-type sequence;
(c) selectively digesting the partially double-stranded target nucleic acid comprising the wild-type sequence, thereby producing a selectively digested population of target nucleic acids;
(d) amplifying the selectively digested population of target nucleic acids exponentially, thereby producing a population of amplified target nucleic acids; and
(e) sequencing the population of amplified target nucleic acids.

6. A method of selectively depleting a target nucleic acid that comprises a converted nucleotide, the method comprising:
(a) subjecting a population of target nucleic acids to a conversion procedure that affects a first nucleobase of the target nucleic acids differently from a second nucleobase of the target nucleic acids, wherein
(i) substantially each target nucleic acid of the population of target nucleic acids is single-stranded and comprises an adapter comprising a restriction enzyme cleavage site and a universal primer binding site; and
(ii) the first nucleobase is a modified or unmodified nucleobase, the second nucleobase is a modified or unmodified nucleobase different from the first nucleobase, and the first nucleobase and the second nucleobase have the same base pairing specificity;
thereby providing a population of converted target nucleic acids;
(b) contacting the population of converted target nucleic acids with an oligonucleotide probe, wherein the oligonucleotide probe preferentially forms a substrate for extension with a target nucleic acid that comprises a converted nucleotide relative to a target nucleic acid that does not comprise the converted nucleotide;
(c) extending the oligonucleotide probe, wherein the extending produces a partially double-stranded target nucleic acid that comprises the converted nucleotide; and
(d) selectively digesting the partially double-stranded target nucleic acid that comprises the converted nucleotide, thereby producing a selectively digested population of target nucleic acids;
(e) amplifying the selectively digested population of target nucleic acids exponentially, thereby producing a population of amplified target nucleic acids; and
(f) sequencing the population of amplified target nucleic acids.

7. A method of selectively depleting a target nucleic acid that does not comprise a converted nucleotide, the method comprising:
(a) subjecting a population of target nucleic acids to a conversion procedure that affects a first nucleobase of the target nucleic acids differently from a second nucleobase of the target nucleic acids, wherein
(i) substantially each target nucleic acid of the population of target nucleic acids is single-stranded and comprises an adapter comprising a restriction enzyme cleavage site and a universal primer binding site; and
(ii) the first nucleobase is a modified or unmodified nucleobase, the second nucleobase is a modified or unmodified nucleobase different from the first nucleobase, and the first nucleobase and the second nucleobase have the same base pairing specificity;
thereby providing a population of converted target nucleic acids;
(b) contacting the population of converted target nucleic acids with an oligonucleotide probe, wherein the oligonucleotide probe preferentially forms a substrate for extension with a target nucleic acid that does not comprise a converted nucleotide relative to a target nucleic acid that comprises the converted nucleotide;
(c) extending the oligonucleotide probe, wherein the extending produces a partially double-stranded target nucleic acid that does not comprise the converted nucleotide; and
(d) selectively digesting the partially double-stranded target nucleic acid that does not comprise the converted nucleotide, thereby producing a selectively digested population of target nucleic acids;
(e) amplifying the selectively digested population of target nucleic acids exponentially, thereby producing a population of amplified target nucleic acids; and
(f) sequencing the population of amplified target nucleic acids.

8. The method of any one of the claim 1, 4, or 5, wherein the population of target nucleic acids comprises the target nucleic acid comprising the variant sequence.

9. The method of any one of the preceding claims, wherein
(a) the extending does not produce a partially double-stranded target nucleic acid comprising the variant sequence;
(b) the method selectively depletes a target nucleic acid that comprises the converted nucleotide and the extending does not produce a partially double-stranded target nucleic acid that does not comprise the converted nucleotide; or
(c) the method selectively depletes the target nucleic acid that does not comprise the converted nucleotide and the extending does not produce a partially double-stranded target nucleic acid that comprises the converted nucleotide.

10. The method of any one of the preceding claims, wherein a first portion of the oligonucleotide probe
(a) is complementary to a portion of the target nucleic acid comprising the wild-type sequence, and is not complementary to a portion of the target nucleic acid comprising the variant sequence;
(b) is complementary to a portion of the target nucleic acid that does not comprise the converted nucleotide, and is not complementary to a portion of the target nucleic acid that comprises the converted nucleotide; or
(c) is complementary to a portion of the target nucleic acid that comprises the converted nucleotide, and is not complementary to a portion of the target nucleic acid that does not comprise the converted nucleotide.

11. The method of any one of the preceding claims, wherein a second portion of the oligonucleotide probe
(a) is complementary to both a portion of the target nucleic acid comprising the wild-type sequence and a portion of the target nucleic acid comprising the variant sequence; or
(b) is complementary to both a portion of the target nucleic acid that does not comprise the converted nucleotide and a portion of the target nucleic acid that comprises the converted nucleotide.

12. The method of claim 10 or claim 11, wherein the first portion of the oligonucleotide probe comprises a 3′ end of the oligonucleotide probe.

13. The method of any one of claims 10-12, wherein the second portion of the oligonucleotide probe comprises a 5′ end of the oligonucleotide probe.

14. The method of any one of claims 2, 3, 6, 7, or 6-13, wherein the method selectively depletes a target nucleic acid that comprises the converted nucleotide and the converted nucleotide is bound by the 3′ nucleotide of the oligonucleotide probe, or the method selectively depletes the target nucleic acid that does not comprise the converted nucleotide and the converted nucleotide is not bound by the 3′ nucleotide of the oligonucleotide probe.

15. The method of any one of the preceding claims, further comprising attaching one or more adapters to the target nucleic acid, optionally wherein the attaching comprises ligating.

16. The method of any one of the preceding claims, wherein the target nucleic acid comprises a 5′ adapter, a 3′ adapter, or both a 5′ adapter and a 3′ adapter.

17. The method of the immediately preceding claim, wherein the 5′ adapter, or both the 5′ adapter and the 3′ adapter comprise at least one sequence that is recognized by at least one restriction enzyme.

18. The method of claim 16 or claim 17, wherein the 5′ adapter is downstream of an oligonucleotide probe binding site within the target nucleic acid.

19. The method of any one of claims 16-18, wherein the 5′ adapter, or both the 5′ adapter and the 3′ adapter, comprises a universal primer binding site.

20. The method of any one of claims 16-19, wherein the 5′ adapter, or both the 5′ adapter and the 3′ adapter, comprises a barcode.

21. The method of any one of the preceding claims, further comprising contacting the population of nucleic acids with a second oligonucleotide probe complementary to the strand of the target nucleic acid opposite the strand to which the first oligonucleotide probe is complementary.

22. The method of any one of the preceding claims, wherein extending the oligonucleotide probe comprises contacting the substrate for extension with a DNA polymerase that lacks 3′->5′ exonuclease activity.

23. The method of any one of the preceding claims, wherein selectively digesting the partially double-stranded target nucleic acid comprising the wild-type sequence, the partially double-stranded target nucleic acid that comprises the converted nucleotide, or the partially double-stranded target nucleic acid that does not comprise the converted nucleotide results in substantially linear amplification of the selectively digested target nucleic acid.

24. The method of any one of the preceding claims, wherein a concentration of the target nucleic acid comprising the variant sequence is increased relative to a concentration of the target nucleic acid comprising the wild-type sequence, a concentration of the target nucleic acid comprising the converted nucleotide is increased relative to a concentration of the target nucleic acid that does not comprise the converted nucleotide, or a concentration of the target nucleic acid that does not comprise the converted nucleotide is increased relative to a concentration of the target nucleic acid comprising the converted nucleotide.

25. The method of the immediately preceding claim, wherein
(a) a post-selective digestion ratio of the target nucleic acid comprising the variant sequence to the target nucleic acid comprising the wild-type sequence is at least 1500:1, 1000:1, 500:1, 100:1, 10:1, 5:1, 1:1, 2:1, 3:1, 4:1, 5:1, 10:1, 100:1, or 1000:1;
(b) a post-selective digestion ratio of the target nucleic acid comprising the unconverted nucleotide to the target nucleic acid comprising converted nucleotide is at least 1500:1, 1000:1, 500:1, 100:1, 10:1, 5:1, 1:1, 2:1, 3:1, 4:1, 5:1, 10:1, 100:1, or 1000:1; or
(c) a post-selective digestion ratio of the target nucleic acid comprising the converted nucleotide to the target nucleic acid comprising the unconverted nucleotide is at least 1500:1, 1000:1, 500:1, 100:1, 10:1, 5:1, 1:1, 2:1, 3:1, 4:1, 5:1, 10:1, 100:1, or 1000:1.

26. The method of any one of the preceding claims, wherein selectively digesting the partially double-stranded target nucleic acid comprising the wild-type sequence, selectively digesting the partially double-stranded target nucleic acid that comprises the converted nucleotide, or selectively digesting the partially double-stranded target nucleic acid that does not comprise the converted nucleotide comprises contacting the population of target nucleic acids with at least one restriction endonuclease.

27. The method of the immediately preceding claim, wherein the at least one restriction endonuclease cleaves a double-stranded nucleic acid and/or a partially double stranded nucleic acid, and does not cleave a single-stranded nucleic acid.

28. The method of any one of claims 17-27, wherein the at least one restriction endonuclease cleaves the partially double-stranded target nucleic acid at the at least one sequence within the at least one adapter.

29. The method of any one of claims 17-28, wherein the at least one restriction endonuclease is BspQI, AflIII, BsiHKAI, BtrI, MaeII, SduI, HpaII, BstUI, Hin6I, SsiI, or HpyCH4IV.

30. The method of any one of claims 17-29, wherein the at least one restriction endonuclease cleaves a double-stranded nucleic acid at a 5′-GCTCTTCN-3′ cleavage site in an adapter.

31. The method of any one of claims 17-30, wherein the at least one restriction endonuclease is BspQI.

32. The method of any one of claims 17-31, wherein the oligonucleotide probe, or the first and the second oligonucleotide probes, do not comprise the cleavage site.

33. The method of any one of the preceding claims, wherein the population of target nucleic acids comprises DNA or cDNA.

34. The method of any one of claims 1, 4, 5, 8-13, or 15-33, wherein the population of target nucleic acids comprises RNA and the method further comprises a cDNA synthesis step.

35. The method of any one of claims 1, 4, 5, 8-13, or 15-34, wherein the variant sequence is a single nucleotide variant (SNV), an insertion or deletion (indel), a translocation, a gene fusion, or an epigenetic modification.

36. The method of any one of claims 1, 4, 5, 8-13, or 15-35, wherein the variant sequence is an SNV, and wherein the oligonucleotide probe comprises a 3′ portion that is complementary to the target nucleic acid comprising the wild-type sequence and is not complementary to the target nucleic acid comprising the SNV.

37. The method of any one of claims 1, 4, 5, 8-13, or 15-35, wherein the variant sequence is an RNA fusion, and wherein the oligonucleotide probe comprises a 3′ portion that is complementary to the target nucleic acid comprising the wild-type sequence and is not complementary to the target nucleic acid comprising the gene fusion.

38. The method of any one of claims 1, 4, 5, 8-13, or 15-35, wherein the variant sequence is a translocation, and wherein the oligonucleotide probe comprises a 3′ portion that is complementary to the target nucleic acid comprising the wild-type sequence and is not complementary to the target nucleic acid comprising the translocation.

39. The method of any one of claims 2-4, 6, 7, or 14-33, wherein the variant sequence is an epigenetic modification, and wherein the oligonucleotide probe comprises a 3′ portion that is complementary to the target nucleic acid comprising the converted nucleotide and is not complementary to the target nucleic acid that does not comprise the converted nucleotide.

40. The method of any one of claims 2-4, 6, 7, or 14-33, wherein the variant sequence is an epigenetic modification, and wherein the oligonucleotide probe comprises a 3′ portion that is complementary to the target nucleic acid that does not comprise the converted nucleotide and is not complementary to the target nucleic acid comprising the converted nucleotide.

41. The method of any one of claims 36-40, wherein the 3′ portion of the oligonucleotide probe comprises one, two, three, four, or more than four of the 3′-most nucleotides of the oligonucleotide probe.

42. The method of any one of the preceding claims, further comprising partitioning the population of target nucleic acids into a plurality of subsamples, the plurality comprising a first subsample and a second subsample, wherein the partitioning is performed prior to the sequencing and/or
(a) prior to the selectively depleting the target nucleic acid comprising the wild-type sequence, the target nucleic acid comprising the converted nucleotide, or the target nucleic acid that does not comprise the converted nucleotide;
(b) prior to the amplifying the selectively digested population of target nucleic acids;
(c) prior to the subjecting the population of target nucleic acids to a procedure that affects a first nucleobase of the DNA differently from a second nucleobase of the DNA; and/or
(d) prior to a step of enriching for one or more sets of target regions of DNA.

43. The method of the immediately preceding claim, wherein the partitioning comprises partitioning on the basis of methylation level.

44. The method of claim 42 or claim 43, wherein the partitioning comprises contacting the population of target nucleic acids with an agent that recognizes a modified nucleobase in the DNA, wherein the first subsample comprises DNA with the modified nucleobase in a greater proportion than the second subsample.

45. The method of claim 44, wherein the agent that recognizes a modified nucleobase in the DNA is a methyl binding reagent.

46. The method of claim 45, wherein the methyl binding reagent is a methyl binding domain (MBD) protein or an antibody.

47. The method of claim 44 or claim 45, wherein the methyl binding reagent is specific to one or more methylated nucleotide bases, optionally wherein the one or more methylated nucleotide bases is 5-methylcytosine.

48. The method of any one of claims 45-47, wherein the methyl binding reagent is immobilized on a solid support.

49. The method of any one of claims 42-48, wherein the partitioning comprises immunoprecipitation of methylated DNA.

50. The method of any one of claims 42-49, wherein the partitioning comprises partitioning on the basis of binding to a protein, optionally wherein the protein is a methylated protein, an acetylated protein, an unmethylated protein, an unacetylated protein; and/or optionally wherein the protein is a histone.

51. The method of claim 50, wherein the partitioning comprises contacting the DNA with a binding reagent which is specific for the protein and is immobilized on a solid support.

52. The method of any one of claims 42-51, wherein a first partitioned subsample of the plurality of partitioned subsamples is differentially tagged from a second partitioned subsample of the plurality of partitioned subsamples.

53. The method of any one of claims 2-4, 6, 7, 14-33, or 39-52, wherein the first nucleobase is an unmodified cytosine and the second nucleobase is a modified cytosine, optionally wherein the modified cytosine is 5-methylcytosine or 5-hydroxymethylcytosine.

54. The method of any one of claims 1, 4, 5, 8-13, 15-38, or 41-53, wherein the method further comprises subjecting the DNA or a subsample thereof to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity, thereby providing a population of converted target nucleic acids.

55. The method of any one of claims 2-4, 6, 7, 14-33, or 39-54, wherein the procedure that affects a first nucleobase of the DNA differently from a second nucleobase of the DNA is performed prior to the sequencing and/or
(a) prior to the selectively depleting the target nucleic acid comprising the wild-type sequence, the target nucleic acid comprising the converted nucleotide, or the target nucleic acid that does not comprise the converted nucleotide;
(b) prior to the amplifying the selectively digested population of target nucleic acids;
(c) prior to the partitioning the population of target nucleic acids into a plurality of subsamples; and/or
(d) prior to a step of enriching for one or more sets of target regions of DNA.

56. The method of any one of claims 2-4, 6, 7, 14-33, or 39-55, wherein the first nucleobase is an unmodified cytosine and the second nucleobase is a modified cytosine, optionally wherein the modified cytosine is 5-methylcytosine or 5-hydroxymethylcytosine.

57. The method of any one of claims 2-4, 6, 7, 14-33, or 39-56, wherein the procedure that affects a first nucleobase of the DNA differently from a second nucleobase of the DNA chemically converts the first or second nucleobase such that the base pairing specificity of the converted nucleobase is altered.

58. The method of any one of claims 2-4, 6, 7, 14-33, or 39-57, wherein the procedure that affects a first nucleobase of the DNA differently from a second nucleobase of the DNA is methylation-sensitive conversion.

59. The method of claim 58, wherein the methylation-sensitive conversion is bisulfite conversion, oxidative bisulfite (Ox-BS) conversion, Tet-assisted bisulfite (TAB) conversion, APOBEC-coupled epigenetic (ACE) conversion, or enzymatic conversion.

60. The method of claim 59, wherein the Tet-assisted conversion further comprises a substituted borane reducing agent, optionally wherein the substituted borane reducing agent is 2-picoline borane, borane pyridine, tert-butylamine borane, or ammonia borane.

61. The method of any one of the preceding claims, further comprising enriching for one or more sets of target regions of DNA, RNA, or cDNA prepared from the RNA, wherein the one or more sets of target regions comprises one or more of a sequence-variable target region set and an epigenetic target region set, thereby providing enriched DNA, RNA, or cDNA.

62. The method of the immediately preceding claim, wherein the enriching is performed prior to the sequencing and/or
(a) prior to the selectively depleting the target nucleic acid comprising the wild-type sequence, the target nucleic acid comprising the converted nucleotide, or the target nucleic acid that does not comprise the converted nucleotide;
(b) prior to the amplifying the selectively digested population of target nucleic acids;
(c) prior to the partitioning the population of target nucleic acids into a plurality of subsamples; and/or
(d) prior to the subjecting the population of target nucleic acids to the conversion procedure that affects a first nucleobase of the DNA differently from a second nucleobase of the DNA.

63. The method of claim 61 or claim 62, wherein the enriching comprises contacting the DNA, RNA, or cDNA prepared from the RNA, with target-specific probes specific for the one or more sets of epigenetic target regions and/or for the one or more sets of sequence-variable target regions.

64. The method of any one of claims 61-63 wherein the epigenetic target region set comprises a hypermethylation variable target region set and/or a hypomethylation variable target region set.

65. The method of any one of claims 61-64, wherein the epigenetic target region set comprises a fragmentation variable target region set.

66. The method of claim 65, wherein the fragmentation variable target region set comprises transcription start site regions.

67. The method of claim 65 or 66, wherein the fragmentation variable target region set comprises CTCF binding regions.

68. The method of any one of claims 61-67, wherein the epigenetic target region set comprises one or more type-specific epigenetic target regions.

69. The method of claim 68, wherein the one or more type-specific epigenetic target regions comprises type-specific differentially methylated regions and/or type specific fragments.

70. The method of claim 68, wherein the one or more type-specific epigenetic target regions comprises type-specific hypomethylated regions and/or type-specific hypermethylated regions.

71. The method of any one of claims 68-70, wherein the one or more type-specific epigenetic target regions comprises cell-type specific, cell cluster-type specific, tissue-type specific, and/or cancer-type specific epigenetic target regions.

72. The method of any one of claims 68-71, wherein the one or more type-specific epigenetic target regions comprise target regions that are:
hypermethylated in immune cells relative to non-immune cell types present in a blood sample;
differentially methylated in colon relative to other tissue types;
differentially methylated in breast relative to other tissue types;
differentially methylated in liver relative to other tissue types;
differentially methylated in kidney relative to other tissue types;
differentially methylated in pancreas relative to other tissue types;
differentially methylated in prostate relative to other tissue types;
differentially methylated in skin relative to other tissue types; or
differentially methylated in bladder relative to other tissue types.

73. The method of any one of claims 70-72, wherein the hypermethylated target regions are methylated to an extent that is at least 10%, 20%, 30%, or at least 40% greater than the average methylation of the target regions in the sample or relative to other cell or tissue types.

74. The method of any one of claims 68-73, wherein the one or more type-specific epigenetic target regions comprises
target regions that are hypomethylated in non-immune cell types present in the sample relative to the methylation level of the target regions in a different cell or tissue type in the sample;
fragments specific to immune cells relative to non-immune cell types present in the sample; or
fragments specific to colon, lung, breast, liver, kidney, pancreas, prostate, skin, or bladder relative to other tissue types.

75. The method of any one of claims 68-74, wherein the level of the one or more type-specific epigenetic target regions that originated from a cell type or a tissue type is determined.

76. The method any one of claims 68-75, wherein the levels of the one or more type-specific epigenetic target regions that originated from one or more immune cells, non-immune cell types present in a blood sample, and/or colon, lung, breast, liver, kidney, prostate, skin, bladder, or pancreas cells are determined.

77. The method of any one of claims 68-76, further comprising identifying at least one cell type, cell cluster type, tissue type, and/or cancer type from which the one or more type-specific epigenetic target regions originated.

78. The method of any one of claims 68-77, comprising determining the methylation levels of the type-specific epigenetic target regions.

79. The method of any one of the preceding claims, wherein the DNA molecules are amplified.

80. The method of any one of the preceding claims, wherein the DNA molecules are amplified prior to the sequencing and/or
(a) prior to the selectively depleting the target nucleic acid comprising the wild-type sequence, the target nucleic acid comprising the converted nucleotide, or the target nucleic acid that does not comprise the converted nucleotide;
(b) prior to the enriching for one or more sets of target regions of DNA;
(c) prior to the partitioning the population of target nucleic acids into a plurality of subsamples; and/or
(d) prior to the subjecting the population of target nucleic acids to the conversion procedure that affects a first nucleobase of the DNA differently from a second nucleobase of the DNA.

81. The method of any one of the preceding claims, wherein the sequencing comprises sequencing the DNA in a modification-sensitive manner.

82. The method of claim 81, wherein the sequencing in a modification-sensitive manner comprises long-read sequencing.

83. The method of claim 81 or claim 82, wherein the sequencing in a modification-sensitive manner comprises nanopore sequencing.

84. The method of claim 81, wherein the sequencing in a modification-sensitive manner comprises 5-letter or 6-letter sequencing.

85. The method of any one of the preceding claims, wherein the comprises next generation sequencing.

86. The method of any one of the preceding claims, wherein the sequencing comprises generating a plurality of sequencing reads and mapping the plurality of sequencing reads to one or more reference sequences to generate mapped sequence reads.

87. The method of claim 86, further comprising processing mapped sequence reads corresponding to the sequence-variable target region set and to the epigenetic target region set.

88. The method of any one of claims 42-87, wherein the sequencing comprises sequencing at least a portion of the DNA, RNA, or cDNA generated from the RNA of at least the first and second subsamples in the same sequencing cell.

89. The method of any one of the preceding claims, wherein the population of nucleic acids comprises DNA.

90. The method of the immediately preceding claim, wherein the DNA is cell-free DNA.

91. The method of any one of the preceding claims, wherein the population of target nucleic acids is from a blood sample and/or a tissue sample.

92. The method of claim 91, wherein the blood sample is a whole blood sample, a plasma sample, a buffy coat sample, a leukapheresis sample, or a PBMC sample.

93. The method of any one of the preceding claims, wherein the population of target nucleotides is from a subject.

94. The method of claim 93, wherein the subject is an animal.

95. The method of claim 93 or claim 94, wherein the subject is a human.

96. The method of any one of claims 93-95, wherein the subject has or is at risk of having a cancer.

97. The method of any one of claims 93-96, further comprising determining the presence or status of a cancer in the subject.

98. The method of any one of claims 93-97, further comprising determining the likelihood that the subject has an infection.

99. The method of any one of claims 93-98, further comprising determining the likelihood that the subject has a transplant rejection.


SEQUENCE LISTING (14 sequences)

SEQ ID NO: 1 - protein, 397 aa, synthetic construct
MNSNKDKIKVIKVFEAFAGIGSQFKALKNIARSKNWEIQHSGMVEWFVDAIVSYVAIHSKNFNPKIERLDRDILSISNDSKMPISEYGIKKINNTIKASYLNYAKKHFNNLFDIKKVNKDNFPKNIDIFTYSFPCQDLSVQGLQKGIDKELNTRSGLLWEIERILEEIKNSFSKEEMPKYLLMENVKNLLSHKNKKNYNTWLKQLEKFGYKSKTYLLNSKNFDNCQNRERVFCLSIRDDYLEKTGFKFKELEKVKNPPKKIKDILVDSSNYKYLNLNKYETTTFRETKSNIISRPLKNYTTFNSENYVYNINGIGPTLTASGANSRIKIETQQGVRYLTPLECFKYMQFDVNDFKKVQSTNLISENKMIYIAGKSIPVKILEAIFNTLEFVNNEELE

SEQ ID NO: 2 - protein, 397 aa, synthetic construct
MNSNKDKIKVIKVFEAFAGIGSQFKALKNIARSKNWEIQHSGMVEWFVDAIVSYVAIHSKNFNPKIERLDRDILSISNDSKMPISEYGIKKINNTIKASYLNYAKKHFNNLFDIKKVNKDNFPKNIDIFTYSFPCQDLSVQGLQKGIDKELNTRSGLLWEIERILEEIKNSFSKEEMPKYLLMENVKNLLSHKNKKNYNTWLKQLEKFGYKSKTYLLNSKNFDNCQNRERVFCLSIRDDYLEKTGFKFKELEKVKNPPKKIKDILVDSSNYKYLNLNKYETTTFRETKSNIISRPLKNYTTFNSENYVYNINGIGPTLTASGANSRIKIETQQGVRYLTPLECFKYMQFDVNDFKKVQSTNLISENKMIYIAGRSIPVKILEAIFNTLEFVNNEELE

SEQ ID NO: 3 - protein, 507 aa, Homo sapiens
GGSQSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 4 - protein, 871 aa, synthetic construct
QSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCKKKAEPKKAKTKKAARKRSSLENCSSRTEKGKSSSHTKLMENASHMKQMTAQPQLSGPVIRQPPTLQRHLQQGQRPQQPQPPQPQPQTTPQPQPQPQHIMPGNSQSVGSHCSGSTSVYTRQPTPHSPYPSSAHTSDIYGDTNHVNFYPTSSHASGSYLNPSNYMNPYLGLLNQNNQYAPFPYNGSVPVDNGSPFLGSYSPQAQSRDLHRYPNQDHLTNQNLPPIHTLHQQTFGDSPSKYLSYGNQNMQRDAFTTNSTLKPNVHHLATFSPYPTPKMDSHFMGAASRSPYSHPHTDYKTSEHHLPSHTIYSYTAAASGSSSSHAFHNKENDNIANGLSRVLPGFNHDRTASAQELLYSLTGSSQEKQPEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 5 - protein, 474 aa, Homo sapiens
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLVFYQHKSMNEPKHGLALWEAKMAEKAREKEEECEKYG

SEQ ID NO: 6 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLAFYQHKSMNEPKHGLALWEAKMAEKAREKEEECEKYG

SEQ ID NO: 7 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLCFYQHKSMNEPKHGLALWEAKMAEKAREKEEECEKYG

SEQ ID NO: 8 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLGFYQHKSMNEPKHGLALWEAKMAEKAREKEEECEKYG

SEQ ID NO: 9 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLIFYQHKSMNEPKHGLALWEAKMAEKAREKEEECEKYG

SEQ ID NO: 10 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLPFYQHKSMNEPKHGLALWEAKMAEKAREKEEECEKYG

SEQ ID NO: 11 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVSACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLVFYQHKSMNEPKHGLALWEAKMAEKAREKEEECEKYG

SEQ ID NO: 12 - protein, 878 aa, synthetic construct
MGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVSACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 13 - protein, 9 aa, synthetic construct
FSGVTACLD

SEQ ID NO: 14 - protein, 9 aa, synthetic construct
FSGVSACLD